SEQUENCE LISTING (49 sequences)

SEQ ID NO: 1 - protein, 75 aa, Artificial sequence
  Synthetic
MQIFVKTLTGKTITLEVEPSDTIENVKAKIQDKEGIPPDQQRLIFAGKQLEDGRTLSDYNIQKESTLHLVLRLRG

SEQ ID NO: 2 - protein, 74 aa, Artificial sequence
  Synthetic
MLIFVKTLTGKTITLEVEPSDTIENVKAKIQDKEGIPPDQQRLAFAGKSLEDGRTLSDYNILKDSKLHPLLRLR

SEQ ID NO: 3 - DNA, 100 bp, Artificial sequence
  Synthetic
tgttatattatgcacagccaaactctctgcggtctgcccacgtacaatgcgtctgggtgaaaaatgtaagtccagatcatttagtaataggaaattaaat

SEQ ID NO: 4 - protein, 74 aa, Artificial sequence; identical to SEQ ID NO: 2
  Synthetic

SEQ ID NO: 5 - RNA, 225 nt, Artificial sequence
  Synthetic
  modified_base (1)..(225): U=Pseudouridine
auguugaucuuuguaaaaacucugacgggaaaaacgauaacuuuggaaguggagccguccgacacuauagagaauguaaaagcuaaaauucaggacaaagaaggcauaccaccugaccaacagcggcuggccuuugcgggaaagagccuugaggacggcaggacucuuucagauuacaacauccucaaggacuccaaacugcauccucuucugcgccuuagguga

SEQ ID NO: 6 - RNA, 473 nt, Artificial sequence
  Synthetic
  misc_feature (1)..(473): U=Pseudouridine
ggguaauacaagcuugcuuguucuuuuugcagaagcucagaauaaacgcucaacuuuggcagccaccauguugaucuuuguaaaaacucugacgggaaaaacgauaacuuuggaaguggagccguccgacacuauagagaauguaaaagcuaaaauucaggacaaagaaggcauaccaccugaccaacagcggcuggccuuugcgggaaagagccuugaggacggcaggacucuuucagauuacaacauccucaaggacuccaaacugcauccucuucugcgccuuaggugaugauaucacuagugacugacuaggaucugguuaccacuaaaccagccucaagaacacccgaauggagucucuaagcuacauaauaccaacuuacacuuuacaaaauguugucccccaaaauguagccauucguaucugcuccuaauaaaaagaaaguuucuucacauucugcuagcucuag

SEQ ID NO: 7 - protein, 96 aa, Artificial sequence
  Synthetic
MDCPGRDRRTEEENLRKKGEPHHELPPGSTKRALPNNTSSSPQPKKKPLDGEYFTLQIRGRERFEMFRELNEALELKDAQAGKEPGGSRAHSSHLK

SEQ ID NO: 8 - RNA, 291 nt, Artificial sequence
  Synthetic
  misc_feature (1)..(291): U=Pseudouridine
auggauuguccugggagagaccggcgcacagaggaagagaaucuccgcaagaaaggggagccucaccacgagcugcccccagggagcacuaagcgagcacugcccaacaacaccagcuccucuccccagccaaagaagaaaccacuggauggagaauauuucacccuucagauccgugggcgugagcgcuucgagauguuccgagagcugaaugaggccuuggaacucaaggaugcccaggcugggaaggagccaggggggagcagggcucacuccagccaccugaaguag

SEQ ID NO: 9 - RNA, 539 nt, Artificial sequence
  Synthetic
  misc_feature (1)..(539): U=Pseudouridine
ggguaauacaagcuugcuuguucuuuuugcagaagcucagaauaaacgcucaacuuuggcagccaccauggauuguccugggagagaccggcgcacagaggaagagaaucuccgcaagaaaggggagccucaccacgagcugcccccagggagcacuaagcgagcacugcccaacaacaccagcuccucuccccagccaaagaagaaaccacuggauggagaauauuucacccuucagauccgugggcgugagcgcuucgagauguuccgagagcugaaugaggccuuggaacucaaggaugcccaggcugggaaggagccaggggggagcagggcucacuccagccaccugaaguagugauaucacuagugacugacuaggaucugguuaccacuaaaccagccucaagaacacccgaauggagucucuaagcuacauaauaccaacuuacacuuuacaaaauguugucccccaaaauguagccauucguaucugcuccuaauaaaaagaaaguuucuucacauucugcuagcucuag

SEQ ID NO: 10 - protein, 1389 aa, Artificial sequence
  Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGPPKKKRKVYPYDVPDYA

SEQ ID NO: 11 - RNA, 4170 nt, Artificial sequence
  Synthetic
  misc_feature (1)..(4170): U=Pseudouridine
auggacaagaaguacagcaucggccuggacaucgguaccaacagcgugggcugggccgugaucaccgacgaguacaaggugcccagcaagaaguucaaggugcugggcaacaccgaccgccacagcaucaagaagaaccugaucggcgcccugcuguucgacagcggcgagaccgccgaggccacccgccugaagcgcaccgcccgccgccgcuacacccgccgcaagaaccgcaucugcuaccugcaggagaucuucagcaacgagauggccaagguggacgacagcuucuuccaccgccuggaggagagcuuccugguggaggaggacaagaagcacgagcgccaccccaucuucggcaacaucguggacgagguggccuaccacgagaaguaccccaccaucuaccaccugcgcaagaagcugguggacagcaccgacaaggccgaccugcgccugaucuaccuggcccuggcccacaugaucaaguuccgcggccacuuccugaucgagggcgaccugaaccccgacaacagcgacguggacaagcuguucauccagcuggugcagaccuacaaccagcuguucgaggagaaccccaucaacgccagcggcguggacgccaaggccauccugagcgcccgccugagcaagagccgccgccuggagaaccugaucgcccagcugcccggcgagaagaagaacggccuguucggcaaccugaucgcccugagccugggccugacccccaacuucaagagcaacuucgaccuggccgaggacgccaagcugcagcugagcaaggacaccuacgacgacgaccuggacaaccugcuggcccagaucggcgaccaguacgccgaccuguuccuggccgccaagaaccugagcgacgccauccugcugagcgacauccugcgcgugaacaccgagaucaccaaggccccccugagcgccagcaugaucaagcgcuacgacgagcaccaccaggaccugacccugcugaaggcccuggugcgccagcagcugcccgagaaguacaaggagaucuucuucgaccagagcaagaacggcuacgccggcuacaucgacggcggcgccagccaggaggaguucuacaaguucaucaagcccauccuggagaagauggacggcaccgaggagcugcuggugaagcugaaccgcgaggaccugcugcgcaagcagcgcaccuucgacaacggcagcaucccccaccagauccaccugggcgagcugcacgccauccugcgccgccaggaggacuucuaccccuuccugaaggacaaccgcgagaagaucgagaagauccugaccuuccgcauccccuacuacgugggcccccuggcccgcggcaacagccgcuucgccuggaugacccgcaagagcgaggagaccaucacccccuggaacuucgaggaggugguggacaagggcgccagcgcccagagcuucaucgagcgcaugaccaacuucgacaagaaccugcccaacgagaaggugcugcccaagcacagccugcuguacgaguacuucaccguguacaacgagcugaccaaggugaaguacgugaccgagggcaugcgcaagcccgccuuccugagcggcgagcagaagaaggccaucguggaccugcuguucaagaccaaccgcaaggugaccgugaagcagcugaaggaggacuacuucaagaagaucgagugcuucgacagcguggagaucagcggcguggaggaccgcuucaacgccagccugggcaccuaccacgaccugcugaagaucaucaaggacaaggacuuccuggacaacgaggagaacgaggacauccuggaggacaucgugcugacccugacccuguucgaggaccgcgagaugaucgaggagcgccugaagaccuacgcccaccuguucgacgacaaggugaugaagcagcugaagcgccgccgcuacaccggcuggggccgccugagccgcaagcuuaucaacggcauccgcgacaagcagagcggcaagaccauccuggacuuccugaagagcgacggcuucgccaaccgcaacuucaugcagcugauccacgacgacagccugaccuucaaggaggacauccagaaggcccaggugagcggccagggcgacagccugcacgagcacaucgccaaccuggccggcagccccgccaucaagaagggcauccugcagaccgugaaggugguggacgagcuggugaaggugaugggccgccacaagcccgagaacaucgugaucgagauggcccgcgagaaccagaccacccagaagggccagaagaacagccgcgagcgcaugaagcgcaucgaggagggcaucaaggagcugggcagccagauccugaaggagcaccccguggagaacacccagcugcagaacgagaagcuguaccuguacuaccugcagaacggccgcgacauguacguggaccaggagcuggacaucaaccgccugagcgacuacgacguggaccacaucgugccccagagcuuccugaaggacgacagcaucgacaacaaggugcugacccgcagcgacaagaaccgcggcaagagcgacaacgugcccagcgaggagguggugaagaagaugaagaacuacuggcgccagcugcugaacgccaagcugaucacccagcgcaaguucgacaaccugaccaaggccgagcgcggcggccugagcgagcuggacaaggccggcuucaucaagcgccagcugguggagacccgccagaucaccaagcacguggcccagauccuggacagccgcaugaacaccaaguacgacgagaacgacaagcugauccgcgaggugaaggugaucacccugaagagcaagcuggugagcgacuuccgcaaggacuuccaguucuacaaggugcgcgagaucaacaacuaccaccacgcccacgacgccuaccugaacgccguggugggcaccgcccugaucaagaaguaccccaagcuggagagcgaguucguguacggcgacuacaagguguacgacgugcgcaagaugaucgccaagagcgagcaggagaucggcaaggccaccgccaaguacuucuucuacagcaacaucaugaacuucuucaagaccgagaucacccuggccaacggcgagauccgcaagcgcccccugaucgagaccaacggcgagaccggcgagaucgugugggacaagggccgcgacuucgccaccgugcgcaaggugcugagcaugccccaggugaacaucgugaagaagaccgaggugcagaccggcggcuucagcaaggagagcauccugcccaagcgcaacagcgacaagcugaucgcccgcaagaaggacugggaccccaagaaguacggcggcuucgacagccccaccguggccuacagcgugcuggugguggccaagguggagaagggcaagagcaagaagcugaagagcgugaaggagcugcugggcaucaccaucauggagcgcagcagcuucgagaagaaccccaucgacuuccuggaggccaagggcuacaaggaggugaagaaggaccugaucaucaagcugcccaaguacagccuguucgagcuggagaacggccgcaagcgcauguuagccagcgccggcgaguuacagaaggggaaugagcuagcuuuacccagcaaguacgugaacuuccuguaccuggccagccacuacgagaagcugaagggcagccccgaggacaacgagcagaagcagcuguucguggagcagcacaagcacuaccuggacgagaucaucgagcagaucagcgaguucagcaagcgcgugauccuggccgacgccaaccuggacaaggugcugagcgccuacaacaagcaccgcgacaagcccauccgcgagcaggccgagaacaucauccaccuguucacccugaccaaccugggcgcccccgccgccuucaaguacuucgacaccaccaucgaccgcaagcgcuacaccagcaccaaggaggugcuggacgccacccugauccaccagagcaucaccggucuguacgagacccgcaucgaccugagccagcugggcggcgacggcggcuccggaccuccaaagaaaaagagaaaaguauaccccuacgacgugcccgacuacgccuaa

SEQ ID NO: 12 - RNA, 4211 nt, Artificial sequence
  Synthetic
  misc_feature (1)..(4211): U=Pseudouridine
ggaauacaaaaguuugccaccauggacaagaaguacagcaucggccuggacaucgguaccaacagcgugggcugggccgugaucaccgacgaguacaaggugcccagcaagaaguucaaggugcugggcaacaccgaccgccacagcaucaagaagaaccugaucggcgcccugcuguucgacagcggcgagaccgccgaggccacccgccugaagcgcaccgcccgccgccgcuacacccgccgcaagaaccgcaucugcuaccugcaggagaucuucagcaacgagauggccaagguggacgacagcuucuuccaccgccuggaggagagcuuccugguggaggaggacaagaagcacgagcgccaccccaucuucggcaacaucguggacgagguggccuaccacgagaaguaccccaccaucuaccaccugcgcaagaagcugguggacagcaccgacaaggccgaccugcgccugaucuaccuggcccuggcccacaugaucaaguuccgcggccacuuccugaucgagggcgaccugaaccccgacaacagcgacguggacaagcuguucauccagcuggugcagaccuacaaccagcuguucgaggagaaccccaucaacgccagcggcguggacgccaaggccauccugagcgcccgccugagcaagagccgccgccuggagaaccugaucgcccagcugcccggcgagaagaagaacggccuguucggcaaccugaucgcccugagccugggccugacccccaacuucaagagcaacuucgaccuggccgaggacgccaagcugcagcugagcaaggacaccuacgacgacgaccuggacaaccugcuggcccagaucggcgaccaguacgccgaccuguuccuggccgccaagaaccugagcgacgccauccugcugagcgacauccugcgcgugaacaccgagaucaccaaggccccccugagcgccagcaugaucaagcgcuacgacgagcaccaccaggaccugacccugcugaaggcccuggugcgccagcagcugcccgagaaguacaaggagaucuucuucgaccagagcaagaacggcuacgccggcuacaucgacggcggcgccagccaggaggaguucuacaaguucaucaagcccauccuggagaagauggacggcaccgaggagcugcuggugaagcugaaccgcgaggaccugcugcgcaagcagcgcaccuucgacaacggcagcaucccccaccagauccaccugggcgagcugcacgccauccugcgccgccaggaggacuucuaccccuuccugaaggacaaccgcgagaagaucgagaagauccugaccuuccgcauccccuacuacgugggcccccuggcccgcggcaacagccgcuucgccuggaugacccgcaagagcgaggagaccaucacccccuggaacuucgaggaggugguggacaagggcgccagcgcccagagcuucaucgagcgcaugaccaacuucgacaagaaccugcccaacgagaaggugcugcccaagcacagccugcuguacgaguacuucaccguguacaacgagcugaccaaggugaaguacgugaccgagggcaugcgcaagcccgccuuccugagcggcgagcagaagaaggccaucguggaccugcuguucaagaccaaccgcaaggugaccgugaagcagcugaaggaggacuacuucaagaagaucgagugcuucgacagcguggagaucagcggcguggaggaccgcuucaacgccagccugggcaccuaccacgaccugcugaagaucaucaaggacaaggacuuccuggacaacgaggagaacgaggacauccuggaggacaucgugcugacccugacccuguucgaggaccgcgagaugaucgaggagcgccugaagaccuacgcccaccuguucgacgacaaggugaugaagcagcugaagcgccgccgcuacaccggcuggggccgccugagccgcaagcuuaucaacggcauccgcgacaagcagagcggcaagaccauccuggacuuccugaagagcgacggcuucgccaaccgcaacuucaugcagcugauccacgacgacagccugaccuucaaggaggacauccagaaggcccaggugagcggccagggcgacagccugcacgagcacaucgccaaccuggccggcagccccgccaucaagaagggcauccugcagaccgugaaggugguggacgagcuggugaaggugaugggccgccacaagcccgagaacaucgugaucgagauggcccgcgagaaccagaccacccagaagggccagaagaacagccgcgagcgcaugaagcgcaucgaggagggcaucaaggagcugggcagccagauccugaaggagcaccccguggagaacacccagcugcagaacgagaagcuguaccuguacuaccugcagaacggccgcgacauguacguggaccaggagcuggacaucaaccgccugagcgacuacgacguggaccacaucgugccccagagcuuccugaaggacgacagcaucgacaacaaggugcugacccgcagcgacaagaaccgcggcaagagcgacaacgugcccagcgaggagguggugaagaagaugaagaacuacuggcgccagcugcugaacgccaagcugaucacccagcgcaaguucgacaaccugaccaaggccgagcgcggcggccugagcgagcuggacaaggccggcuucaucaagcgccagcugguggagacccgccagaucaccaagcacguggcccagauccuggacagccgcaugaacaccaaguacgacgagaacgacaagcugauccgcgaggugaaggugaucacccugaagagcaagcuggugagcgacuuccgcaaggacuuccaguucuacaaggugcgcgagaucaacaacuaccaccacgcccacgacgccuaccugaacgccguggugggcaccgcccugaucaagaaguaccccaagcuggagagcgaguucguguacggcgacuacaagguguacgacgugcgcaagaugaucgccaagagcgagcaggagaucggcaaggccaccgccaaguacuucuucuacagcaacaucaugaacuucuucaagaccgagaucacccuggccaacggcgagauccgcaagcgcccccugaucgagaccaacggcgagaccggcgagaucgugugggacaagggccgcgacuucgccaccgugcgcaaggugcugagcaugccccaggugaacaucgugaagaagaccgaggugcagaccggcggcuucagcaaggagagcauccugcccaagcgcaacagcgacaagcugaucgcccgcaagaaggacugggaccccaagaaguacggcggcuucgacagccccaccguggccuacagcgugcuggugguggccaagguggagaagggcaagagcaagaagcugaagagcgugaaggagcugcugggcaucaccaucauggagcgcagcagcuucgagaagaaccccaucgacuuccuggaggccaagggcuacaaggaggugaagaaggaccugaucaucaagcugcccaaguacagccuguucgagcuggagaacggccgcaagcgcauguuagccagcgccggcgaguuacagaaggggaaugagcuagcuuuacccagcaaguacgugaacuuccuguaccuggccagccacuacgagaagcugaagggcagccccgaggacaacgagcagaagcagcuguucguggagcagcacaagcacuaccuggacgagaucaucgagcagaucagcgaguucagcaagcgcgugauccuggccgacgccaaccuggacaaggugcugagcgccuacaacaagcaccgcgacaagcccauccgcgagcaggccgagaacaucauccaccuguucacccugaccaaccugggcgcccccgccgccuucaaguacuucgacaccaccaucgaccgcaagcgcuacaccagcaccaaggaggugcuggacgccacccugauccaccagagcaucaccggucuguacgagacccgcaucgaccugagccagcugggcggcgacggcggcuccggaccuccaaagaaaaagagaaaaguauaccccuacgacgugcccgacuacgccuaauaacucgagcaugcaucuag

SEQ ID NO: 13 - DNA, 5996 bp, Artificial sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaagcttccatgggcgcggccgcaatgagtctttgaaagattctgttctagcaacttcaagccattttggactccgaaaactccgcaatatttcacaactgcgcatgcgtgcttggaaagcgtcagcgcgctagatgtttacgtatcgagaaaggggcgggtcattatctttagcggaccaatgaaaacgctccagattatcttttttctattggccgaccggggccaattagaaatcaaccttcttttagcgggcgtgtagcgccagcgcgctgtgacgtaatgtgaggggtctcccggcagggctgagctggaccaatgaggaaaggcaaggggccgatttgcctgttctcacgccccaccctcagacctagccggagcaaagtttcacttatagaagggagaggagcgaacatggcagcgatgcggaaaggcaaaggccccatatgcctcttttcacgacccacgctgcgaccgtcccgatcaaaagtatccttgattgagggccggggcgctaatatggccgctaggtggagattctggtgcgtctccgtcactatggttgtagccttgcttattgtctgcgatgtgccgtccgcttctgcgcaacggaagaaagaaatggttctttctgaaaaagtatcacaactcatggagtggacaaataagagacccgtgatacgaatgaacggagataagtttcggcggctcgtaaaggcacccccacgaaactattcagtgatagtcatgtttactgcacttcagttgcatcgacagtgcgtggtttgcaaacaggcggatgaagagttccagattctcgcaaactcatggaggtactccagcgccttcaccaatcgaatcttctttgcgatggtcgacttcgacgagggatccgatgtttttcagatgctcaacatgaactcagctcctacgttcattaactttccggctaaaggcaaaccgaagagaggagacacttacgagctgcaggtccgggggttttccgcagaacagatagcacgatggatagcagaccgaaccgacgtgaatatccgggtgatccgcccacccaattacgcaggtccattgatgctgggattgctcttggctgtgatcggcgggcttgtttatttgcggaggtcaaatatggaattccttttcaataagacgggatgggcgttcgcagccttgtgtttcgttctcgcaatgacgtccgggcagatgtggaatcatatacggggtccgccgtacgcgcacaagaatccccacacgggtcacgtcaattatattcatggcagctcacaagcccagttcgtcgctgagacgcacattgtacttctcttcaacggtggggtaaccctcgggatggtccttctgtgtgaggcagcaactagcgatatggatataggtaaaagaaagatcatgtgcgtagcaggtatcggtctcgtcgtgctttttttttcatggatgttgtctatctttcgatcaaagtatcacggttacccatactctttcttgatgtcctaaaatcagccgctggactataaaatctgtgaaagacttactgggatactgaactacgttgcgccgttcactctgtgtgggtacgcagccttgatgccactttaccatgctatagcatctcgcatggccttcatcttcagtagtttgtacaagagctggctgttgagcctctatgaagaactctggcccgtagtgcggcaacgaggcgtagtctgcactgtatttgctgatgcgacaccaactgggtggggaatagctactacttgccagctccttagtggcaccttcgctttccctcttccgatagcgactgcggagctcattgccgcctgcctcgccaggtgctggacgggtgcccggcttctggggactgataatagcgttgttcttagtggaaagttgacgtcatttccctggttgctggcctgcgtggccacgtggatactgaggggcactagcttttgctatgtccccagtgctctgaatccggctgaccttccctcaaggggacttcttcctgcactccgcccactcccacgacttcggttgaggccacagacatctcggatctctctttgggcggcttcccctcaccgattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatctctagacgttggcggttttggtgtgtctctgtgaccatggtggtggcgctgctcatcgtttgcgacgttccctcagcctctgcccaaagaaagaaggaggtgagaacgcggtttccagcagcatgggcttttcccaatgactggggcttaagagggtctgttcgcctcttcccagccccctttccctgccgcttctatgcctttggtggcttcaattacgcggttttcggagttgtggaattcgtgagagaaggaactgtaactctttgtcttctggggatgtagaatctgagagtggagtagcgtaggtgacagcccgacttctacagtcttgtgaatgtgcatagctatgagttttgacacttttgagtgtgggccccgtcaggtgttgggggtggggagttcgctcgagtcgagcgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 19 bp, Artificial sequence
  Synthetic
gagcgaacatggcgcgcgt

SEQ ID NO: 15 - DNA, 20 bp, Artificial sequence
  Synthetic
agcctctgcccaaagaaaga

SEQ ID NO: 16 - DNA, 20 bp, Artificial sequence
  Synthetic
agtgaaactttgctccggct

SEQ ID NO: 17 - DNA, 19 bp, Artificial sequence
  Synthetic
cttgctccggctaggtctg

SEQ ID NO: 18 - DNA, 20 bp, Artificial sequence
  Synthetic
gtgtgtctctgtgaccatgg

SEQ ID NO: 19 - DNA, 20 bp, Artificial sequence
  Synthetic
gatgagcagcgccaccacca

SEQ ID NO: 20 - DNA, 20 bp, Artificial sequence
  Synthetic
tggaccaatgaggaaaggca

SEQ ID NO: 21 - DNA, 20 bp, Artificial sequence
  Synthetic
gaccaatgaggaaaggcaag

SEQ ID NO: 22 - DNA, 20 bp, Artificial sequence
  Synthetic
tttgctccggctaggtctga

SEQ ID NO: 23 - DNA, 20 bp, Artificial sequence
  Synthetic
gccccaccctcagacctagc

SEQ ID NO: 24 - DNA, 17 bp, Artificial sequence
  Synthetic
cgaaggcctctgctagg

SEQ ID NO: 25 - DNA, 17 bp, Artificial sequence
  Synthetic
gggcagctaagaagcca

SEQ ID NO: 26 - DNA, 21 bp, Artificial sequence
  Synthetic
aaaggcagtgagttgaagttt

SEQ ID NO: 27 - DNA, 25 bp, Artificial sequence
  Synthetic
gagtatgtaagaacagcaggataaa

SEQ ID NO: 28 - DNA, 17 bp, Artificial sequence
  Synthetic
ctgtctcacccgcactg

SEQ ID NO: 29 - DNA, 17 bp, Artificial sequence
  Synthetic
aaagccaccaccctgac

SEQ ID NO: 30 - DNA, 22 bp, Artificial sequence
  Synthetic
gatcttgaggtgtccctgattc

SEQ ID NO: 31 - DNA, 22 bp, Artificial sequence
  Synthetic
ctctgaagtgctctctgttgag

SEQ ID NO: 32 - DNA, 18 bp, Artificial sequence
  Synthetic
agcagggtgcctacttct

SEQ ID NO: 33 - DNA, 20 bp, Artificial sequence
  Synthetic
tcctcctcacactcgttctt

SEQ ID NO: 34 - DNA, 23 bp, Artificial sequence
  Synthetic
agaaggttgaagtagtgacttgt

SEQ ID NO: 35 - DNA, 25 bp, Artificial sequence
  Synthetic
ggaaacttaacatctgatcagcaaa

SEQ ID NO: 36 - DNA, 21 bp, Artificial sequence
  Synthetic
atggctgtgataacaagcaga

SEQ ID NO: 37 - DNA, 26 bp, Artificial sequence
  Synthetic
cttcagggatattcttcagtagactt

SEQ ID NO: 38 - DNA, 21 bp, Artificial sequence
  Synthetic
cttgattcttgattggctctg

SEQ ID NO: 39 - DNA, 19 bp, Artificial sequence
  Synthetic
gccagcccatgtttagaag

SEQ ID NO: 40 - DNA, 20 bp, Artificial sequence
  Synthetic
gaccaatgaaaacgctccag

SEQ ID NO: 41 - DNA, 21 bp, Artificial sequence
  Synthetic
atcggccagcagtatagagtc

SEQ ID NO: 42 - DNA, 22 bp, Artificial sequence
  Synthetic
gaggcaggataacggactaaat

SEQ ID NO: 43 - DNA, 20 bp, Artificial sequence
  Synthetic
tccacctagcggccatatta

SEQ ID NO: 44 - DNA, 25 bp, Artificial sequence
  Synthetic
aaagtatccttgattgagggccggg

SEQ ID NO: 45 - DNA, 300 bp, Artificial sequence
  Synthetic
atggaggagccgcagtcagatcctagcgtcaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 46 - protein, 393 aa, Artificial sequence
  Synthetic
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPSGTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 47 - protein, 97 aa, Artificial sequence
  Synthetic
MEEPQSDPSVKRALPNNTSSSPQPKKKPLDEYFTLQIRGRERFEMFRELNEALELKDAQAGKEPGGSRAHSSHLKSKKGQSTRHKKLMFKTEGPDSD

SEQ ID NO: 48 - protein, 97 aa, Artificial sequence
  Synthetic
MDCPGRDRRTEEENLRKKGEPHHELPPGSTKRALPNNTSSSPQPKKKPLDGEYFTLQIRGRERFEMFRELNEALLELKDAQAGKEPGGSRAHSSHLK

SEQ ID NO: 49 - DNA, 23 bp, Artificial sequence
  Synthetic
  misc_feature (21)..(21): n is a, c, g, or t
gagcgaagatggcagcgcgtngg

(19) United States
(12) Patent Application Publication
(10) Pub. No.: US 2024/0181005 A1
(43) Pub. Date: Jun. 6, 2024

(54) COMPOSITIONS, SYSTEMS, AND METHODS FOR GENERATING GENE-EDITED CELLS

(71) Applicants: CellScript, LLC, Madison, WI (US); THE UNITED STATES OF AMERICA, AS REPRESENTED BY THE SECRETARY, DEPARTMENT OF HEALTH AND HUMAN SERVIC, Bethesda, MD (US)

(72) Inventors: Ronald Meis, Fitchburg, WI (US); Gary Dahl, Madison, WI (US); Suk See De Ravin, Bethesda, MD (US); Julie Brault, Bethesda, MD (US); Colin L. Sweeney, Bethesda, MD (US); Harry L. Malech, Bethesda, MD (US)

(21) Appl. No.: 17/998,752

(22) PCT Filed: May 17, 2021
(86) PCT No.: PCT/US2021/032782

§ 371 Date: Nov. 14, 2022

Related U.S. Application Data
(60) Provisional application No. 63/025,815, filed on May 15, 2020.

(51) Int. Cl. A61K 38/17; A61K 31/4184; A61K 31/428; A61K 31/44; A61K 31/7105; A61K 35/17; A61K 35/28; A61K 35/545; A61K 38/46; A61K 48/00; A61P 37/04; C12N 9/22; C12N 15/11; C12N 15/86; C12N 15/90

(52) U.S. Cl. CPC A61K 38/177; A61K 31/4184; A61K 31/428; A61K 31/44; A61K 31/7105; A61K 35/17; A61K 35/28; A61K 35/545; A61K 38/465; A61K 48/0033; A61P 37/04; C12N 9/22; C12N 15/11; C12N 15/86; C12N 15/907; C12N 2310/20; C12N 2750/14143; C12N 2800/80

(57) ABSTRACT

The present invention relates to compositions, systems, and methods for editing a disease/condition causing mutation region in a target gene in a cell. In certain embodiments, the following components are employed: i) mRNA encoding a Tumor Protein p53 (TP53) inhibitor, ii) an inhibiting agent that inhibits Tumor Suppressor p53-Binding Protein 1 (53BPI) (e.g., small molecule EoHR or mRNA encoding a protein that inhibits 53BPI), iii) mRNA encoding a Cas nuclease for CRISPR; iv) a guide RNA specific for a target cleavage site proximal to said disease/condition-causing mutation region; and v) a repair template comprising a region of interest configured to replace said disease/condition-causing mutation region in the target gene during homology-directed repair (HDR). In certain embodiments, the cell is a T-cell, stem cell (e.g., hematopoietic stem cell), or progenitor cell from a subject with the disease or condition (e.g., a Primary Immunodeficiency Disease (PID)). In some embodiments, the gene-edited cell is administered to the subject.

The present application claims priority to U.S. Provisional application Ser. No. 63/025,815 filed May 15, 2020, which is herein incorporated by reference in its entirety.

This invention was made with government support under Z01-AI-000988 and 000644, awarded by the National Institutes of Health. The government has certain rights in the invention.

### FIELD OF THE INVENTION

The present invention relates to compositions, systems, and methods for editing a disease/condition-causing mutation region in a target gene in a cell. In certain embodiments, the following components are employed: i) mRNA encoding a Tumor Protein p53 (TP53) inhibitor, ii) an inhibiting agent that inhibits Tumor Suppressor p53-Binding Protein 1 (53BPI) (e.g., small molecule EoHR or mRNA encoding a protein that inhibits 53BPI), iii) mRNA encoding a Cas nuclease for CRISPR; iv) a guide RNA specific for a target cleavage site proximal to said disease/condition-causing mutation region; and v) a repair template comprising a region of interest configured to replace said disease/condition-causing mutation region in the target gene during homology-directed repair (HDR). In certain embodiments, the cell is a T-cell, stem cell (e.g., hematopoietic stem cell), or hematopoietic progenitor cell from a subject with the disease or condition (e.g., a Primary Immunodeficiency Disease (PID)). In some embodiments, the gene-edited cell is administered to the subject (e.g., into bone marrow).

### BACKGROUND

While most people have intact immune systems that serve to protect them from the wide variety of infectious organisms that commonly infect people including viruses, bacteria and fungi, many individuals have impaired or compromised immunity. There are many components to the immune system all of which cooperate to reject foreign invading pathogens. The humoral immune system that produces circulating antibody is one of the principal components that is often found to be lacking in immunocompromised individuals either at birth or may be a defect that is acquired. Immunodeficiency may be classified as primary or secondary.

Primary Immunodeficiency Diseases (PIDs) are a group of more than 150 diseases in which part of a subject's immune system is missing or does not function normally. To be considered a primary immunodeficiency, the cause of the immune deficiency must not be secondary in nature (e.g., caused by other disease, drug treatment, or environmental exposure to toxins). Most primary immunodeficiencies are genetic disorders and are diagnosed in children, although less severe forms may not be recognized until adulthood. About 1 in 500 people are born with a primary immunodeficiency.

Most immunodeficiencies (e.g., primary and secondary) result in a faulty humoral or cell mediated immune response toward infectious pathogens. The absence of a healthy, properly functioning humoral immune system (that part of the immune system required for generation of antibodies that are ultimately responsible for eradicating infection) renders a person susceptible to many infections. Infusion of immunoglobulin has been shown to reconstitute the ability of these immune defective individuals to defend themselves against infection.

Primary Immunodeficiency Diseases (PIDs) due to loss-of-function gene mutations frequently increase risk of infections due to dysfunctional immune cells. Chronic granulomatous disease (CGD) is an example of an inherited genetic disorder caused by mutations in genes encoding the subunits of the phagocyte NADPH oxidase complex, including gp91phox, p47phox, p67phox, p22phox and p40phox In healthy subjects, the phagocyte NADPH oxidase produces superoxide anion that is subsequently transformed into other reactive oxidative species (ROS) critical for host defenses. Despite significant improvements in antimicrobial prophylaxis, patients with CGD remain at high risk for invasive infections by a pathognomonic group of microbes including Staphylococcus, Burkholderia, Nocardia, Serratia, Klebsiella, Aspergillus, as well as newly emerging microorganisms. Serious infections occur at rates of 0.3-0.4/year and remain the primary cause of morbidity and early mortality in CGD. Hematopoietic stem cell transplants (HSCT) from allogeneic donors provide potential for definitive cures and have also been used for treatment of severe infections that have exhausted medical options (Parta et al., J Clin Immunol 35:675-680, 2015).

Magnesium transporter 1 (MAGT1) maintains magnesium homeostasis and its deficiency impairs N-linked glycosylation of key immune proteins (1-3). This causes the primary immunodeficiency (PID) ‘X-linked MAGT1 deficiency with increased susceptibility to EBV-infection and N-linked glycosylation defect’ (XMEN) disease (3-6) presenting with a broad spectrum of clinical manifestations including susceptibility to infections, autoimmunity and lymphoproliferative disease (LPD), and more recently, extra-immune manifestations as neurological complications. A well characterized defect in MAGT1 deficiency is the abrogation of NKG2D receptor expression specifically on CD8⁺ T and NK cells, which causes susceptibility to EBV and LPD (7-10). Magnesium supplements have not been effective, and hematopoietic stem cell (HSCs) transplant has been associated with high mortality rates (4, 11-13).

### SUMMARY OF THE INVENTION

The present invention relates to compositions, systems, and methods for editing a disease/condition-causing mutation region in a target gene in a cell. In certain embodiments, the following components are employed: i) mRNA encoding a Tumor Protein p53 (TP53) inhibitor, ii) an inhibiting agent that inhibits Tumor Suppressor p53-Binding Protein 1 (53BPI) (e.g., small molecule EoHR or mRNA encoding a protein that inhibits 53BPI), iii) mRNA encoding a CRISPR-Cas nuclease; iv) a guide RNA specific for a target cleavage site proximal to said disease/condition-causing mutation region; and v) a repair template comprising a region of interest configured to replace said disease/condition-causing mutation region in the target gene during homology-directed repair (HDR).

In some embodiments, provided herein are composition and systems comprising: a) a human or animal cell having a mutation in its genomic DNA that is responsible for a disease or condition in a subject from which the cell was derived; b) a first mRNA encoding a first protein, wherein the first protein is capable of reducing, suppressing or preventing an innate immune response, and c) a second mRNA encoding a second protein that inhibits 53BPI, d) a third mRNA encoding a third protein comprising a Cas endonuclease for use in CRISPR DNA editing by homology directed repair, and e) guide RNA comprising a tracrRNA having a sequence that binds to the Cas nuclease and a CRISPR RNA (crRNA) having a short “guide” sequence that is complementary to and binds to a specific target cleavage site in the genomic DNA that is near to the mutation.

In particular embodiments, provided herein are compositions comprising: a)mRNA encoding a first protein, wherein the first protein is capable of reducing, suppressing or preventing an innate immune response in a human or animal cell, and wherein a mutation in the genome of the cell is responsible for a disease or condition in a human or animal subject; b) a second mRNA encoding a second protein that inhibits 53BPI, c) mRNA encoding a third protein, wherein the third protein comprises a Cas nuclease for CRISPR; and d) guide RNA (gRNA or sgRNA) comprising a tracrRNA having a sequence that binds to a Cas enzyme and a CRISPR RNA (crRNA) having a short “guide” sequence that is complementary to and binds to a specific target cleavage site in the genome that is near to the mutation.

In particular embodiments, provided herein are use of any of the compositions herein for preparing a pharmaceutical composition for treating a condition described herein. In some embodiments, provided herein are use of any of the compositions herein for preparing CRISPR/Cas gene-edited cells for treating a condition herein. In further embodiments, provided herein are use of the CRISPR/Cas gene-edited cells herein for the treatment of a condition herein. In further embodiments, provided herein are use of any of compositions herein preparing mutation-corrected cells for treating a condition herein. In some embodiments, provided herein use of the mutation-corrected cells herein for preparing a pharmaceutical composition for treatment of a condition herein. In other embodiments, provided herein is use of the mutation-corrected cells herein in the preparation of a pharmaceutical composition for treatment of a mutation-causing condition herein.

In certain embodiments, provided herein are compositions or systems comprising: a) eukaryotic cells having a mutation in its genomic DNA that is responsible for a disease or condition in a the eukaryotic subject from which the cells were derived, and b) a first mRNA encoding a first protein, wherein the first protein is capable of reducing, suppressing or preventing an innate immune response comprising secretion of pro-inflammatory or inflammatory cytokines by the human or animal cell in response to a stress event such as double-stranded breaks in its genomic DNA, and c) a second mRNA encoding a second protein that is capable of binding to the portion of the Tudor domain of p53-Binding Protein 1 (53BP1) that also is capable of binding to the dimethylated lysine20 on the N-terminal tail of histone 4 (H4K20me2) to initiate a double-stranded-break (DSB) response that ultimately leads to the DNA repair by non-homologous end joining (NHEJ) by DNA ligase IV, wherein the binding of the second protein to the 53BP1 blocks TP53 binding to H4K20me2 of histone 4, inhibits or reduces DNA repair by NHEJ and increase repair by homology directed repair (HDR), which is less error prone, by about 2-fold, and d) a third mRNA encoding a third protein comprising an endonuclease for use in CRISPR/Cas DNA editing by homology directed repair; and e) guide RNA comprising a tracrRNA having a sequence that binds to the Cas nuclease and a CRISPR RNA (crRNA) having a short “guide” sequence that is complementary to and binds to a specific target cleavage site in the genomic DNA that is proximal to the mutation, and f) a donor oligodeoxynucleotide (donor ODN) or donor DNA template that has a central region that is complementary to the desired corrected sequence for the mutation region in the genomic DNA, and flanking regions on each side of the central region that are complementary to and abut the sequences on each side of the desired corrected sequence for the mutation, wherein the donor oligodeoxynucleotide (donor ODN) or donor DNA template is configured to replace the disease/condition-causing mutation region in the genomic DNA by homology-directed repair (HDR) after the genomic DNA is cleaved by the CRISPR-Cas nuclease at the target cleavage site.

In certain embodiments, the cells are selected from the group consisting of: i) blood cells, hematopoietic stem cells (HSCs), iii) hemopoietic progenitor cells (HPCs), iv) induced pluripotent stem cells (iPSCs), v) a mixture of HSCs and HPCs (HSPCs), vi) cord blood HSCs, vii) T cells, viii) natural killer cells, ix) dendritic cells, and x) any cells derived therefrom. In particular embodiments, the first protein is a human mutant p53 tumor suppressor protein (mutant TP53), wherein the mutant TP53 protein lacks all or substantially all of the transactivation domains, the proline-rich region, and the DNA binding domain, but comprises all or substantially all of each of the tetramerization domain and the regulatory domain, thus enabling oligomerization of the mutant TP53 with wild-type TP53 proteins in the cell to form defective tetrameric proteins that results in a dominant negative inhibitory effect on the TP53 activity in the cell. In other embodiments, the first protein is a human mutant p53 tumor suppressor protein (mutant TP53) wherein the mutant TP53 lacks and does not comprise: all of the amino acids comprising the DNA binding domain or wild-type TP53, thus preventing the mutant TP53 protein to bind to genomic DNA in the cell to function as a transcription factor, plus; all or substantially all of the amino acids comprising the proline rich region and the two transactivation domains of wild-type human TP53 protein, thus preventing the mutant TP53 protein from transactivating other genes that would be transactivated by wild-type TP53; and wherein the mutant TP53 does comprise: an N-terminal methionine (M) and either aspartic acid (D) or glutamic acid (E) vicinal to the N-terminal M, plus: all or substantially all of the amino acids comprising both the tetramerization (or oligomerization) domain and the regulatory domain of wild-type human TP53 protein, thus enabling oligomerization of the mutant TP53 with wild-type TP53 proteins in the cell to form defective tetrameric proteins that results in a dominant negative inhibitory effect on the TP53 activity in the cell.

In further embodiments, the first protein in 1.b) is selected from the group consisting of: i) GSE CS-56; ii) CS TP53DD; iii) a deletion mutant of the 393-amino acid human wild type TP53 protein wherein the amino acids at positions 1 and 2 are M and D or M and E and all of the amino acids at positions 3-276 are deleted and the wild-type amino acids at positions 277-393 are present; and iv) a deletion mutant of the 393-amino acid human wild type TP53 protein wherein the amino acids at positions 1 and 2 are M and D or M and E and all of the amino acids at positions 3-292 are deleted and the wild-type amino acids at positions 293-393 are present.

In further embodiments, the third mRNA encodes a CRISPR/Cas protein selected from the group consisting of Cas9, Cas9 nickase, Casl2a, Cas14, and a Type V Cas endonuclease. In some embodiments, the mRNAs are in vitro-synthesized modified mRNA molecules that are purified using a process that removes RNA contaminant molecules that are immunogenic and toxic to the cell by inducing an innate immune response, as can be detected by measuring decreased secretion of IL-12, INF-alpha or TNF-alpha cytokine from monocyte-derived dendritic cells (MDDCs) transfected with the purified modified mRNA molecules compared to secretion of the cytokine from MDDCs transfected with the unpurified modified mRNA molecules, such that the purified RNA preparation is free of RNA contaminant molecules that, if present, would activate an immune response in the cell sufficient to prevent survival of the cell.

In some embodiments, provided herein are methods of generating a gene-edited stem or progenitor cell comprising: a) contacting an initial cell from a subject with first, second, third, fourth, and fifth reagents in vitro, wherein the initial cell comprises a mutation in its genome that is responsible for a disease or condition in a subject from which cell was derived, wherein the first reagent comprises a first mRNA encoding a first protein, wherein the first protein is capable of reducing, suppressing or preventing an innate immune response in the initial stem or progenitor cell, wherein the second reagent comprises a second mRNA encoding a second protein that inhibits 53BPI, wherein the third reagent comprises a third mRNA encoding a third protein, wherein the third protein comprises a Cas nuclease for use in CRISPR, wherein the fourth reagent comprises a guide RNA (gRNA or sgRNA) comprising a tracrRNA having a sequence that binds to a Cas enzyme and a CRISPR RNA (crRNA) having a short “guide” sequence that is complementary to and binds to a specific target cleavage site in the genome that is near to the mutation, and wherein the fifth reagent comprises a donor oligodeoxynucleotide (donor ODN), wherein the ODN comprises two outer flanking regions that are complementary to one strand of the genomic DNA on each side of the mutation site region in the genomic DNA and a middle region of interest, wherein the region of interest is configured to replace the disease/condition-causing mutation region in the genomic DNA by homology-directed repair (HDR) after the genomic DNA is cleaved by the CRISPR-Cas nuclease at the target cleavage site, wherein the contacting is under conditions such that the region of interest replaces the disease/condition-causing mutation region in the genomic DNA, thereby generating a gene-edited cell.

In certain embodiments, the contacting comprises: i) first contacting the cell with the first, second, third, fourth reagents and fifth reagents that comprise nucleic acids, and ii) subsequently treating the cell with the second reagent comprising a small molecule. In other embodiments, the contacting comprises: i) electroporating the cell in a solution comprising the first, second, third, and fourth reagents, and ii) subsequently treating the cell with a vector encoding the fifth reagent.

In some embodiments, the methods and compositions and systems further comprise comprising glycerol as a sixth reagent, wherein the contacting the cell includes contacting with the sixth reagent. In other embodiments, the region of interest replaces the disease/condition-causing mutation region during homology-directed repair (HDR) after the Cas nuclease cleaves the genomic DNA at the target site.

In additional embodiments, the methods further comprise: administering the gene-edited cell to the subject. In certain embodiments, the administering comprises intravenous administration to the subject. In other embodiments, the administering comprises intra-bone delivery of the gene-edited cell in the subject. In further embodiments, the methods further comprise: mixing the gene-edited cell with a physiologically tolerable buffer to generate a composition for treating the subject. In additional embodiments, the methods further comprise: b) shipping the gene-edited cell to a healthy care facility where the subject is, or where the subject is to be treated.

In some embodiments, the methods further comprise: prior to step a), the step of obtaining the initial cells from the subject, and isolating the initial cells. In additional embodiments, the isolating comprises: i) separating the initial cell from a blood sample from the subject, or ii) separating the cell from a bone marrow sample from the subject. In further embodiments, the gene-edited cell is free from off-target mutations. In some embodiments, the methods further comprise: culturing the gene-edited cell for a plurality of days.

In particular embodiments, the guide RNA comprises tracrRNA and crRNA as separate molecules, (collectively “gRNA”) or joined together to form single guide RNA (sgRNA). In other embodiments, the systems and composition further comprise: a donor oligodeoxynucleotide (donor ODN) or donor DNA template that has a central region that is complementary to the desired corrected sequence for the mutation region in the genomic DNA, and flanking regions on each side of the central region that are complementary to and abut the sequences on each side of the desired corrected sequence for the mutation, wherein the donor oligodeoxynucleotide (donor ODN) or donor DNA template is configured to replace the disease/condition-causing mutation region in the genomic DNA by homology-directed repair (HDR) after the genomic DNA is cleaved by the CRISPR-Cas nuclease at the target cleavage site.

In certain embodiments, the first protein is a human mutant p53 tumor suppressor protein (mutant TP53), wherein the mutant TP53 protein lacks all or substantially all of the transactivation domains, the proline-rich region, and the DNA binding domain, but comprises all or substantially all of each of the tetramerization domain and the regulatory domain, thus enabling oligomerization of the mutant TP53 with wild-type TP53 proteins in the cell to form defective tetrameric proteins that results in a dominant negative inhibitory effect on the TP53 activity in the cell. In other embodiments, the first protein is GSE CS-56 or CS TP53DD.

In particular embodiments, the systems and compositions further comprise: at least one of the following: (g-i)glycerol, wherein the final percent concentration of the glycerol in the composition or system is selected from: 2%, 3%, 4%, 5%, 6%, 7%, 8%, 9% and 10%, wherein the glycerol results in at least one of the following benefits: increased viability of the cells during electroporation; increased rates of survival of the gene-edited cells that have on-target corrections of the mutation due the gene editing; and/or increased rates of transplantation of HSPCs the gene-edited cells that have on-target corrections of the mutation due the gene editing in vivo; or (g-ii) trimethylglycine (aka glycine betaine or betaine), wherein the final percent concentration of the glycine betaine in the composition or system is selected from: 1-5%, 5-10%, 10-15%, 15-20%, and 20-25%, wherein the trimethylglycine results in at least one of the following benefits: increased viability of the cells during electroporation; increased rates of survival of the gene-edited cells that have on-target corrections of the mutation due the gene editing; and/or increased rates of transplantation of HSPCs the gene-edited cells that have on-target corrections of the mutation due the gene editing in vivo; or dimethylsulfoxide DMSO), wherein the final percent concentration of the DMSO in the composition or system is selected from: 0.1-1%, 1%-5%, 5-10%, 10-15%, 15-20%, 20-25%, 25-30%; and 30-35%; wherein the DMSO results in at least one of the following benefits: increased viability of the cells during electroporation; increased rates of survival of the gene-edited cells that have on-target corrections of the mutation due the gene editing; and/or increased rates of transplantation of HSPCs the gene-edited cells that have on-target corrections of the mutation due the gene editing in vivo.

In certain embodiments, the systems and compositions further comprise: a cell cycle modulator that inhibits microtubule polymerization, arresting cells in the G2/M phase of the cell cycle, selected from the group consisting of ABT-751, and Nocodazole. In other embodiments, the in vitro-synthesized modified mRNA molecules are purified using a process that removes RNA contaminant molecules that are immunogenic and toxic to the cell by inducing an innate immune response, as can be detected by measuring decreased secretion of IL-12, INF-alpha or TNF-alpha cytokine from monocyte-derived dendritic cells (MDDCs) transfected with the purified modified mRNA molecules compared to secretion of the cytokine from MDDCs transfected with the unpurified modified mRNA molecules, such that the purified RNA preparation is free of RNA contaminant molecules that, if present, would activate an immune response in the cell sufficient to prevent survival of the cell.

In particular embodiments, the cells are human cells and comprise at least one of the following: a hematopoietic stem cell (HSC), a hemopoietic progenitor cell (HPC), an induced pluripotent stem cell (iPSC), a mixture of HSCs and HPCs (HSPCs), a T cell, or a cord blood HSC. In other embodiments, the first protein comprises: a p53 protein (TP53) inhibitor. In additional embodiments, the TP53 inhibitor comprises: i) a TP53 variant protein comprising one or more mutations that inhibit wild-type TP53 expression, ii) pifithrin-alpha, iii) Pifithrin-α hydrobromide; and iv) Cyclic Pifithrin-α hydrobromide. In certain embodiments, the TP53 variant protein is selected from the group consisting of: GSE56, GSE-CS-19, GSE CS-56, TP53DD.

In some embodiments, the second protein comprises a ubiquitin variant comprising a variant of SEQ ID NO:1 with an amino acid modification at one or more of the following positions: 2, 4, 6, 8, 10, 11, 12, 14, 44, 46, 47, 48, 49, 62, 63, 64, 66, 68, 69, 70, 71, 72, 73, 74, 75, and 76. In other embodiments, the second protein comprises a ubiquitin variant comprising the amino acid sequence in SEQ ID NO:2 (“i53”) or a variant of SEQ ID NO:2 that comprises at least 60 consecutive aminos acids from SEQ ID NO:2, or has at least 95% sequence identity with SEQ ID NO:2.

In particular embodiments, the disease/condition-causing mutation region is: i) a single nucleotide polymorphism, or ii) a stretch of 2-35 nucleotides that differ from the corresponding non-diseased wild-type, or iii) part of, or an entire exon. In other embodiments, the cell is a T-cell, stem cell, or progenitor cell. In further embodiments, the cell is a hematopoietic stem cell (HSC). In further embodiments, the cell is a bone-marrow HSC.

In additional embodiments, the mRNAs encoding the first, second, and third proteins comprise at least one modified nucleoside selected from the group consisting of: pseudouridine (Ψ), 1-methylpseudouridine (m¹Ψ), 5-methyluridine (m⁵U), 5-methoxyuridine (mo⁵U), 2′-O-methyluridine (Um or m^{2′-O}U), and 2-thiouridine (s²U) in place of uridine, wherein the modified mRNAs comprising the modified nucleoside are significantly less immunogenic than the counterpart mRNA that does not comprise the modified nucleoside in place of uridine; and 5-methylcytidine (m⁵C) in place unmodified nucleoside cytidine; or N⁶-methyladenosine (m⁶A) in place of unmodified adenosine. In certain embodiments, all or most of the uridines in the first, second, and third mRNAs are replaced with pseudouridine (Ψ), 1-methylpseudouridine (m¹Ψ), 5-methyluridine (m⁵U), 5-methoxyuridine (mo⁵U). In some embodiments, all or most of the cytidines in the first, second, or third mRNA are replaced with 5-methylcytidine (m C).

In further embodiments, the systems and compositions further comprise: a first aqueous solution, wherein the mRNAs encoding the first, second, and third proteins are present in the first aqueous solution and are in vitro-transcribed mRNA. In additional embodiments, the first aqueous solution is detectably free of RNA contaminant molecules that, if present, would activate an immune response in the stem or progenitor cell sufficient to prevent survival of the cell. In some embodiments, the cell is an induced pluripotent stem cell (iPSC). In additional embodiments, the mRNAs encoding the first, second, and third proteins exhibit a 5′-cap and a 3′ poly(A) tail. In certain embodiments, the systems and compositions further comprise glycerol.

In additional embodiments, the Cas nuclease comprises a wild-type Cas9 nuclease, or wherein the mRNA encoding the Cas nuclease is modified such that no RNase III cleavage sites are present. In certain embodiments, the disease/condition-causing mutation region is associated with a gene, disease, or condition selected from the group consisting of: MAGT1 gene, XMEN disease, cancer, blindness, muscular dystrophy, Huntington's disease, eta-thalassemia, sickle cell disease, AIDS, cystic fibrosis, hemophilia, X-SCID, chronic granulomatous disease (CGD), CYBB, CTLA4 Deficiency, T−/B+ SCID (γc deficiency, JAK3 deficiency, interleukin 7 receptor chain α deficiency, CD45 deficiency, CD3δ/CD3ε deficiency), T−/B− SCID (RAG 1/2 deficiency, DCLRE1C deficiency, adenosine deaminase (ADA) deficiency, reticular dysgenesis), Omenn syndrome, DNA ligase type IV deficiency, Cernunnos deficiency, CD40 ligand deficiency, CD40 deficiency, Purine nucleoside phosphorylase (PNP) deficiency, CD3γ deficiency, CD8 deficiency, 1ZAP-70 deficiency, Ca++ channel deficiency, MHC class I deficiency, MHC class II deficiency, Winged helix deficiency, CD25 deficiency, STAT5b deficiency, Itk deficiency, DOCK8 deficiency, Activated PI3K Delta Syndrome, MALT1 deficiency, BCL10 deficiency, CARD11 deficiency, X-linked agammaglobulinemia (btk deficiency, or Bruton's agammaglobulinemia), μ-Heavy chain deficiency, 1 5 deficiency, Iga deficiency, BLNK deficiency, thymoma with immunodeficiency, common variable immunodeficiency (CVID), ICOS deficiency, CD19 deficiency, TACI (TNFRSF13B) deficiency, BAFF receptor deficiency, Hyper-IgM syndromes, heavy chain deletions, kappa chain deficiency, isolated IgG subclass deficiency, IgA with IgG subclass deficiency, selective immunoglobulin A deficiency, Transient hypogammaglobulinemia of infancy (THI), Wiskott-Aldrich syndrome, ataxia-telangiectasia, ataxia-like syndrome, Nijmegen breakage syndrome, Bloom syndrome, DiGeorge syndrome (when associated with thymic defects), cartilage-hair hypoplasia, Schimke syndrome, Hermansky-Pudlak syndrome type 2, Hyper-IgE syndrome, chronic mucocutaneous candidiasis, hepatic venoocclusive disease with immunodeficiency (VODI), XL-dyskeratosis congenita (Hoyeraal-Hreidarsson syndrome), Ch6diak-Higashi syndrome, Griscelli syndrome type 2, perforin deficiency, UNC13D deficiency, syntaxin 11 deficiency, X-linked lymphoproliferative syndrome, Autoimmune lymphoproliferative syndrome: type 1a (CD95 defects), type 1b (Fas ligand defects), type 2a (CASP10 defects), type 2b (CASP8 defects); APECED (autoimmune polyendocrinopathy with candidiasis and ectodermal dystrophy); IPEX (immunodysregulation polyendocrinopathy enteropathy X-linked syndrome); CD25 deficiency, severe Congenital Neutropenia: due to ELA2 deficiency (with myelodysplasia), Severe Congenital Neutropenia: due to GFI1 deficiency (with T/B lymphopenia), Kostmann syndrome, Neutropenia with cardiac and urogenital malformations, Glycogen storage disease type 1b, Cyclic neutropenia, X-linked neutropenia/myelodysplasia, P14 deficiency, Leukocyte adhesion deficiency type 1, Leukocyte adhesion deficiency type 2, Leukocyte adhesion deficiency type 3, RAC2 deficiency (Neutrophil immunodeficiency syndrome), Beta-actin deficiency, Localized juvenile periodontitis, Papillon-Lefèvre syndrome, Specific granule deficiency, Shwachman-Diamond syndrome, Chronic granulomatous disease: X-linked, Chronic granulomatous disease: autosomal (CYBA), Chronic granulomatous disease (CGD): autosomal (NCF1), Chronic granulomatous disease: autosomal (NCF2), IL-12 and IL-23 β1 chain deficiency, IL-12p40 deficiency, 2Interferon γ receptor 1 deficiency, Interferon γ receptor 2 deficiency, STAT1 deficiency (2 forms), AD hyper-IgE, 2AR hyper-IgE, pulmonary alveolar proteinosis, Hypohidrotic ectodermal dysplasia (NEMO deficiency, IKBA deficiency); EDA-ID, IRAK-4 deficiency, MyD88 deficiency, Epidermodysplasia verruciformis, Herpes simplex encephalitis, chronic mucocutaneous candidiasis, Trypanosomiasis, Familial Mediterranean fever, TNF receptor associated periodic syndrome (TRAPS), Hyper-IgD syndrome (HIDS), CIAS1-related diseases (Muckle-Wells syndrome, Familial cold autoinflammatory syndrome, Neonatal onset multisystem inflammatory disease), PAPA syndrome (pyogenic sterile arthritis, pyoderma gangrenosum, acne), Blau syndrome, Chronic recurrent multifocal osteomyelitis and congenital dyserythropoietic anemia (Majeed syndrome), DIRA (deficiency of the IL-1 receptor antagonist), C1q deficiency (lupus-like syndrome, rheumatoid disease, infections), C1r deficiency (idem), C1s deficiency, C4 deficiency (lupus-like syndrome), C2 deficiency (lupus-like syndrome, vasculitis, polymyositis, pyogenic infections), C3 deficiency (recurrent pyogenic infections), C5 deficiency (Neisserial infections, SLE), C6 deficiency (idem), C7 deficiency (idem, vasculitis), C8a deficiency, C8b deficiency, C9 deficiency (Neisserial infections), C1-inhibitor deficiency (hereditary angioedema), Factor I deficiency (pyogenic infections), Factor H deficiency (haemolytic-uraemic syndrome, membranoproliferative glomerulonephritis), Factor D deficiency (Neisserial infections), Properdin deficiency (Neisserial infections), MBP deficiency (pyogenic infections), MASP2 deficiency, Complement receptor 3 (CR3) deficiency, Membrane cofactor protein (CD46) deficiency, Membrane attack complex inhibitor (CD59) deficiency, Paroxysmal nocturnal hemoglobinuria, or Immunodeficiency associated with ficolin 3 deficiency.

In particular embodiments, the systems and compositions further comprise: the human or animal cell. In other embodiments, the human or animal cells is from a subject with a Primary Immunodeficiency Disease (PID). In some embodiments, the PID is: i) a monogenic PID; ii) a phagocytic disorder; iii) a chronic granulomatous disease (CGD); or iv) a lymphocytic disorder. In certain embodiments, the Cas endonuclease is selected from the group consisting of: CRISPR Cas9, CRISPR/Cas9 nickase, Cas12a, Cas14, and a Type V Cas endonuclease.

In certain embodiments, the mutation is in a target gene encodings one of the several NADPH oxidase subunits, such gp91phox, p47phox, p67phox, p22phox, and p40phox, each of which mutations results in a different form of chronic granulomatous disease (CGD). In certain embodiments, the mutation is one of a number of mutations identified in the target gene CTLA4 that encodes cytotoxic T-lymphocyte-associated protein 4. In certain embodiments, the mutation is one of the mutations in the target gene MAGT1, which encodes the magnesium protein MagT1. In other embodiments the mutation is in a gene encoding in another protein listed in Table 3. In certain embodiments, the cell is a T-cell, stem cell (hematopoietic stem cell), or hematopoietic progenitor cell from a subject with the disease or condition. In some embodiments, the cells that are edited are induced pluripotent stem cells generated by reprogramming a human somatic cell obtained from a subject with the disease or condition. In some embodiments, the cells that are edited are hematopoietic stems or progenitor cells generated by differentiation of induced pluripotent stem cells (iPSCs) from a subject with the disease or condition. In some embodiments, the cells that are edited are hematopoietic stems or progenitor cells generated by differentiation of induced pluripotent stem cells from a subject with the disease or condition. In some embodiments, the cells that are edited are HSCs from cord blood or cord blood stem cells. In some embodiments, the cells that are edited are adult stem cells selected from the group consisting of hematopoietic stem cells (HSCs), mesenchymal stem cells (MSCs), neural stem cells (NSCs) and epithelial stem cells (ESCs). In some embodiments, the cells that are edited comprise adult progenitor cells selected from the group consisting of hematopoietic progenitor cells (HPCs), mesenchymal progenitor cells (MPCs), neural stem cells (NPCs) and epithelial stem cells (EPCs). In some embodiments, the cells that are edited are differentiated cells. In some embodiments, the cells that are edited comprise cells that are further differentiated such as lymphocytes or vascular endothelial cells. In some embodiments, the cells that are edited comprise leukocytes. In some embodiments, the cells that are edited comprise T cells. In some embodiments, the cells that are edited comprise one or more other types of immune cells.

The present application describes compositions, systems, kits, methods and uses thereof for use in improving the efficiency of correction, the rates cell viability and survival, and the number of cells successfully transplanted following CRISPR/Cas gene editing of HSPCs or other cells obtained from or derived from cells from patients with primary immune deficiencies due to condition-causing mutations.

These compositions, systems, kits, methods and uses thereof are, in some embodiments, intended for improving CRISPR/Cas gene editing by homology driven repair (HDR), regardless of whether the disease-causing mutations comprise point mutations or indels or certain other mutations that can be handled using a donor oligodeoxynucleotide (ODN) or a recombinant adeno associated virus (rAAV) vector to deliver the donor template to the mutation site region for HDR.

In particular embodiments, the CRISPR/Cas nuclease is encoded by a highly purified modified-nucleoside-containing mRNA that is introduced into the cell by electroporation of the HSPC or other cell that is gene-edited along with the sgRNA, and the donor ODN that comprises the sequence that will repair the mutation site region (unless a rAAV virus is used to deliver the donor template).

In additional embodiments, two other highly purified modified nucleoside-containing mRNAs are introduced into the cell during the same electroporation. One of those mRNAs encodes human a TP53 protein that lacks the transactivation domains, the proline-rich region, and all of the DNA binding domain, but comprises all of the tetramerization and all or substantially all of the regulatory domain, thus resulting in a dominant negative effect when it tetramerizes with wild-type TP53 oligomers.

In one embodiment, the other mRNA encodes a protein discovered by Canny et al. (Nature Biotechnol., 36, 95, 2018) that binds to the tandem tudor domain (TTD) of p53 binding protein 1 (53BP1) at the same site where the 53BP1 TTD would bind to the dimethylated lysine20 on the N-terminal tail of histone 4 (H4K20me2), thereby initiating a double-stranded-break (DSB) response that ultimately leads to the DNA repair by non-homologous end joining (NHEJ) by DNA ligase IV. Thus, by blocking binding of the tudor domain of 53BP1 to H4K20me2 of histone 4, i53 protein inhibits repair by NHEJ and increase repair by HDR, which is less error prone, by about 2-fold.

A Fradet-Turcotte et al. (Nature 499(7456), 50, 2013) found that 53BP1 also recognized DNA damage-induced histone H2A that was ubiquitylated on Lys15 (H2AK15ub), which they referred to as ubiquitylation-dependent recruitment (UDR). In some embodiments, the gene editing compositions, systems, kits, methods, and uses thereof of the present invention comprise a small molecule inhibitor or a highly purified modified mRNA that encodes a protein that binds to the UDR domain of 53BP1 at the site where it binds to the H2AK15ub site of histone H2A, thereby blocking the ubiquitylation-dependent recruitment (UDR) motif of 53BP1 that interacts with the epitope formed by H2AK15ub and its surrounding residues on the tail of histone H2A, which may further inhibit NHEJ.

In other embodiments of the gene editing compositions, systems, kits, methods, and uses thereof of the present invention is a small molecule, selected from glycerol, trimethylglycine and dimethylsulfoxide that is added prior to electroporation to reduce damage to cells and promote cell recovery during and after electroporation.

In certain embodiments, the mRNAs used in compositions, systems, kits, methods, and uses thereof of the present invention comprise a 5′ cap with a cap1 structure, contain a modified nucleoside in place of all of the uridine moieties, and a long (>175 A residues) poly(A) tail, and have been purified using methods that remove contaminants that are immunogenic and toxic by inducing an innate immune response. The low immunogenicity is observed in use by high translation due to no transcription inhibition, high viability due to lack of toxicity or lack of induction of pro-inflammatory or inflammatory cytokines, as can also be detected by measuring little or no secretion of IFN-alpha, TNF-alpha or IL-12 during a 24-hour period after transfecting monocyte-derived dendritic cells with said mRNAs, which levels of secretion are significantly lower than counterpart mRNAs comprising not comprising modified nucleoside in place or uridine, higher proportions of uncapped RNA molecules, short poly(A) tails or less purified RNAs. Applicants have not observed any significant upregulation of interferon or other cytokines or cellular stress genes, transcriptional repression, or significant toxicity or loss of viability in HSPCs in response to the mRNAs encoding Spy Cas9 nuclease, GSE-CS-56 TP53inhibitor, or i53 protein during gene editing.

The Cas9 mRNA and other mRNAs manufactured by the present applicants did not upregulate interferon genes or interferon-inducible genes above electroporation alone response, did not dramatically repress transcription, or dramatically decrease viability and colony-forming ability (survival) with respect to CRISPR/Cas9-AAV6 genome editing of HSPCs. Thus, Applicants observations were dramatically different than those reported by MK Cromer et al. (Molecular Therapy, 26, 2431, 2018) who observed that: “the global transcription repression appeared to be unique to Cas9 mRNA treatments” and “The dramatic downregulation of cell cycle-related GO processes [Gene Ontology processes] was observed only in Cas9 mRNA-treated samples, which appears to have occurred concurrently with the global metabolic and transcriptional repression unique to these samples.” Cromer et al. further reports: “One of the most striking results was the dramatic upregulation of interferon-related genes when Cas9 mRNA was delivered to cells. In Cas9 mRNA-treated samples, we observed significant upregulation of the interferon regulatory factor gene family (IRF1, IRF7, and IRF9) DDX58 (also known as RIG-I) that is known to induce the interferon response, [REF=Loo, Y M and Gale, M, Jr., Immunity, 34, 680-692, 2011] as well as interferons themselves (IFI6, IFI16, IFNB1) (Table S1). This is not surprising, since exogenous mRNAs have been shown to cause an interferon response in CD34+ HSPCs, [REF=G Schiroli et al., Sci. Transl. Med. 9, eaan0820, 2017] with particular upregulation of DDX58 and IRF7. Consistent with these findings, we also observed significant downstream effects of this response within genes that are reported to be induced by interferons, such as the 20-50-oligoadenylate synthetase gene family (OAS1, OAS2, OAS3, and OASL), [REF=AJ Sadler & BRG Williams, Nature Rev. Immunol. 8, 559-568, 2008] the interferon-inducible gene family (IFI44L, IFIH1, IFIT1, IFIT2, IFIT5, IFITMI, and IFITM3), as well as other interferon-inducible genes such as MX1 and RSAD2, all of which displayed significant upregulation when cells were exposed to Cas9 mRNA.” Cromer et al. further reports “we performed an identical experiment and also found that delivery of Cas9 in the form of mRNA decreased viability as well as colony forming ability by approximately 15% compared to Cas9 in the form of RNP (FIGS. S11A and S11B).”

In some embodiments, the subject has a PID and an infection, and the systems and methods herein reduce the infection in the subject with PID, such as reduce a sign or symptom of a bacterial, viral, fungal, or parasitic infection, such as one or more of a fever, swelling, redness, and pain. For example, the disclosed methods can reduce infection in a subject, such as reduce viral load, by at least 20%, at least 30%, at least 40%, at least 50%, at least 75%, at least 80%, at least 90%, at least 95%, at least 98%, at least 99%, or even 100%, (such a reduction of 20% to 50%, 40% to 50%, 20 to 75%, 40% to 60%, or 20% to 90%), for example as compared to no treatment with the disclosed methods (e.g., prior to treatment with the disclosed methods).

### Definitions

As used herein, the terms “host,” “subject” and “patient” refer to any animal, including but not limited to, human and non-human animals (e.g., dogs, cats, cows, horses, sheep, poultry, fish, etc.) that is studied, analyzed, tested, diagnosed or treated. As used herein, the terms “host,” “subject” and “patient” are used interchangeably, unless indicated otherwise. In certain embodiments, the subject is a human. In some examples, the subject has a PID, such as one of those listed in Table 3. In some examples, the subject has a PID and a chronic infection.

When used herein, “aka” is an abbreviation for “also known as,” meaning that a name is also known by the respective other name or names that are recited.

Chronic granulomatous disease (CGD): A diverse group of hereditary diseases in which certain cells of the immune system have difficulty forming the reactive oxygen compounds (e.g., the superoxide radical due to defective phagocyte NADPH oxidase) used to kill certain ingested pathogens. The severely reduced phagocyte NADPH oxidase activity in granulocytes results in CGD patients being at significant risk for morbidity and mortality due to serious infections and inflammatory complications. Patients with CGD have received surgery or even allogeneic stem cell transplants as treatments. In non CGD-patients, superoxide anion is transformed into a variety of microbiocidal and regulatory reactive oxygen species (ROS) such as hydrogen peroxide, hydroxyl anions, hypochlorous acid (bleach) and peroxynitrite. NADPH oxidase is expressed primarily in phagocytic granulocytes (neutrophils, monocytes, eosinophils) but also occurs in monocytes and macrophages. Microbes such as bacteria and fungi are normally engulfed by granulocytes and killed by NADPH oxidase-dependent ROS working along with granulocyte proteases, enzymes, and antimicrobial proteins and polypeptides. Granulocytes are, therefore, a defense against bacteria and fungi as evidenced by the significantly increased risk of infection during periods of neutropenia or dysfunction and the observation that subjects with CGD suffer from frequent serious infections in the absence of antimicrobial prophylaxis.

The disclosed methods can be used to treat CGD, by utilizing the compositions herein, including a donor oligonucleotide to correct errors in genes such as: CYBA (p22phox), CYBB (gp91phox), NCF1 (p47phox), NCF2 (p67phox), or NCF4 (p40phox)) to restore expression of the defective protein in granulocytes needed to form the superoxide radical to kill pathogens. Mutations in the CYBA (p22phox), CYBB (gp91phox), NCF1 (p47phox, a 47 kDa cytosolic subunit of neutrophil NADPH oxidase), NCF2 (p67phox, a 67 kDa cytosolic subunit of neutrophil NADPH oxidase), or NCF4 (p40phox, a 40 kDa cytosolic subunit of NADPH oxidase) gene can cause CGD. Thus, there are five types of CGD that are distinguished by the gene that is involved, wherein the proteins produced from the affected genes are subunits of NADPH oxidase. There are several different types of CGD including X-linked CGD (affected gene codes for gp91), autosomal recessive cytochrome b-negative CGD (affected gene CYBA codes for p22phox), autosomal recessive cytochrome b-positive CGD type I (affected gene NCF1 codes for p47phox), autosomal recessive cytochrome b-positive CGD type II (affected gene NCF2 codes for p67phox), and autosomal recessive CGD (affected NCF 4 gene encodes for p40phox) atypical granulomatous disease. People with CGD often experience much more serious and invasive infections with organisms that may not cause as severe disease in people with normal immune systems. In some cases, these organisms do cause disease in people with a normal immune system. Among the most common organisms that cause disease in CGD patients are: (1) bacteria (particularly those that are catalase-positive), such as Staphylococcus aureus, Serratia marcescens, Burkholderia cepacia complex, Listeria species, E. coli, Klebsiella species, Pseudomonas cepacia, Nocardia and (2) fungi, such as Aspergillus species (including Aspergillus fumigatus) and Candida species. Thus, in some examples, a subject treated using the methods provided herein is infected with one or more of these organisms.

Cytotoxic T-lymphocyte-associated protein 4 (CTLA4) deficiency: A primary immune deficiency caused by mutations in the CTLA4 gene, a crucial controller of immune responses. A lack of CTLA-4 results in autoimmune complications that include insulin-dependent diabetes mellitus, Graves's disease, Hashimoto's thyroiditis, and systemic lupus erythematosus. CTLA-4 sequences are publicly available, for example from the GenBank® sequence database (e.g., Accession Nos. AAL07473.1, AF34638.1, and AAF01489.1 provide exemplary CTLA-4 protein sequences; while Accession Nos. AF414120.1, AF220248.1 and NM_001003106.1 provide exemplary CTLA-4 nucleic acid sequences).

Cytochrome b-245 light chain (CYBA) (also known as p22-phox or p22phox): (e.g., OMIM 608508) A transmembrane protein that associates with NOX2, NOX1, NOX3 and NOX4 in a 1:1, and contributes to the maturation and the stabilization of the heterodimer that it forms with NOX enzymes (NOX1-4) in order to produce reactive oxygen species (ROS). The human CYBA gene is located at 16q24. Mutations in CYBA can cause CGD (e.g., loss of function of CYBA causes an absence of cytb). Mutations in the CYBA gene encoding p22phox are rare (about 6%) and lead to AR-CGD220. p22phox sequences are publicly available, for example from the GenBank® sequence database (e.g., Accession Nos. NP_000092.2, XP_523459.1, and XP_020949224.1 provide exemplary p22phox protein sequences; while Accession Nos. NM_000101.3, MUZQ01000150.1, and NM_024160.1 provide exemplary p22phox nucleic acid sequences).

Cytochrome b-245 heavy chain (CYBB) (also known as glycoprotein 91 (gp91) phox and NADPH oxidase 2 (Nox2)): (e.g., OMIM 300481). The enzymatic center of the NADPH oxidase, gp91phox, is encoded by an X-linked gene called CYBB. X-linked CGD is the most common form (about 70% of CGD patients) and is generally more severe than mutations in autosomally encoded subunits of the NADPH oxidase. CYBB is a heterodimer of the p91-phagocyte oxidase (phox) beta polypeptide (CYBB) and a smaller p22phox alpha polypeptide (CYBA). CYBB deficiency is one of five biochemical defects associated with CGD. The human CYBB gene maps to chromosome Xp21.1-p11.4. GP91phox sequences are publicly available, for example from the GenBank® sequence database (e.g., Accession Nos. NP_000388.2, NP_031833.3, and AFE71531.1 provide exemplary gp91phox protein sequences; while Accession Nos. NM_000397.3, NM_023965.1 and GAAH01000462.1 provide exemplary gp91phox nucleic acid sequences).

A “dominant negative mutation” is a mutation in a gene that results in an altered protein or other gene product that acts antagonistically to the wild-type allele of the gene or its wild-type protein or other gene product, such as by disrupting expression or activity. With respect to proteins that are encoded by polypeptides that oligomerize, a mutation in one of the polypeptides can be dominant negative mutation if it results in an inactive complex following oligomerization. For example, many dominant negative mutations have been reported in the gene for the tumor suppressor protein TP53, which often functions as a tetramer formed by binding of two dimers.

Granulocytes: White blood cell characterized by the presence of granules in their cytoplasm. They are also called polymorphonuclear leukocytes (PMN, PML, or PMNL) because of the varying shapes of the nucleus, which is usually lobed into three segments. There are four types of granulocytes: neutrophils, eosinophils, basophils, and mast cells.

“Hematopoietic stem and progenitor cells” (“HSPCs”) refer to a mixture of hematopoietic stem cells (HSCs) and “hematopoietic progenitor cells” (“HPCs”). HSCs are stem cells that can be isolated from the blood or bone marrow and that can self-renew and that can give rise to all of the many different specialized blood and immune cells. Unlike HSCs, HPCs lack significant self-renewing capacity, but HPCs can further differentiate into mature blood cells of all hematopoietic lineages, including myeloid and lymphoid lineages. Human HSPCs are often characterized by the CD34 protein, which is used as a marker to isolate them because all colony-forming activity of human bone marrow (BM) cells is found in the CD34+ fraction. Thus, they are often referred to as “CD34+ HSPCs.”

Leukocyte Adhesion Defect (LAD1): An autosomal recessive disorder resulting from mutations in ITGB2, which encodes the common CD18 subunit of the 1 integrins. The integrins are critical for the neutrophils to migrate into tissues to kill pathogens where infections occur. The human ITGB2 gene maps to chromosome 7:74, 777. In LAD patients, granulocytes are present in increased numbers, but are incapable of leaving the circulating blood to enter infection sites, resulting in uncontrolled infections in tissues. ITGB2/CD18 sequences are publicly available, for example from the GenBank® sequence database (e.g., Accession Nos. NP_000202.3, AAH05861.1, AAH99151.1, and NP_032430.2 provide exemplary CD18 protein sequences; while Accession Nos. NM_000211.4, MF374490.1, U13941.1 and NM_008404.4 provide exemplary CD18 nucleic acid sequences).

Magnesium transporter protein 1 (MAGT1): (e.g., OMIM 300715) A highly selective transporter for Mg2+. The human MAGT1 is a 70 kb gene that maps to Xq21.1. The MAGT1 protein serves as a magnesium-specific transporter and plays a role in magnesium homeostasis. MAGT1 is evolutionarily conserved and expressed in all mammalian cells with higher expression in hematopoietic lineages. MAGT1 sequences are publicly available, for example from the GenBank® sequence database (e.g., Accession Nos. Q9HOU3.1, NP_115497.4, AAY18812.1, XP_014983205.1, and XP_016799148.1 provide exemplary MAGT1 protein sequences; while Accession Nos. KR710974.1, DQ000005.1, XM_016943659.1 and XM_015127719.1 provide exemplary MAGT1 nucleic acid sequences).

Natural killer (NK) cells: A type of cytotoxic lymphocyte critical to the innate immune system. NK cells are large granular lymphocytes (LGL), and can differentiate and mature in the bone marrow, lymph nodes, spleen, tonsils, and thymus, where they then enter into the circulation.

Neutrophil cytosolic factor 1 (NCF1) (also known as p47phox and NOXO2): (e.g., OMIM 608512) A cytosolic protein that forms NADPH oxidase. The human NCF1 gene is located at 7q11.23. Mutations in NCF1 can cause CGD.

Neutrophil cytosolic factor 2 (NCF2) (also known as p67phox and NOXA2) (e.g., OMIM 608515) A cytosolic protein that forms NADPH oxidase. The human NCF2 gene is located at 125.3. Mutations in NCF2 can cause CGD.

Neutrophil cytosolic factor 4 (NCF4) (also known as p40phox) (e.g., OMIM 601488) A cytosolic protein that forms NADPH oxidase. The human NCF4 gene is located at 2212.3. Mutations in NCF4 can cause CGD.

P47phox sequences are publicly available, for example from the GenBank® sequence database (e.g., Accession Nos. NP_000256.4, DAA15017.1, and AAX08869.1 provide exemplary p47phox protein sequences; while Accession Nos. NM_000265.5, NM_174119.4, and NM_010876.4 provide exemplary p47phox nucleic acid sequences).

p67phox sequences are publicly available, for example from the GenBank® sequence database (e.g., Accession Nos. AAH01606.1, AFJ19027.1 and JAA02114.1 provide exemplary p67phox protein sequences; while Accession Nos. BC001606.1, JN864042.1, and AB002663.1 provide exemplary p67phox nucleic acid sequences).

P40phox sequences are publically available, for example from the GenBank® sequence database (e.g., Accession Nos. NP_000622.2, AAH25517.1, and NP_001120776.1 provide exemplary p40phox protein sequences; while Accession Nos. NM_000631.4, BC167076.1, and BT020852.1 provide exemplary p40phox nucleic acid sequences).

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of recombinant autologous granulocytes, recombinant autologous NK cells, and/or recombinant autologous lymphocytes that have been treated according to the methods, systems, and compositions herein (e.g., to correct a gene causing a PID). In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Primary immunodeficiency disease (PID): A group of disorders in which inherited defects in the immune system lead to increased infections, which can be associated with an increased risk of immune dysregulation and/or increased risk of developing cancer. There are currently more than 200 PIDs. Examples of infections in subjects with primary immunodeficiency diseases include infections that are unusually persistent, recurrent or resistant to treatment, infections involving unexpected spread or unusual organisms, and infections that are unexpectedly severe. PIDs, as well as infections in such subjects, can be treated with the disclosed methods, systems, and compositions. There are four general groups of PIDs. (1) antibody deficiencies, such as common variable immunodeficiency (CVID), and X-linked agammaglobulinaemia (e.g., are susceptible to certain viruses such as hepatitis and polio); (2) combined immunodeficiencies (subjects may lack T cells), such as X-linked Severe Combined Immunodeficiency (SCID); (3) complement deficiencies, such as C2 Deficiency (which can cause an autoimmune disease such as Systemic Lupus Erythematosus (SLE) or can result in severe infections such as meningitis) and hereditary angioedema (HAE) (due to C1 inhibitor deficiency); and (4) phagocytic cell deficiencies, such as CGD and LAD. Other specific examples of PIDs are provided in Table 3.

Therapeutically effective amount: The amount of agent, such as gene-edited HSCs, HPCs, HSPCs, iPSCs, or other cells derived therefrom, such as gene-edited autologous granulocytes autologous NK cells, and/or autologous lymphocytes (made by the systems, methods, and compositions herein), that is sufficient to prevent, treat, reduce and/or ameliorate the symptoms and/or underlying causes of a disorder or disease, such as a PID or a chronic infection in a PID patient. For example, it can be an amount of gene-edited HSCs, HPCs, HSPCs, iPSCs, or other cells derived therefrom, such as gene-edited autologous granulocytes, autologous NK cells, and/or autologous lymphocytes sufficient to improve immune system function in a treated subject, such as a subject having a PID, such as one of those listed in Table 3. An effective amount of gene-edited HSCs, HPCs, HSPCs, iPSCs, or other cells derived therefrom, such as gene-edited autologous granulocytes, autologous NK cells, and/or autologous lymphocytes can be determined by various methods, including generating an empirical dose-response curve, predicting potency and efficacy by using modeling, and other methods used in the art. In one embodiment, a therapeutically effective amount of gene-edited HSCs, HPCs, HSPCs, iPSCs, or other cells derived therefrom, such as gene-edited autologous granulocytes, autologous NK cells, and/or autologous lymphocytes is at least 1×10⁶, at least 5×10⁶, at least 1×10⁷, at least 5×10⁷, at least 1×10⁸, or at least 5×10⁸ gene-edited HSCs, HPCs, HSPCs, iPSCs, autologous granulocytes, autologous NK cells, and/or autologous lymphocytes. Specific assays for determining the therapeutically effective amount of gene-edited HSCs, HPCs, HSPCs, iPSCs, autologous granulocytes, autologous NK cells, and/or autologous lymphocytes are provided herein. For example, immune system function can be measured in the recipient subject.

Transfected: A cell is “transfected” when a nucleic acid molecule (such as mRNA) is introduced into the cell (such as a granulocyte, NK cell, or lymphocyte) and for example when the RNA becomes translated into the encoded protein without incorporation of the nucleic acid into the cellular genome. The process of introducing a nucleic acid, such as a DNA, RNA, or mRNA into a cell (e.g., during a process comprising CRISPR/Cas gene editing), is referred to as “transfection” and the method used is a “transfection method.” In some embodiments, the resulting cell after a transfection is a recombinant cell. “Transfected” and “transfection” and “transfection methods” encompass all techniques by which a nucleic acid molecule (such as an mRNA or DNA) can be introduced into a cell, including transfection with viral vectors, transformation with plasmid vectors, and introduction of nucleic acid molecules by electroporation, lipofection, lipid nanoparticles, transfection reagents, PEI, particle gun acceleration and other methods. In some example the method is a chemical method (e.g., calcium-phosphate transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994). In some embodiments of any of the any of the compositions, kits, systems, methods, or uses described in the present application, the nucleic acid is transfected into the cell by electroporation. For example, although the present invention could also be performed using a transfection reagent (e.g., an lipid nanoparticle (LNP) or other transfection reagent described herein), unless otherwise stated for a specific example, all examples described in the present document wherein a nucleic acid is introduced into a cell during CRISPR/Cas gene editing were transfected by electroporation, including:
(i) the nucleic acids comprising pseudouridine-modified mRNA, including:
   (i)(a) the mRNAs encoding GSE CS-56 and CS TP53DD, which are mutant of TP53 proteins that are dominant negative inhibitors of wild-type TP53 protein, and
   (i)(b) mRNA encoding i53 protein, which binds to a portion of p53 binding protein 1 (53BP1), thereby inhibiting the nonhomologous end joining (NHEJ) of dsDNA breaks in a cell that is undergoing CRISPR/Cas gene editing, thereby favoring the less error-prone homology directed repair (HDR) of said dsDNA breaks, and
   (i)(c) mRNA encoding the CRISPR/Cas nuclease enzyme used for gene editing), and
(ii) any oligodeoxynucleotide donors (ODNs) used during CRISPR/Cas gene editing to replace the region of the genomic DNA comprising the mutation site in the cell by HDR (including any single-stranded ODN or double-stranded ODN that is appropriate for the particular type of CRISPR/Cas enzyme and method used for said gene editing, preferably, wherein said donor ODN has a size that of at least 100 bases or basepairs (bp) and, more preferably, a size of ≥500 bases or bp, ≥1000 bases or bp, or larger), and
(iii) any guide RNA (gRNA) comprising a tracrRNA and a pre-crRNA, including a single guide RNA (sgRNA), that is appropriate for the specific CRISPR/Cas nuclease enzyme used for gene editing.

In many of the embodiments herein, the donor template is delivered within a recombinant adeno associated virus (AAV) that has been genetically engineered for such purpose, rather than as an ODN, in which case, the cell is transduced with the recombinant AAV that delivers the donor template for HDR during gene editing as described in the methods herein, rather than by electroporation when an ODN is used to deliver the donor to replace the mutation region.]

Transplantation: The transfer of a tissue or an organ, or cells (such as HSCs), from one body or part of the body to another body or part of the body. An “allogeneic transplantation” or a “heterologous transplantation” is transplantation from one individual to another, wherein the individuals have genes at one or more loci that are not identical in sequence in the two individuals. An allogeneic transplantation can occur between two individuals of the same species, who differ genetically, or between individuals of two different species. An “autologous transplantation” is a transplantation of a tissue or cells from one location to another in the same individual (such as removal of cells and subsequent reintroduction of the cells, which have been modified ex vivo (for example made recombinant Crisper medicated correction of Magt1, CTLA4, CD18, p47phox, p67phox, p22phox, p40phox or gp91phox mRNA, according to the methods herein), into the same subject), or transplantation of a tissue or cells from one individual to another, wherein the two individuals are genetically identical.

Treating, Treatment, and Therapy: Any measures taken to achieve attenuation or amelioration of an injury, pathology or condition, the abatement, remission, diminishing of symptoms or making of a condition more tolerable to the patient, slowing in the rate of degeneration or decline, making the final point of degeneration less debilitating, improving a subject's physical or mental well-being, or prolonging the length of survival, wherein success or indicia of success of the treatment may be assessed by objective or subjective parameters; including the results of a physical examination, blood and other clinical tests, and the like.

X-linked immunodeficiency with magnesium defect, Epstein-Barr virus infection, and neoplasia (XMEN): A genetic disorder that affects the immune system in males. In XMEN, T cells are reduced in number or do not function properly. Normally these cells recognize pathogens, such as viruses, bacteria, and fungi, and are activated to prevent infection and illness. Because males with XMEN do not have sufficient functional T cells, they have frequent infections, such as ear infections, sinus infections, pneumonia, and extremely high EBV viral loads. In particular, affected individuals are vulnerable to the Epstein-Barr virus (EBV). Many affected individuals also develop EBV-related lymphoproliferative disease. XMEN is caused by mutations in the MAGT1 gene, which encodes a magnesium transporter, which moves Mg2+ into T cells. Current management of these patients includes symptomatic control, administration of magnesium supplementation, and specific chemotherapy for lymphoproliferative disease. A few patients have been given allogenic stem cell transplant with fatal outcomes (Li et al., Blood 123:2148-2152, 2014; Ravell et al., Curr Opin Pediatr 26:713-719, 2014). The disclosed methods can be used to treat XMEN, by utilizing Crispr/Cas systems with a MAGT1 donor sequence to restore expression of NKG2D needed for antiviral immunity and clearance of transformed cells.

### DESCRIPTION OF THE DRAWINGS

The patent or application file contains at least one drawing executed in color. Copies of this patent or patent application publication with color drawings will be provided by the Office upon request and payment of the necessary fee.

FIGS. 1a and 1b—CYBB gp91 exon 7 C676 mutation-containing cells were CRISPR/Cas9 edited using an AAV-delivered, endogenous gp91 promoter-driven, GFP-expressing donor sequence also containing gp91 exons 7-13. Cas9, i53 and GSE CS-56 (aka GIS19 or GIS-019) were delivered as modified mRNAs. Percentage of live cells and GFP-expressing cells with and without i53 and GSE CS-56 mRNAs were assayed.

FIG. 2—CYBB gp91 exon 7 C676 mutation-containing cells were CRISPR/Cas9 edited using an AAV-delivered, endogenous gp91 promoter-driven, GFP-expressing donor sequence also containing gp91 exons 7-13. Cas9 and i53 were delivered as modified mRNAs. Percentage of GFP-expressing cells without and with increasing amounts of i53 mRNAs were assayed.

FIG. 3—Healthy donor cells were CRISPR/Cas9 edited using Cas9, i53 and GSEs delivered as modified mRNAs. AAV-delivered CD40L donor constructs were used in increasing amounts to elicit a DNA damage response. Cell proliferation was assayed.

FIG. 4—Healthy donor CD34+ hematopoietic stem cells were CRISPR/Cas9 edited using an AAV-delivered, endogenous gp91 promoter-driven, GFP-expressing donor sequence also containing gp91 exons 7-13. Cas9, i53 and GSE were delivered as modified mRNAs in the presence and absence of glycerol pre and post electroporation. Percentage of live cells and GFP-expressing cells with and without i53 mRNA, GSE mRNA and glycerol (pre and post electroporation) were assayed.

FIG. 5—CYBB gp91 exon 7 C676 mutation-containing cells were CRISPR/Cas9 edited using a 100-b single-stranded DNA donor of corrected sequence. Cas9 and i53 were delivered as modified mRNAs. Gp91 positive cells were assayed.

FIG. 6—CYBB gp91 exon 7 C676 mutation-containing cells were CRISPR/Cas9 edited using a 100-b single-stranded DNA donor of corrected sequence with Cas9 and i53 delivered as modified mRNAs. Resultant corrected cells were transplanted into immunodeficient mice and assayed for gp91 expression 16 weeks post transplantation.

FIG. 7—CYBB gp91 exon 7 C676 mutation-containing cells were CRISPR/Cas9 edited using an AAV-delivered donor sequence containing gp91 exons 7-13. Cas9 and i53 delivered as modified mRNAs. Targeted insertion rates were assayed by digital droplet PCR for samples using increasing amounts of i53.

FIGS. 8A-F—Two immunodeficiency gene models were CRISPR/Cas9 edited using AAV-delivered corrective donors targeted to the respective gene start sites. Cas9, i53 and GSE were delivered as modified mRNAs. 8a1/8a2) Targeted insertion rates into CD34+ cells were assayed by digital droplet PCR for samples in the presence or absence of i53, GSE and glycerol. 8b1/8b2) Cell viability was assayed 2 days post electroporation by trypan blue exclusion. 8c) hematopoietic stem cell differentiation into Natural Killer (NK) cells was assayed by flow cytometry 35 days post correction in model system 1 which normally lacks NK cells. 8d) NKG2D expression levels were assayed in the XMEN model system only for samples in the presence or absence of i53, GSE and glycerol. 8e1/8e2) Targeted insertion rates were assayed by digital droplet PCR for samples in the presence or absence of i53, GSE and glycerol. 8f1/8f2) T cell development in model system 1 was assayed in vitro using an artificial thymic organoid system 4 weeks and 6 weeks post correction. 8f3/8f4) IL-2 expression levels were assayed in model system 1 for samples in the presence or absence of i53, GSE and glycerol. 8f5) NKG2D expression levels were assayed in the XMEN model system only for samples in the presence or absence of i53, GSE and glycerol at 4 and 6 weeks post correction. 8f6/8f7) Cell viability was assayed at 4 weeks post correction.

FIGS. 9A-E—CRISPR/Cas9 edited CD34+ hematopoietic stem cells were transplanted into immunodeficient NSG mice. 9a1/9a2) Engraftment rates into bone marrow were assayed 16 weeks post transplantation by flow cytometry for hCD45+ cells for samples in the presence or absence of i53, GSE and glycerol. 9b1/9b2) Targeted insertion rates were assayed 16 weeks post transplantation by digital droplet PCR for samples in the presence or absence of i53, GSE and glycerol. 9c) Development of CD3+ T cells for model system 1 were assayed for samples in the presence of combinations of i53, GSE and glycerol. 9d1/9d2) STAT5 phosphorylation levels and IgG serum levels were assayed for model system 1 samples in the presence of combinations of i53, GSE and glycerol. 9e1/9e2) NKG2D expression levels in differentiated Natural Killer (NK) cells and CD8+ T cells were assayed in the XMEN system for samples in the presence of combinations of i53, GSE and glycerol.

FIG. 10—CYBB gp91 exon 7 C676 mutation-containing cells were CRISPR/Cas9 edited using an AAV-delivered, endogenous gp91 promoter-driven, GFP-expressing donor sequence also containing gp91 exons 7-13. Cas9 and i53 were delivered as modified mRNAs. Percentage of GFP-expressing cells with and without i53 and Eo were assayed 10 days and 17 days post correction.

FIG. 11A shows SEQ ID NO:3, which is the Phosphorothioate modified CYBB c676 correction oligo, used in correcting a single base mutation in CYBBgp91. In SEQ ID NO:3, the red and underlined G is the corrected nucleotide. FIG. 11B shows SEQ ID NO:4, which shows the i53 protein sequence (an example of a ubiquitin variant). Note this sequence differs from UbvG08 by one residue, the red and underline A, which is an I in UbvG08. Additionally, i53 is 2 residues shorter than UbvG08 which ends with GG residues (See, Canny et al., Nature Biotechnology 2018). FIG. 11C shows SEQ ID NO:5 which is the i53 ORF RNA sequence, where Y=pseudouridine). FIG. 11D shows SEQ ID NO:6, which is the IVT-RNA containing the i53 ORF RNA, where Y=pseudouridine. This IVT-RNA was in vitro transcribed from a linearized plasmid using T7 RNA polymerase with pseudouridine 5′-triphophate in place of 100% of the UTP. The plasmid this ORF was closed into was pCSilent-T7-XBG-22 (pCS22) which adds Xenopus beta-globin 5′ & 3′ UTRs, a Kozak consensus sequence and a 3′ restriction site for plasmid linearization. The i53 IVT-RNA was enzymatically capped to a cap1 structure and polyadenylated to yield a poly(A) tail with >175 As.

FIG. 12A shows SEQ ID NO:7, which is the amino acid sequence of GSE-CS56 (aka GIS19) which is dominant negative deletion mutant of the human wild-type p53 tumor suppressor gene or “TP53” gene. FIG. 12B shows SEQ ID NO:8, which is the ORF RNA sequence of GSE-CS56 (aka GIS19), where Y=pseudouridine. FIG. 12C shows SEQ ID NO:9, which is the in vitro transcribed sequence containing the GSE-CS56 (aka GIS19) ORF RNA, where Y=pseudouridine.

FIG. 13 shows protein and RNA sequences for Spy Cas9, which was engineered to alleviate transcript 5′-end secondary structural sequence to permit efficient enzymatic capping and remove inherent internal specific RNase III cleavage site in the transcript to permit RNase III treatment during the mRNA production process. FIG. 13a shows SEQ ID NO:10, which is the amino acid sequence of this Spy Cas9 enzyme. FIG. 13b shows SEQ ID NO:11, which is the Spy Cas9 ORF RNA sequence, where Y=pseudouridine. FIG. 13c shows SEQ ID NO:12, which is the IVT RNA Sequence containing the Spy Cas9 ORF.

FIG. 14 shows the Design of rAAV6-MAGT1 donor for CRISPR/Cas9 targeted integration into XMEN CD34+ HSPCs. (A) Location of the 10 candidate sgRNAs targeting MAGT1 gene transcription start site (TSS). Each sgRNA (sg) is represented as a 20-bp sequence (light grey or green) associated with the 3-bp PAM sequence (darker color). (B) Evaluation of the percentage of cutting activity by TIDE assay after gDNA extraction from untreated versus Cas9/sgRNA treated HD CD34+ cells at 2-5 days post electroporation, sequencing of Exon 1 and comparison of the sequences using TIDE software (n=1-5 independent experiments). (C) Design of the rAAV6-MAGT1 donor. (D) Molecular analysis showing the percentage of targeted insertion in gene-edited XMEN CD34+ cells using gene editing enhancers as described. gDNA was extracted at 48 h-72 h post-EP and insertion of the MAGT1 cDNA donor was quantified by ddPCR (n=3-11 independent experiments). (E) Viability determined by blue trypan exclusion in untreated Naive and AAV-MAGT1-treated XMEN HSPCs at day 2 post-EP (n=1-7 independent experiments). (F) Representative analysis of the phenotype of CD34+ cells at day 2 post-EP. For all the graphs, data are shown as mean±standard deviation (SD); ANOVA one-way test and Tukey's post hoc multiple comparisons test, *p<0.05, *p<0.01, ***p<0.001, ****p<0.0001.

FIG. 15A shows a vector map for rAAV6-MAGT1 AAV vector. FIG. 15B shows SEQ ID NO:13, which is the nucleic acid sequence of rAAV6-MAGT1. FIG. 15C shows the features of the various sequences in rAAV-MAGT1 with reference to SEQ ID NO:13.

FIG. 16 shows optimization of gene editing in HSPCs. (A) Optimization of cutting activity (% indels) of various forms of Cas9 mRNA and at increasing concentration. Commercial Cas9 mRNA (MaxCyte, Mx) is used as control. (B) Evaluation of the glycerol concentration in the EP mix on viability and cutting activity at day 2 post EP. (C) Representative dot plots showing the immunophenotype analysis of HSPCs by flow cytometry at day 2 post EP. (D) Hematopoietic potential evaluated by the CFU assay at day 2 post-EP; Number of CFU colonies were counted after 14 days. (E) Measure of the proliferation index after staining HD HSPCs at day 1 post EP with CFSE dye and analysis of the proliferation by flow cytometry at day 6 post EP.

FIG. 17 shows in vitro phenotypic and functional correction in immune cells after gene editing XMEN CD34+ HSPCs (A) Dot plots showing the gating strategy after in vitro T cell differentiation of CD34+ cells using the ATO system; histogram on the right showing the NKG2D expression in the CD3+ T cells at 6 weeks of differentiation (n=1-2 independent experiments). (B) Dot plot showing the gating for NK cells (CD3-CD56+) and NKG2D expression after 35 days of in vitro NK cell differentiation from CD34+ cells; histogram on the right showing the percentage of CD34-derived NK cells in each condition (n=2-8 independent experiments). (C) NKG2D expression and (D) targeted integration in NK cells at day 35 of in vitro differentiation (n=2-8 independent experiments). (E) Cytotoxic activity of CD34+-differentiated NK cells against K562 cells at an E:T ratio of 2:1 (n=1-7 independent experiments). For all the graphs, data are shown as mean±SD; *p<0.05, *p<0.01, ***p<0.001, ****p<0.0001. (F) Correlation between the NKG2D expression (%) and the killing activity (%) in NK cells at day 35 was calculated using Spearman's correlation coefficient and two-tailed p-value (n=21 samples).

FIG. 18 shows in vitro phenotypic and functional correction in immune cells after gene editing XMEN CD34⁺ HSPCs. (A) Representative histogram showing the NKG2D expression measured by flow cytometry in CD34⁺ HSPCs from HD (blue line; isotype control in light blue filled histogram), XMEN (red line) and AAV-treated XMEN (green line). (B) After 6 weeks of differentiation in the ATO system, cells were analyzed by flow cytometry as shown in the dot plot (right panel) and NKG2D expression was measured (left panel) in CD4⁺ (grey-filled histogram) and CD8⁺ cells (red-filled histogram) derived from HD HSPCs, and Naïve or gene-edited with AAV XMEN HSPCs. (C) Cytotoxic activity of CD34⁺-differentiated NK cells against K562 cells at increasing E:T ratios (left curve) (n=1-7 independent experiments).

FIG. 19 shows Hematopoietic reconstitution of gene-edited HSPCs in immunodeficient mice. (A) Human engraftment measured by the presence of human CD45⁺ cells by flow cytometry at week 16 in the peripheral blood, bone marrow, spleen and thymus of NSGS mice transplanted with HD (n=5 mice), untreated (n=12) or gene-edited XMEN CD34⁺ HSCs (n=1 (−i53), n=8 (+i53), n=18 (+i53⁺ GSE-CS56)). The dotted line indicates the threshold for engraftment at 0.2% hCD45⁺ cells in the bone marrow. (B) Percentage of human engraftment (hCD45⁺) measured after injection of XMEN HSPCs gene edited with increasing Multiplicity of Infection (MOIs) of AAV, in presence of i53 alone or i53 and GSE-CS56. (C) Immunophenotypic analysis of bone marrow at 16 weeks post-transplant by flow cytometry showing the percentages of CD34⁺ cells, myeloid (CD33⁺), lymphoid B (CD19⁺) and T (CD3⁺) after gating on the hCD45⁺ population. (D and E) Restoration of NKG2D expression in NK cells from the spleen (D) and CD8⁺ T cells from the peripheral blood, spleen and thymus (E) at week 16 post-transplant. For all the graphs, data are shown as mean±SD. (F and G) Targeted insertion quantified by ddPCR in human CD45⁺ cells isolated from the bone marrow after gene editing with i53 (orange bar) or with i53 and GSE-CS56 (green bar) (F); or in the lymphoid T (CD3), myeloid (CD33/CD14) and lymphoid B (CD19) cells sorted from the spleen (G).

FIG. 20 shows analysis of engraftment in immunodeficient mice. (A and B) In the blood, spleen and thymus harvested at week 16 post-transplant, we analyzed the relative proportion of CD3⁺ T cells (A) and NK cells (CD3-CD56⁺) (B) in the human CD45⁺ cells population. (C) Proportion of CD4⁺, CD8⁺ and double positive (DP) cells in the CD3⁺ T cell compartment from the blood, spleen and thymus at week 16 post-transplant in mice injected with HD cells, XMEN untreated (UT) and gene edited in presence of i53 alone or i53 and GSE-CS56. (D) Correlation between the percentage of human engraftment (hCD45⁺) and the percentage of targeted insertion in the bone marrow was calculated using Spearman's correlation coefficient and two-tailed p-value (n=42 samples).

FIG. 21 shows phenotypic correction in gene-edited XMEN T cells. (A) Representative blot showing MAGT1 protein expression at 48 h post-EP by Western Blot for HD and XMEN untreated (UT) and gene-edited T cells (0-actin for loading control). (B) Mean fluorescence intensity (MFI) of the expression of NKG2D measured in CD8⁺ T cells by flow cytometry at 2 days and 28 days post-EP (n=2).

FIG. 22 shows gene editing in XMEN T cells and analysis of off-target activity (A) Percentage of NKG2D-positive CD8⁺ T cells in HD, XMEN untreated (UT) and AAV-treated T cells at days 2 and 28 post-EP (n=2 independent experiments). (B) Expression of CD70 and CD28 by flow cytometry for HD, XMEN UT and gene-edited T cells at day 28 post-EP; data are expressed as the ratio of mean fluorescence intensity (MFI) normalized to XMEN untreated cells for CD70 and CD28 (n=2 independent experiments). (C) Targeted insertion of the AAV-donor quantified by ddPCR at day 28 post-EP (n=2 independent experiments).

FIG. 23 shows analysis of off-target activity. (A) Manhattan plot showing the results of off-targets for CD34⁺ cells from HD male donor by CHANGE-seq assay. The red arrow indicates the on-target site (MAGT1 gene) on chromosome X. (B) Table reporting the total number of cleavage sites (on- and off-targets) and the specificity ratio. (C) Pie charts showing fraction of cleavage sites categorized according to their genomic features after editing of CD34⁺ cells from HD male and female donors. (D) Identity of the nucleotide mismatches at the off-target sites. ON indicates the on-target site without mismatch while OT1-OT8 indicate the top eight OT sites shared by male and female HD CD34⁺ HSPCs and HD PBMCs. (E) Percentage of cutting activity evaluated by sequencing at on target (ON) and off-target (OT) sites detected by CHANGE-Seq in in vitro (CD34⁺ cells 2 days after gene editing) and in vivo samples (hCD45⁺ cells from the bone marrow of transplanted mice at week 16 post-transplant). For all the graphs, data are shown as mean±SD.

FIG. 24 shows the open reading frame of TP53DD DNA sequence (SEQ ID NO:45).

FIG. 25 shows a sequence alignment between human TP53, TP53DD, and GSE CS-56.

### DETAILED DESCRIPTION OF THE INVENTION

The present invention relates to compositions, systems, and methods for editing a disease/condition-causing mutation region in a target gene in a cell. In certain embodiments, the following components are employed: i) mRNA encoding a Tumor Protein p53 (TP53) inhibitor, ii) an inhibiting agent that inhibits Tumor Suppressor p53-Binding Protein 1 (53BPI) (e.g., small molecule EoHR or mRNA encoding a protein that inhibits 53BPI), iii) mRNA encoding a Cas nuclease for CRISPR; iv) a guide RNA specific for a target cleavage site proximal to said disease/condition-causing mutation region; and v) a repair template comprising a region of interest configured to replace said disease/condition-causing mutation region in the target gene during homology-directed repair (HDR). In certain embodiments, the cell is a T-cell, stem cell (e.g., hematopoietic stem cell), or hematopoietic progenitor cell from a subject with the disease or condition (e.g., a Primary Immunodeficiency Disease (PID)). In some embodiments, the gene-edited cell is administered to the subject.

Work was conducted during development of embodiments herein to functionally correct autologous MAGT1-deficient CD34⁺ HSC and progenitor cells (HSPCs) using the CRISPR/Cas9 genome editing (GE) approach with an Adeno-Associated Virus (AAV) vector to perform targeted insertion (TI) of a MAGT1 complementary DNA (cDNA) at the endogenous locus to restore physiological expression under the regulation of the endogenous promoter (14-17). To address reported problems with poor engrafting gene-edited HSPCs (18, 19), a barrier to clinical translation, we explored the addition of various agents to boost homology-directed repair (HDR) and cell fitness following GE (‘gene editing enhancers’ (GEE)). Using an mRNA encoding an inhibitor of 53BP1 (i53) to improve HDR and an mRNA encoding a human TP53 genetic suppressor element (GSE-CS56) that is a deletion mutant of human TP53 gene that results in a dominant negative phenotype to inhibit the p53-mediated DNA damage response during gene editing, we achieved highly efficient targeted integration rates in engrafting XMEN patient CD34⁺ HSPCs, which was maintained at 16 weeks after transplantation into NSGS immunodeficient mice. It was also shown that NKG2D receptor expression was successfully corrected in vitro and in vivo on NK and T cells derived from gene-edited XMEN HSPCs with restoration of NK cytotoxic activity. This versatile approach was facilely applied to correction of peripheral blood XMEN T cells which showed restoration of NKG2D expression and a survival advantage in culture. Functional gene correction with GEE of CD34⁺ HSPCs and peripheral blood T cells offers a two-pronged approach for more immediate protection in patients with acute infections, while latent T cell differentiation from engrafted GE CD34⁺ HSPCs for permanent correction.

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Each CRISPR locus encodes acquired “spacers” that are separated by repeat sequences. Transcription of a CRISPR locus produces a “pre-crRNA,” which is processed to yield crRNAs containing spacer-repeat fragments that guide effector nuclease complexes to cleave dsDNA sequences complementary to the spacer. Three different types of CRISPR systems are known, type I, type II, or type III, and classified based on the Cas protein type and the use of a proto-spacer-adjacent motif (PAM) for selection of proto-spacers in invading DNA. The endogenous type II systems comprise the Cas9 protein and two noncoding crRNAs: trans-activating crRNA (tracrRNA) and a precursor crRNA (pre-crRNA) array containing nuclease guide sequences (also referred to as “spacers”) interspaced by identical direct repeats (DRs). tracrRNA is important for processing the pre-crRNA and formation of the Cas9 complex. First, tracrRNAs hybridize to repeat regions of the pre-crRNA. Second, endogenous RNaseIII cleaves the hybridized crRNA-tracrRNAs, and a second event removes the 5′ end of each spacer, yielding mature crRNAs that remain associated with both the tracrRNA and Cas9. Third, each mature complex locates a target double stranded DNA (dsDNA) sequence and cleaves both strands using the nuclease activity of Cas9.

CRISPR/Cas gene editing systems have been developed to enable targeted modifications to a specific gene of interest in eukaryotic cells. CRISPR/Cas gene editing systems are commonly based on the RNA-guided Cas9 nuclease from the type II prokaryotic clustered regularly interspaced short palindromic repeats (CRISPR) adaptive immune system. Engineering CRISPR/Cas systems for use in eukaryotic cells typically involves reconstitution of the crRNA-tracrRNA-Cas9 complex. In human cells, for example, the Cas9 amino acid sequence may be codon-optimized and modified to include an appropriate nuclear localization signal, and the crRNA and tracrRNA sequences may be expressed individually or as a single chimeric molecule via an RNA polymerase II promoter. Typically, the crRNA and tracrRNA sequences are expressed as a chimera and are referred to collectively as “guide RNA” (gRNA) or single guide RNA (sgRNA). Thus, the terms “guide RNA,” “single guide RNA,” and “synthetic guide RNA,” are used interchangeably herein and refer to a nucleic acid sequence comprising a tracrRNA and a pre-crRNA array containing a guide sequence. The terms “guide RNA,” “guide sequence,” “guide,” and “spacer,” are used interchangeably herein and refer to the about 20 nucleotide sequence within a guide RNA that specifies the target site. In CRISPR/Cas9 systems, the guide RNA contains an approximate 20-nucleotide guide sequence followed by a protospacer adjacent motif (PAM) that directs Cas9 via Watson-Crick base pairing to a target sequence.

Cas protein families are described in further detail in, e.g., Haft et al., PLoS Comput. Biol., 1(6): e60 (2005), incorporated herein by reference. In one embodiment, the Cas9 protein is a wild-type Cas9 protein. The Cas9 protein can be obtained from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In some embodiments, the Cas9 protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas endonuclease is selected from Cas9, Cas12a, and Cas14.

In some embodiments, the Cas endonuclease employed is a Class 2 Cas endonuclease. In some embodiments, the Cas endonuclease is a Type V Cas endonuclease.

The compositions, systems, methods and uses thereof of the present invention are not limited only to CRISPR/Cas9 nuclease or other CRISPR/Cas nucleases for gene editing, but rather comprise and/or apply and can be used with or for use with any other gene editing DNA endonuclease that cleaves dsDNA such as but not limited to Talens or Zinc Finger Nucleases.

In some embodiments, the system comprises a nucleic acid molecule comprising a guide RNA sequence complementary to a target DNA sequence. The guide RNA sequence, as described above, specifies the target site with an approximate 20-nucleotide guide sequence followed by a protospacer adjacent motif (PAM) that directs Cas9 via Watson-Crick base pairing to a target sequence.

The terms “target DNA sequence,” “target nucleic acid,” “target sequence,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a Cas9/CRISPR complex, provided sufficient conditions for binding exist. In some embodiments, the target sequence is a genomic DNA sequence. The term “genomic,” as used herein, refers to a nucleic acid sequence (e.g., a gene or locus) that is located on a chromosome in a cell. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand.”

The target genomic DNA sequence may encode a gene product. The term “gene product,” as used herein, refers to any biochemical product resulting from expression of a gene.

Gene products may be RNA or protein. RNA gene products include non-coding RNA, including long non-coding RNA, such as XIST, such as tRNA, rRNA, micro RNA (miRNA), and coding RNA, such as messenger RNA (mRNA). In some embodiments, the target genomic DNA sequence encodes a protein or polypeptide.

In certain embodiments, provided herein are ubiquitin variants (that inhibit 53BPI) comprising variants of SEQ ID NO:1, with an amino acid modification at one or more of the following positions: 2, 4, 6, 8, 10, 11, 12, 14, 44, 46, 47, 48, 49, 62, 63, 64, 66, 68, 69, 70, 71, 72, 73, 74, 75, and 76. SEQ ID NO:1 has the following amino acid sequence:

(SEQ ID NO: 1)
Met Gln Ile Phe Val Lys Thr Leu Thr Gly Lys Thr
Ile Thr Leu Glu Val Glu Pro Ser Asp
Thr Ile Glu Asn Val Lys Ala Lys Ile Gln Asp Lys
Glu Gly Ile Pro Pro Asp Gln Gln Arg
Leu Ile Phe Ala Gly Lys Gln Leu Glu Asp Gly Arg
Thr Leu Ser Asp Tyr Asn Ile Gln Lys
Glu Ser Thr Leu His Leu Val Leu Arg Leu Arg Gly
Gly.

In certain embodiments, the amino acid modification in SEQ ID NO:1 is one or more of the following amino acids and positions: Position 2 is Leu or Arg, Position 4 is Tyr or Ile, Position 6 is Thr, Position 8 is Phe or Asp, Position 9 is Ala or Met, Position 10 is Arg or Trp, Position 11 is Met, Position 12 is Pro or Arg, Position 14 is Ser, Position 44 is Ala or Tyr, Position 46 is Gly, Position 47 is Glu, Asp or Ala, Position 48 is Met or Ser, Position 49 is Arg, Asp or Ser, Position 62 is Lys or Leu, Position 63 is Asn, Position 64 is Asp, Position 66 is Ser or Lys, Position 67 is Leu or Lys, Position 68 is Phe, Asn, or Leu, Position 69 is Pro, Position 70 is Leu, Position 71 is Val, Position 72 is Lys or Asn, Position 73 is Asn, Position 74 is Ser or Leu, Position 75 is Val or Arg or is absent, and Position 76 is Thr or Val or is absent. In some preferred embodiments at least one mutation is present in every gene in every domain and every subdomain that has a function encoded by said nucleic acid. Additional combinations of positions and amino acid changes are shown in U.S. Pat. Pub. 20190010196, which is herein incorporated by reference, and specifically for ubiquitin variants that can inhibit 53BPI. In certain embodiments, the ubiquitin variant comprises SEQ ID NO:2:

(also known as i53)
Met-Leu-Ile-Phe-Val-Lys-Thr-Leu-Thr-Gly-Lys-Thr-
Ile-Thr-Leu-Glu-Val-Glu-Pro-Ser-
Asp-Thr-Ile-Glu-Asn-Val-Lys-Ala-Lys-Ile-Gln-Asp-
Lys-Glu-Gly-Ile-Pro-Pro-Asp-Gln-
Gln-Arg-Leu-Ala-Phe-Ala-Gly-Lys-Ser-Leu-Glu-Asp-
Gly-Arg-Thr-Leu-Ser-Asp-Tyr-
Asn-Ile-Leu-Lys-Asp-Ser-Lys-Leu-His-Pro-Leu-Leu-
Arg-Leu-Arg.

In certain embodiments, the ubiquitin variant comprises an amino acid sequences with at least 55 . . . 60 . . . or 65 consecutive amino acids from SEQ ID NO:2. In other embodiments, the ubiquitin variant comprises at least 85% . . . 90% . . . 95% . . . 98 or 99% sequence identity with SEQ ID NO:2.

### I. Obtaining Granulocytes, NK Cells, and Lymphocytes

Granulocytes, NK cells, and/or lymphocytes can be harvested from blood. In some examples, the granulocytes, NK cells, and/or lymphocytes are obtained from the same subject to be treated (autologous, i.e., the donor and recipient are the same person or subject). The obtained autologous granulocytes, autologous NK cells, and/or autologous lymphocytes (which may be purified or isolated, or not, for example may be simply present in an apheresis product (e.g., leukapheresis product)), are treated with the compositions herein to correct a defective or protein, thereby generating recombinant autologous granulocytes, recombinant autologous NK cells, and/or recombinant autologous lymphocytes. The population of granulocytes, NK cells, and/or lymphocytes used to generate the recombinant autologous granulocytes, recombinant autologous NK cells, and/or recombinant autologous lymphocytes, and the recombinant autologous granulocytes, recombinant autologous NK cells, and/or recombinant autologous lymphocytes administered to a subject, do not need to be 100% pure; lower amounts of purity are acceptable. For example, a population of cells that contains at least 50%, at least 60%, at least 70%, at least 80%, at least 90%, at least 95%, or at least 99% autologous granulocytes, autologous NK cells, and/or autologous lymphocytes, or recombinant autologous granulocytes and/or lymphocytes, can be used. In some examples, unpurified autologous granulocytes, autologous NK cells, and/or autologous lymphocytes are used, such as by directly using an apheresis (e.g., leukapheresis) product obtained, without isolating the autologous granulocytes, autologous NK cells, and/or autologous lymphocytes from the apheresis/leukapheresis product, prior to introducing the compositions herein into such cells.

### A. Peripheral Blood/Apheresis

To obtain granulocytes, NK cells, and/or lymphocytes from the circulating peripheral blood, subjects can be injected with a cytokine, such as granulocyte colony-stimulating factor (G-CSF) (e.g., filgrastim, Neupogen, Amgen), to induce cells to leave the bone marrow and circulate in the blood vessels. Side effects of G-CSF, including headache, bone pain, and myalgia, can be treated with acetaminophen or narcotics. For example, the subject can be injected with G-CSF before the cell harvest. In one example, G-CSF (e.g., at least 5 mcg/kg/day, such as at least 10 mcg/kg/day, or at least 12 mcg/kg/day, such as about 5, 6, 7, 8, 9, 10, 11, 12, 13, 14, 15 or 16 mcg/kg/day) is administered subcutaneously to subjects daily for at least five days (such as 5, 6, 7, 8, 9 or 10 days). Doses can be given early in the morning, such as at least one hour and in some examples two hours prior to starting apheresis. G-CSF can be administered according to a vial-based algorithm to reduce wastage and increase the G-CSF dose given to lighter weight donors to improve yields as shown below. A mobilized peripheral blood stem cell (PBSC) concentrate can then be collected by leukapheresis, for example about 2 hours after the last dose of G-CSF.

Donors can receive prophylactic continuous intravenous calcium chloride infusions to prevent citrate toxicity during apheresis. The volume processed per apheresis procedure can be determined by medical staff on the day of apheresis, based on peripheral blood white cell increase in response to G-CSF, optimum and minimum cell dose needed, and kilogram weight of recipient. Volume of blood processed can range from 12 to 30 liters per procedure for 1 to 2 consecutive daily procedures, not to exceed a total of 60 liters over 2 days. In pediatric subjects, defined as less than 40 kg, a maximum of 8 blood volumes can be processed per procedure, for up to 2 consecutive daily procedures. In children less than 18 kg undergoing autologous leukapheresis procedures, three additional considerations apply. A central venous double-lumen catheter can be used for apheresis. It may be necessary to “prime” the apheresis instrument with a unit of allogeneic red cells, due to the fact that the volume of blood in the device during apheresis will exceed the safe extracorporeal volume (SEV) allowed for the patient. The SEV is generally about 15% of circulating blood volume or 10.5 mL/kg. In extremely small children (less than 16 kg), it may not be possible to use citrate anticoagulant without risk of severe citrate toxicity, thus, systemic heparinization can be used during apheresis. In some examples, subjects undergoing apheresis also receive calcium infusions (such as about 2 mg/mL). In some examples, subjects undergoing apheresis also receive one dose of pleraxifor, for example if stem cells are also being collected. The resulting apheresis sample can be used directly for the contacting with the compositions herein, or granulocytes and/or lymphocytes can be isolated from the apheresis product prior to contact with the compositions herein.

### B. Isolation Methods

Once cells are obtained from the blood, the granulocytes, NK cells, and/or lymphocytes in the sample are optionally isolated or purified. However, in some examples the material obtained from the apheresis is used directly. Any methods of separating or isolating the granulocytes, NK cells, and/or lymphocytes from such samples can be used. Negative and positive selection methods can be used. Negative selection methods take advantage of cell surface markers which are not expressed on granulocytes, NK cells and/or lymphocytes. Positive selection methods take advantage of cell surface markers, such as CD34 and CD133 that are expressed on granulocytes, NK cells, and/or lymphocytes. In one example, hydroxyethyl starch (HES) is not used.

In one example, methods are used that deplete non-granulocytes and/or non-lymphocytes from the sample, thereby permitting enrichment of the granulocytes and/or lymphocytes (that is, negative selection). For example, methods that substantially reduce the number of B cells, T cells, NK cells, dendritic cells, monocytes, and/or red blood cells can be used. In one example, labeled antibodies specific for the undesired cells can be incubated with the sample, allowing the labeled antibodies to bind to the undesired cells. Separation methods can then be used to remove those cells from the sample. For example, if the antibody label (such as biotin) is mixed with ferromagnetic particles coated with streptavidin, then passing the mixture through columns in the presence of a magnetic field can be used to remove the undesired cells. Thus, after incubation with the labeled antibodies, the sample is applied to the column, such that undesired cells bind to the column, while the granulocytes and/or lymphocytes pass through the column and can be collected. In some examples, the label is a fluorophore and flow cytometry can be used to remove the cells. In some examples, methods are used to deplete RBCs, for example by incubating the apheresis product with ACK lysis buffer to lyse RBCs in the apheresis product. Similarly, methods can be used to deplete non-NK cells, thereby enriching for NK cells. In addition, commercially available kits can be used to deplete non-granulocytes, non-NK cells, and/or non-lymphocytes from the sample, such as those from Miltenyi.

In one example, methods are used that recover granulocytes, NK cells, and/or lymphocytes from the sample by elutriation, thereby permitting enrichment of the granulocytes and/or lymphocytes (positive selection). In one example, labeled antibodies specific for granulocytes, NK cells, and/or lymphocytes can be incubated with the sample, allowing the labeled antibodies to bind to the granulocytes, NK cells, and/or lymphocytes, and subsequent recovery of the labeled granulocytes, NK cells, and/or lymphocytes. In one example, the sample is exposed or incubated with labeled Miltenyi antibodies, thereby labeling the granulocytes, NK cells, and/or lymphocytes. The labeled granulocytes, NK cells, and/or lymphocytes can then be recovered, for example using flow cytometry (e.g., if the label is a fluorophore) or by use of a column (e.g., if the label is a magnetic label, such as magnetic beads containing CD3 for T cells). The resulting granulocytes, NK cells, and/or lymphocytes can be used immediately to generate recombinant granulocytes, NK cells, and/or lymphocytes, or frozen for future use (for example frozen in growth media containing DMSO).

C. Culturing Granulocytes and/or Lymphocytes

The blood or apheresis product, or the isolated granulocytes, NK cells, and/or lymphocytes, can be cultured ex vivo, for example to expand the blood, apheresis product, granulocytes, NK cells, and/or lymphocytes, prior to introducing an exogenous mRNA into the cells. In some examples the cells (such as lymphocytes and/or NK cells) are grown in RPMI, plus animal or human serum, such as fetal calf serum. In some examples the growth media further includes cytokines, amino acids, and other growth supplements. In some examples, the blood or apheresis product, or the isolated granulocytes, NK cells, and/or lymphocytes, are not cultured prior to transfection with the desired mRNA and other compositions herein. For example, the resulting cells (e.g., granulocytes) can be washed in a buffer, such as an electroporation buffer containing albumin, such as 0.1 to 5% HSA, such as about 1% HSA.

### II. Correcting Genetic Defects

Granulocytes, such as neutrophils, NK cells, and/or lymphocytes from the subject to be treated, are made recombinant by introducing the compositions herein (e.g., TP53 inhibitor, 53BPI inhibitor, and CRISPR/Cas components) to correct the defective protein in the subject. The resulting recombinant granulocytes, recombinant NK cells, and/or recombinant lymphocytes, are introduced into the subject.

Although not necessary, detectable markers (e.g., fluorescent protein, such as GFP) or selection markers (e.g., antibiotic resistance) can be introduced along with the compositions herein, to permit the identification of cells with the expressing the corrected protein. In some examples introduced marker(s) is removed prior to introduction into the subject. The autologous granulocytes, autologous NK cells, and/or autologous lymphocytes used can be previously cultured ex vivo (for example to increase their numbers), used directly following isolation from a blood or apheresis product, or used directly from a blood or an apheresis product (e.g., unpurified).

In some examples, the a donor oligonucleotide is used with the Crispr/Cas system to correct the defective gene in a CDG patient, where the defective gene encodes a native or wild-type CYBA (p22phox), CYBB (gp91phox), NCF1 (p47phox), NCF2 (p67phox), NCF4 (p40phox), or combinations thereof.

In some examples, the corrected gene encodes a protein that is missing or defective in a XMEN patient, such as encodes a native or wild-type MagT1 protein. In some examples, the corrected gene encodes a protein that is defective in a CTLA4 deficient patient, such as encodes a native or wild-type CTLA4 protein.

The mRNAs and donor oligonucleotides herein can have, for example, their Ts replaced with uridines, or an unnatural nucleoside such as pseudouridine. Such sequences introduced in the disclosed autologous granulocytes, autologous NK cells, and/or autologous lymphocytes (generating recombinant cells) and used in the disclosed methods can further include (1) a 5-end cap, (2) a 3-end poly-A tail (such as 150 or more As), (3) a 3-UTR (such as a human or Xenopus beta globin 3-UTR, (4) a 5-UTR (such as a human or Xenopus beta globin 5′-UTR, (which can include a Kozak sequence), (5) can be codon optimized for expression in a human cell, and/or (6) include one or more pseudouridines or other unnatural nucleoside in place of one or more uridines (or Ts) (e.g., replace at least 90%, at least 95%, at least 99%, or 100% of all U or Ts).

### III. In Vitro-Transcribed RNA (IVT-RNA)

In certain embodiments sgRNAs are manufactured chemically (e.g., on an oligonucleotide synthesizer) (e.g., by a commercial vendor that has the capability to synthesize oligoribodeoxynucleotides) that comprise at least three ribonucleotides with phosphorothioated bonds or another modified phosphoryl group that is resistant to exonucleases on each end of sgRNAs. In certain embodiments, the sgRNAs, and the mRNAs encoding the Cas 9 or other Cas proteins or other gene-editing enzymes, the TP53 inhibitor proteins, and/or the 53BPI inhibitor proteins that were used for or in any of the compositions, kits, systems, methods, or uses described in the present application were made by in vitro transcription (IVT) of a DNA template that encodes said respective sgRNAs, Cas proteins or other gene-editing enzymes, proteins that are TP53 inhibitors, and/or proteins that are 53BPI inhibitors. In some embodiments, the DNA template used for IVT may be digested using recombinant animal-origin free deoxyribonuclease preparation. Then, the IVT-RNA can be purified by organic extraction and/or by ammonium acetate precipitation, which selectively precipitates RNA, leaving most of the proteins, DNA digestion products, unincorporated NTPs and other reaction mixture components in the supernatant. Precipitation of the RNA with 70% ethanol, may also be employed to remove salt.

The purified IVT-RNA can then be polyadenylated enzymatically, for example using A-Plus™ PolyA Polymerase as described by the manufacturer (CELLSCRIPT, Madison, Wis., USA) or another commercially available polyA polymerase as described by said manufacturer. Polyadenylated IVT-RNA may be further purified as described for the IVT-RNA. In some embodiments, a polyA tail is added to the IVT-RNA co-transcriptionally by using a DNA template that encodes the tail. The polyadenylated IVT-RNA can be capped enzymatically capped post-transcriptionally, using a capping enzyme, such asScriptCap™ Capping Enzyme, to generate 5′-capped and 3′-polyadenylated IVT-RNA having a cap with a cap0 structure, or, if desired, the polyadenylated IVT-RNA is enzymatically capped using both ScriptCap™ Capping Enzyme and ScriptCap™ 2′-O-Methyltransferase to generate 5′-capped and 3′-polyadenylated IVT-RNA having a cap with a cap1 structure as described by the manufacturer (CELLSCRIPT, Madison, Wis., USA). Alternatively, a 5′ cap can be added to the IVT-RNA co-transcriptionally by using a cap analog, such as an anti-reverse cap analog (ARCA), by primer extension of a capped oligoribonucleotide annealed to the linearized DNA template by the RNA polymerase, or using CleanCap™ technology as described by the manufacturer Cap (TriLink™ Biotechnologies).

Additional details of the above materials and processes and other information, such as chemical structures for 5′ caps with cap0 and cap1 structures are available in the online pdf files on the Products page at www.cellscript.com. In some embodiments, the purified IVT-RNA is capped using one of the above methods prior to polyadenylations with polyA polymerase as described above. In some other embodiments, the IVT is performed using a linear DNA template that comprises a polydT) sequence for synthesis of the IVT-RNA with a 3′ polyA tail. Preferably the oligo(dT) sequence does not comprise more than about 50 Ts, in order to maintain the stability of the plasmid template. In some embodiments the polyA tail that was template-encoded is further extended post-transcriptionally using a polyA polymerase.

If the 5-Capped and polyadenylated IVT-RNA (mRNA) is for use in making a therapeutic product, including for making a cell therapy by introducing the mRNA into a human or non-human animal cell (e.g., a mammalian cell) or is for use in therapy, it is important that the mRNA, in some embodiments, is further purified to remove RNA purities that would be immunogenic and toxic by inducing an innate immune upon such introduction into a human or non-human animal cell (e.g., a mammalian cell) or into a human or non-human animal (e.g., mammalian) body. In addition to the above processes, RNA (including IVT-RNA, polyadenylated RNA, 5′-capped and polyadenylated IVT-RNA or mRNA) for therapeutic or other uses in or on humans or animals may be further purified using other purification processes, such as using processes that include HPLC or gravity flow column purification of mRNA and/or treatment of said RNA with RNase III, followed by clean-up steps to remove the enzyme and other enzyme digestion products (e.g., as disclosed in U.S. Pat. No. 10,201,620, herein incorporated by reference). In some embodiments, mRNA or IVT-RNA comprising a poly-A tail is purified using a POROS™ Oligo(dT)25 affinity resin. (Thermo Fisher™ Scientific) or using a CIMmultus™ Oligo-dT column (BIA Separations) according to the instructions of those respective manufacturers.

In some examples, RNA for therapeutic or other uses involving administration to humans or animals or cells therefrom, are purified such that said RNA is free of double-stranded RNA (dsRNA) having a length of greater than about 40 basepairs (bps) in length. For example, in some cases, the amount of dsRNA in said purified RNA for therapeutic or other use in humans or animals (for example present in a therapeutic composition) is quantified using an immunofluorescent assay to detect the amount of a dsRNA-specific mAb that binds to dsRNA that is present in RNA spotted on nylon membrane dot blots on which controls containing known amounts of the tested RNA or dsRNA are also spotted as controls. For example, in some cases using this assay, the amount of dsRNA contaminant molecules that have a size of greater than 40 basepairs (bp) in length are quantified using a dsRNA-specific J2 mAb or K1 mAb (e.g., from English & Scientific Consulting, Kft., Szirák, Hungary (also known as SCICONS)), which mAbs have the IgG isotype (igG2a subclass) and only efficiently bind dsRNA longer than 40 bp in length, in a sequence-independent manner.

In some cases, the amount of dsRNA contaminant molecules having a size of greater than 40 bp in said RNA following purification of RNA for therapeutic or other use in humans or animals following purification is less than 0.1% of the total mass (i.e., weight) of the RNA. In some cases, the amount of dsRNA contaminant molecules having a size of greater than 40 bp in said RNA following purification of the RNA is less than 0.01% of the total mass (i.e., weight) of the RNA. In some cases, the amount of dsRNA RNA contaminant molecules that have a size of greater than 40 bp in said RNA following purification is less than 0.001% of the total mass (i.e., weight) of the RNA.

For example, in some cases, said RNA (e.g., 25-100 ng) can be blotted onto a nitrocellulose membrane, allowed to dry, blocked with 5% non-fat dried milk in TBS buffer supplemented with 0.05% Tween-20 (TBS-T), and incubated with a dsRNA-specific J2 mAb or K1 mAb for 60 minutes. Membranes are washed six times with TBS-T and then reacted with HRP-conjugated donkey anti-mouse antibody (Jackson Immunology). After washing six times, dsRNA can be detected with the addition of SuperSignal West Pico Chemiluminescent substrate (Pierce) and image capture for 30 seconds to two minutes on a Fujifilm LAS1000 digital imaging system. In still other immunofluorescent assays of the amount of dsRNA in purified RNA using the J2 or K1 mAbs can be carried out using a second Ab that has different label and the amount of dsRNA in said RNA is quantified using a digital bioimager that can detect said different label.

In certain embodiments, the RNAs and protein-encoding mRNAs (or mRNA molecules) of the compositions, systems, methods, uses and other embodiments of the present invention are purified to remove RNA contaminants that are toxic and immunogenic by inducing an innate immune response. In vitro methods and assays for determining the relative immunogenicity of different substances are well known in the art, and include, e.g. measuring the quantity of a cytokine (e.g., a cytokine selected from the group consisting of IL-12, IFN-alpha, TNF-alpha, RANTES, MIP-1-alpha, MIP-1-beta, IL-6, IFN-beta, and IL-8), that is secreted during a defined period of time after each sample comprising a defined number of one type of cells (e.g., human monocyte-derived dendritic cells (MDDCs)) has been transfected with one of the substances (e.g. 1, a substance comprising a given amount of an mRNA prior to purification, e.g., 2, a substance comprising the same amount of the mRNA after purification), and then comparing the quantity of the respective cytokine secreted measured in response to each substance.

Those with knowledge in the art will understand that the in vitro methods and assays described above are for determining the relative immunogenicity of different substances based on quantifying one or more innate immune responses (e.g., secretion of a cytokine) that occurs immediately in response to a cell's detection of a substance (rather than to methods or assays that involve quantifying an adaptive immune response).

In some embodiments, the relative immunogenicity of the purified mRNA compared to the purified mRNA is determined by measuring the quantity of a cytokine selected from IL-12, IFN-alpha, and TNF-alpha secreted during a defined time period after transfection of a sample comprising a defined number of MDDCs with the purified mRNA compared to the quantity of said cytokine secreted during said defined time period after transfection of said sample comprising said defined number of MDDCs with the unpurified mRNA, wherein the amount of said cytokine secreted in response to transfection with the purified mRNA is significantly less than the amount of said cytokine secreted in response to transfection with the unpurified mRNA.

In some embodiments, the relative immunogenicity of two substances is expressed as a fold difference based on the relative quantities of said cytokine secreted. For example, if one-half as much cytokine is secreted from a first sample comprising said defined number of MDDCs transfected with a first substance (e.g. the purified mRNA) than is secreted from a second sample comprising said defined number of MDDCs transfected with a second substance (e.g. the unpurified mRNA), then the first substance (e.g. the purified mRNA) is said to be two-fold less immunogenic than the second substance (e.g. the unpurified mRNA). In other embodiments the fold decrease in immunogenicity is 2-5-fold, 5-10-fold, 10-20-fold, or any fold-decrease that is empirically determined. In some embodiments, the fold decrease that is determined is statistically different from a result that is found for a different substance or a different sample having a different composition, or that is purified using a different method, or that is subjected to some other condition for a particular reason. In some embodiments, the immunogenicity of the substance is not greater than a negative control that does not comprise the substance (e.g., the RNA), meaning that the immunogenicity is not greater than the background level that lacks the substance. In another embodiment, background levels of secretion in the absence of any substance (a mock control) are subtracted before calculating the relative fold immunogenicity in the above methods. Each possibility represents a separate embodiment of the present invention.

### Human MDDCs

MDDCs can be purchased from commercial sources or prepared from human monocytes. For preparation of cytokine-generated MDDC, monocytes are purified from PBMC by discontinuous Percoll gradient centrifugation. The low density fraction (monocyte enriched) is depleted of B, T, and, NK cells using magnetic beads (Dynal, Lake Success, N.Y.) specific for CD2, CD16, CD19, and CD56, yielding highly purified monocytes as determined by flow cytometry using anti-CD14 (>95%) or antiCD11c (>98%) mAb. To generate immature DC, purified monocytes are cultured in AIM V serum-free medium (Life Technologies), supplemented with GM-CSF (50 ng/ml)+IL-4 (100 ng/ml) (R & D Systems, Minneapolis, Minn.) in AIM V medium (Invitrogen) for the generation of monocyte-derived DC (MDDC) as described (Weissman, D et al, 2000. J Immunol 165: 4710-4717). MDDC also can be generated by treatment with GM-CSF (50 ng/ml)+IFN-alpha (1,000 V/ml) (R & D Systems) to obtain IFN-alpha MDDC (Santini et al., 2000. Type I interferon as a powerful adjuvant for monocyte-derived dendritic cell development and activity in vitro and in Hu-PBL-SCID mice. J Exp Med 191: 1777-178).

### MDDCs Immunogenicity Assays

MDDCs Immunogenicity assays can be performed in 96-well plates (approximately 10⁵ cells/well) are treated with R-848, Lipofectin™, or Lipofectin™-RNA for 1 h, then the medium is changed.

At the end of 8 h (unless otherwise indicated), cells are harvested for either RNA isolation or flow cytometry, while the collected culture medium is subjected to cytokine ELISA. The levels of IL-12 (p70) (BD Biosciences Pharmingen, San Diego, Calif.), IFN-alpha, TNF-alpha, and IL-8 (Biosource International, Camarillo, CA) are measured in supernatants by sandwich ELISA. Cultures are performed in duplicate, triplicate or quadruplicate. Levels of TNF-alpha, INF-alpha or IL-12 can also be assayed by immunoassays on Western blots. In some other embodiments, relative immunogenicity of purified mRNAs compared to unpurified mRNAs are determined by quantifying levels of mRNAs encoding TNF-alpha, INF-alpha or IL-12 in MDDCs by RT-PCR at different time points after separately transfecting a defined number of said MDDCs with the same amount of the purified and unpurified mRNA, respectively and then determining the difference in the level of said cytokine mRNA in said MDDCs transfected with the purified mRNA compared to the level of said cytokine mRNA in said MDDCs transfected with the unpurified mRNA, and then comparing those levels to determine the relative immunogenicity.

In some examples, the mRNA used in the disclosed methods includes a cap on its 5′-end. The cap on the 5′-end of the mRNA can include a guanine nucleoside that is joined via its 5-carbon to a triphosphate group that is, in turn, joined to the 5′-carbon of the most 5′-nucleotide of the IVT-RNA. In some examples, the 5′-end of the mRNA is enzymatically capped with a capping enzyme with RNA triphosphatase, RNA guanyltransferase and guanine-7-methyltransferase activities, which yields an N7-methylguanosine standard cap with a cap0 structure. In some other cases, the mRNA with an N7-methylguanosine standard cap is further modified using 2′-O-methyltransferase, which 2′-O-methylates the 2′ position of the 5′-penultimate nucleotide with respect to the N7-methylguanosine standard cap, thereby generating a dinucleotide cap on the RNA which has a cap1 structure. An anti-reverse cap analog (ARCA), which has a structure of m2 7,3-O G(5′)ppp(5′)G; P-(5′-(3′-O-methyl)-7-methyl-guanosyl) P3-(5′-(guanosyl))triphosphate), can be used for transcription during the IVT. However, about 20% of the IVT-RNA may not be capped and will not be translated because the cap analog is usually present in a 4/1 molar ratio GTP during the IVT reaction. Other exemplary 5′-end caps (and methods on how to incorporate them onto a nucleic acid molecule) are provided in US Application Publication No. 20140221248 (herein incorporated by reference). In certain embodiments, CLEANCAP (TriLink Biotechnologies) is employed for capping.

In some examples, the mRNA used in the disclosed methods includes a poly-A tail at its 3′-end. In some examples, the poly-A tail is at least 150 adenosines (As), at least 200 As, at least 250 As, at least 300 As, at least 400 As, or at least 500 As, such as about 150, 160, 170, 180, 190, 200, 250, 300, 350, 400, 450, or 500 As. In some embodiments, the number of adenosines in the polyA tail is an amount ≥50, ≥100, ≥150, ≥175, ≥200, ≥250, or ≥300.

In many embodiments the mRNAs comprising the compositions, systems, or kits or used in the methods or uses of the present invention comprise one or more modified nucleic acid bases, nucleoside or nucleotides or ribonucleotides. In some embodiments, the mRNA comprises a modified nucleoside selected from pseudouridine (Ψ), 1-methylpseudouridine (m¹Ψ), 5-methyluridine (m⁵U), 5-methoxyuridine (mo⁵U) and 2-thiouridine (s²U) in place of uridine, which mRNAs are produced by in vitro transcription (IVT) of a DNA template with an RNA polymerase (e.g., a phage T7, T3 or SP6 RNA polymerase) in a reaction mixture wherein the uridine-5′-triphosphate (UTP) is replaced by the respective ΨTP, m¹ΨTP, m⁵UTP, mo⁵UTP, or s²UTP in the desired proportion to which the user wants the uridines to be replaced by the modified nucleotide. In some embodiments, mRNAs comprising Ψ, m¹Ψ, m⁵U, mo⁵U or s²U in place of U are more stable than their counterpart mRNAs comprising only U. One major benefit of mRNAs comprising Ψ, m¹Ψ, m⁵U, mo⁵U or s²U in place of U is that they are less immunogenic when introduced into mammalian or other cells than the counterpart mRNAs do not comprise Ψ, m¹Ψ, m⁵U, mo⁵U or s²U in place of U. In most embodiments wherein Ψ, m¹Ψ, m⁵U, or mo⁵U is the modified nucleoside used, all of the uridines are replaced by the respective Ψ, m¹Ψ, m⁵U, or mo⁵U in the mRNA because mRNAs with all of the uridines replaced by one of those modified nucleosides are usually expressed into protein as well or, often, at higher levels than their counterpart mRNAs comprising only uridines. However, since mRNA comprising 2-thiouridine (s²U) in place of all of the canonical uridines is not expressed well, mRNA comprising s²U is usually produced with a lower proportion of the uridines replaced, e.g., with about 5-70%, 10-15%. 15-20%, 20-30%, 30-40%, 40-50%, 50-60%, 60-70%, or less than 50%, less than 40%, less than 30% or less than 20% of the uridines replaced by s²U. Commonly, about 10%, about 15%, about 20% or about 25% of the uridines replaced by s²U. In some embodiments, wherein an mRNA comprises some or all of their uridines replaced by Ψ, m¹Ψ, m⁵U, mo⁵U or s²U, the mRNA s²U, the mRNA further comprises 5-methylcytidine (m⁵C) in place of some or all of the canonical cytidine nucleotides, which is produced by also replacing any desired proportion (up to 100%) of the canonical CTP by m⁵CTP in the in vitro transcription reaction.

The reduced immunogenicity of the mRNAs comprising Ψ, m¹Ψ, m⁵U, mo⁵U or s²U relative to the immunogenicity of mRNA not comprising Ψ, m¹Ψ, m⁵U, mo⁵U or s²U in place of U can be determine using the same well known in the art in vitro methods and assays for determining the relative immunogenicity of mRNAs, with different levels of purity with respect to contaminants (e.g., RNA contaminants) that are immunogenic by inducing an innate immune response comprising secretion of pro-inflammatory or inflammatory cytokines. As discussed previously, such methods include, e.g. measuring the quantity of a cytokine (e.g., a cytokine selected from the group consisting of IL-12, IFN-alpha, TNF-alpha, RANTES, MIP-1-alpha, MIP-1-beta, IL-6, IFN-beta, and IL-8), that is secreted during a defined period of time after each sample comprising a defined number of one type of cells (e.g., human monocyte-derived dendritic cells (MDDCs)) has been transfected with one of the substances (e.g. 1, a substance comprising a given amount of an mRNA prior to purification, e.g., 2, a substance comprising the same amount of the mRNA after purification), and then comparing the quantity of the respective cytokine secreted measured in response to each substance.

In some embodiments, the relative immunogenicity of the mRNA comprising Ψ, m¹Ψ, m⁵U, mo⁵U or s²U in place of U compared to the mRNA not comprising Ψ, m¹Ψ, m⁵U, mo⁵U or s²U in place of U is determined by measuring the quantity of a cytokine selected from IL-12, IFN-alpha, and TNF-alpha secreted during a defined time period after transfection of a sample comprising a defined number of MDDCs with the mRNA comprising Ψ, m¹Ψ, m⁵U, mo⁵U or s²U in place of U compared to the quantity of said cytokine secreted during said defined time period after transfection of said sample comprising said defined number of MDDCs with the mRNA not comprising Ψ, m¹Ψ, m⁵U, mo⁵U or s²U in place of U, wherein the amount of said cytokine secreted in response to transfection with the mRNA comprising Ψ, m¹Ψ, m⁵U, mo⁵U or s²U is significantly less than the amount of said cytokine secreted in response to transfection with the mRNA not comprising Ψ, m¹Ψ, m⁵U, mo⁵U or s²U.

In some embodiments, the relative immunogenicity of two substances is expressed as a fold difference based on the relative quantities of said cytokine secreted. For example, if one-half as much cytokine is secreted from a first sample comprising said defined number of MDDCs transfected with a first substance (e.g. the mRNA comprising Ψ, m¹Ψ, m⁵U, mo⁵U or s²U in place of U) than is secreted from a second sample comprising said defined number of MDDCs transfected with a second substance (e.g. the mRNA not comprising Ψ, m¹Ψ, m⁵U, mo⁵U or s²U in place of U), then the first substance (e.g. mRNA comprising Ψ, m¹Ψ, m⁵U, mo⁵U or s²U in place of U) is said to be two-fold less immunogenic than the second substance (e.g. the mRNA not comprising Ψ, m¹Ψ, m⁵U, mo⁵U or s²U in place of U). In another embodiment, background levels of secretion in the absence of any substance (a mock control) are subtracted before calculating the relative fold immunogenicity in the above methods. Each possibility represents a separate embodiment of the present invention. mRNA molecules often have regions of differing sequence located before the translation start codon and after the translation stop codons that are not translated. These regions, termed the five prime untranslated region (5′ UTR) and three prime untranslated region (3′ UTR), respectively, can affect mRNA stability, mRNA localization, and translational efficiency of the mRNA to which they are joined. Certain 5′ and 3′ UTRs, such as those for alpha and beta globins, improve mRNA stability and expression of mRNAs. Thus, in some examples, the mRNAs provided herein include a 5′ UTR and/or a 3′ UTR that results in greater mRNA stability and higher expression of the mRNA in the cells (e.g., an alpha globin or a beta globin 5′ UTR and/or 3′ UTR; e.g., a Xenopus or human alpha globin or a beta globin 5′ UTR and/or 3′ UTR, or, e.g., a tobacco etch virus (TEV) 5′ UTR). However, other 5′ and 3′ UTRs in the human genome and other genomes can be used. A naturally occurring mRNA of interest may include its own 5′ and/or 3′-UTR. The 3′-UTR sequence immediately follows the translation termination codon and appears before the poly-A tail.

IV. Introduction of mRNA into Immune Cells

mRNAs that encode TP53 inhibitors, 53BPI inhibitors, and Cas proteins, can be introduced into granulocytes, NK cells, and/or lymphocytes (such as an apheresis product), thereby generating recombinant granulocytes, recombinant NK cells, and/or recombinant lymphocytes. In one example, naked nucleic acid molecules are used.

Blood, apheresis product, leukapheresis produce, isolated granulocytes (such as neutrophils), isolated NK cells, and/or isolated lymphocytes, obtained from the subject can be used directly for transfection. However, in some examples such cells are incubated in a culturing medium in a culture apparatus for a period of time or until the cells reach sufficient number before passing the cells to another culture apparatus. The culturing apparatus can be of any culture apparatus commonly used for culturing cells in vitro. In one example, the level of confluency of the cells is greater than 70%, or greater than 90% before passing the cells to another culture apparatus. A period of time can be any time suitable for the culture of cells in vitro. The culturing medium may be replaced during the culture of the cells. The cells are then harvested from the culture apparatus. The cells can be used immediately or they can be cryopreserved (for example in the presence of DMSO) and stored for use at a later time.

Blood, apheresis product, leukapheresis produce, isolated granulocytes (such as neutrophils), isolated NK cells, and/or isolated lymphocytes, to be transfected can be grown in culture. Culture media typically contains a variety of essential components required for cell viability, including inorganic salts, carbohydrates, hormones, essential amino acids, vitamins, and the like. In some embodiments, RPMI is used as a culture medium. Additional additives can be used, such as glutamine, heparin, sodium bicarbonate, serum and/or N2 supplement. The pH of the culture medium is typically between 6-8, such as about 7, for example about 7.4. Cells can be cultured at a temperature between 30-40° C., such as between 35-38° C., such as between 35-37° C., for example at 37° C.

Methods for introducing nucleic acid molecules, which include the desired mRNAs herein (e.g., encoding TP53 inhibitors, 53BPI inhibitors, and Cas proteins) into granulocytes, NK cells, and/or lymphocytes (such as an apheresis product) in culture include chemical and physical methods. Chemical methods include liposome-based gene transfer or lipofection, lipid nanoparticles (LNPs), calcium phosphate-mediated gene transfer, DEAE-dextran transfection techniques, and polyethyleneimine (PEI)-mediated delivery. Physical methods include ballistic gene transfer (introduces particles coated with nucleic acid molecules into cells), microinjection, and nucleofection. In a specific example, granulocytes, NK cells, and/or lymphocytes (such as an apheresis product) are electroporated to allow entry of the mRNAs into the cells. For example, the electroporation can be performed using a GMP-compliant MaxCyte Biosystems (for example at room temperature). In some examples, at least 100 ug/ml of mRNA is used for the transfection, such as at least 200 ug/ml of mRNA, at least 300 ug/ml of mRNA, at least 400 ug/ml of mRNA, or at least 500 ug/ml of mRNA, such as 200 to 400 ug/ml of mRNA, for example with cells at a concentration of at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, or at least 7.5×10⁸ cells/ml, such 5-7.5×10⁸ cells/ml. In some examples, at least 50 million cells/ml are transfected, such as at least 100 million cells/ml, at least 200 million cells/ml, at least 500 million cells/ml or at least 750 million cells/ml. Following transfection, cells can be incubated at 37° C., for example cultured at 5-7×10⁶ cells/mL

V. Administration of Recombinant Immune Cells into a Subject

The recombinant autologous granulocytes (e.g., recombinant autologous neutrophils), recombinant autologous NK cells, and/or recombinant autologous lymphocytes, can be introduced, that is administered or transplanted, into a subject, such as a subject with a PID. In some examples, the subject with PID has a chronic infection. In some examples, the subject is treated prior to receiving a HSC or bone marrow transplant. In some examples, the subject with PID has autoimmune disease, such as Hashimoto's thyroiditis, pernicious anemia, inflammatory bowel disease (Crohn's disease and ulcerative colitis), psoriasis, renal, pulmonary, and hepatic fibroses, Addison's disease, type I diabetes, systemic lupus erythematosus, dermatomyositis, Sjogren's syndrome, multiple sclerosis, myasthenia gravis, Reiter's syndrome, rheumatoid arthritis, or Grave's disease. Thus, transplantation of the recombinant autologous granulocytes and/or recombinant autologous lymphocytes can be used to treat patients with PID, such as one with a chronic infection, acute infection, or autoimmunity.

In some examples therapeutically effective amounts include at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 2.5×10⁷, at least 5×10⁷, at least 1×10⁸, at least 2.5×10⁸, or at least 5×10⁸ of recombinant autologous granulocytes, recombinant autologous NK cells, and/or recombinant autologous lymphocytes. Such amounts can be introduced into the recipient subject, for example by injection, such as intravenously. In some examples, multiple separate therapeutic doses of recombinant autologous granulocytes, recombinant autologous NK cells, and/or recombinant autologous lymphocytes are administered to the subject. For example, the recombinant autologous granulocytes, recombinant autologous NK cells, and/or recombinant autologous lymphocytes can be administered to the subject at least twice, at least 5 times, at least 10 times, at least 20 times, at least 40 times, at least 50 times, at least 75 times, at least 100, or at least 500 different times. In some examples, the subject receives such recombinant cells over the course of their entire life.

### A. Subjects

In some examples, the subject receiving the recombinant granulocytes and/or recombinant lymphocytes (which in some examples are autologous) can have a PID, such as one disclosed herein. The subject can be a mammal, such as a human, or veterinary subject. In some examples the subject is a pediatric subject (e.g., less than one year old), child (e.g., less than 18 years old), or an adult (e.g., at least 18 years old).

In some examples, the subject has an autoimmune disease as a result of a PID, such as Hashimoto's thyroiditis, pernicious anemia, inflammatory bowel disease (Crohn's disease and ulcerative colitis), psoriasis, renal, pulmonary, and hepatic fibroses, Addison's disease, type I diabetes, systemic lupus erythematosus, dermatomyositis, Sjogren's syndrome, multiple sclerosis, myasthenia gravis, Reiter's syndrome, rheumatoid arthritis, or Grave's disease. Thus, some examples, such autoimmune diseases are treated with the disclosed methods.

### B. Infections

In some examples, the subject with PID has an acute or chronic infection, such as an acute or chronic bacterial, viral, fungal, or parasitic infection. Such infections can be treated with the disclosed methods. In some examples, the subject has a Gram-positive or Gram-negative bacterial infection, such as one or more of: Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melitensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeium and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (e.g., Klebsiella pneumoniae, Klebsiella granulomatis and Klebsiella oxytoca), Lactobacillus sp., Listeria sp. (e.g., Listeria monocytogenes), Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (e.g., Mycobacterium leprae, Mycobacterium tuberculosis, Mycobacterium intracellulare, Mycobacterium avium, Mycobacterium bovis, and Mycobacterium marinum), Mycoplasm sp. (e.g., Mycoplasma pneumoniae, Mycoplasma hominis, and Mycoplasma genitalium), Nocardia sp. (e.g., Nocardia asteroides, Nocardia cyriacigeorgica and Nocardia brasiliensis), Neisseria sp. (e.g., Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Pseudomonas cepacia, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (s e.g., s Salmonella enterica, Salmonella typhi, Salmonella paratyphi, Salmonella enteritidis, Salmonella cholerasuis and Salmonella typhimurium), Serratia sp. (e.g., Serratia marcesans and Serratia liquifaciens), Shigella sp. (e.g., Shigella dysenteriae, Shigella flexneri, Shigella boydii and Shigella sonnei), Staphylococcus sp. (e.g., Staphylococcus aureus, Staphylococcus epidermidis, Staphylococcus hemolyticus, Staphylococcus saprophyticus), Streptococcus sp. (such as Streptococcus pneumoniae (e.g., chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (e.g., Treponema carateum, Treponema petenue, Treponema pallidum and Treponema endemicum, Tropheryma whippelii, Ureaplasma urealyticum, Veillonella sp., Vibrio sp. (e.g., Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) or Xanthomonas maltophilia, among others.

In some examples, the subject has a positive-strand RNA viral infection, such as infection by one or more of a: Picornavirus (e.g., Aphthovirus [for example foot-and-mouth-disease virus (FMDV)], Cardiovirus; Enterovirus (e.g., Coxsackie viruses, Echoviruses, Enteroviruses, Rhinovirus and Polioviruses); Hepatovirus (e.g., Hepatitis A, B or C virus); Togavirus (e.g., rubella; alphaviruses (such as Western equine encephalitis virus, Eastern equine encephalitis virus, and Venezuelan equine encephalitis virus)); Flavivirus (e.g., Dengue virus, West Nile virus, and Japanese encephalitis virus); Calciviridae (e.g., Norovirus and Sapovirus); or Coronavirus (e.g., human coronavirus 229E, OC43, NL63, HKU1, SARS coronaviruses, and Middle East respiratory syndrome coronavirus).

In some examples, the subject has a negative-strand RNA viral infection, such as infection by one or more of an Orthomyxyovirus (e.g., influenza virus), Rhabdovirus (e.g., Rabies virus), or Paramyxovirus (e.g., measles virus, respiratory syncytial virus, and parainfluenza viruses). In some examples, the subject has a DNA viral infection, such as infection by one or more of a: Herpesvirus (e.g., Varicella-zoster virus, for example the Oka strain; cytomegalovirus; Herpes simplex virus (HSV) types 1 and 2, and Epstein-Barr virus), Adenoviruses (e.g., Adenovirus type 1 and Adenovirus type 41), Poxviruses (e.g., Vaccinia virus), and Parvoviruses (e.g., Parvovirus B19).

In some examples, the subject has a retroviral infection, such as infection by one or more of: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); or avian sarcoma virus. In some examples, the subject has an acute or chronic infection by Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, or Zika virus

In some examples, the subject has an infection with a protozoa, nemotode, or fungi.

Exemplary protozoa that may infect a subject treated herein include, but are not limited to, Plasmodium (e.g., Plasmodium falciparum: to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, and Toxoplasma. Exemplary fungi include, but are not limited to, Aspergillus sp. (including Aspergillus fumigatus), Candida sp., (such as Candida albicans), C. neoformans, C. gattii, Coccidioides sp., Coccidiodes immitis, Trichophyton sp., Microsporum sp., Epidermophyton sp., Tinea sp., and Blastomyces dermatitidis. In some examples, the subject has CGD and a Staphylococcus, Burkholderia, Nocardia, Serratia, Klebsiella, and/or Aspergillus infection.

### C. PIDs

PIDs that can be treated with the disclosed methods include any PID resulting from a genetic defect. In some examples, the PID is caused by a loss of function, that is, the protein is not produced, is deficient, or is defective due to a change in the encoding gene. PIDs may result from a single genetic defect, but can be multifactorial. PIDs may be caused by recessive or dominant inheritance. In some examples, the subject who receives the recombinant granulocytes and/or recombinant lymphocytes has a PID. In some examples, the subject also as an acute or chronic infection, such as a bacterial, fungal, and/or viral infection. In some examples, the subject has autoimmune disease as a result of a PID. PIDs weaken the immune system, allowing repeated infections and other health problems to occur more easily. Examples of primary immunodeficiency diseases and their corresponding mutations include those listed in Al-Herz et al., Frontiers in Immunology, volume 5, article 162, Apr. 22, 2014, herein incorporated by reference. Specific examples are provided in Table 3.

TABLE 3
Exemplary PIDs and corresponding mutations

| | Genetic | |
|---|---|---|
| | Defect/Missing | |
| Disease | Protein | Exemplary Infections |
| Chronic granulomatous disease | NADPH oxidase, | Staphylococcus aureus, Serratia |
| (CGD) | one of 5 subunits: | marcescens, Burkholderia cepacia |
| | gp91phox | complex, Listeria, E. coli, |
| | p47phox | Klebsiella, Pseudomonas cepacia, |
| | p67phox | Nocardia, Aspergillus |
| | p22phox | |
| | p40phox | |
| X-linked immunodeficiency | MAGT1 | Epstein-Barr virus (EBV) |
| with magnesium defect, | | |
| Epstein-Barr Virus Infection | | |
| and Neoplasia (XMEN) | | |
| Autoimmune | TNFRSF6/CD95 | |
| lymphoproliferative syndrome | CASP10, CASP8, | |
| (ALPS) (FAS, FASLG, | FADD, PRKCD | |
| caspase 10, caspase 8, FADD | | |
| deficiency, PRKC8 deficiency) | | |
| Autoimmune polyglandular | Autoimmune regulator | Candida |
| syndrome type 1 (APS-1) | (AIRE) | |
| BENTA disease | CARD11 | |
| Caspase 8 deficiency state | Casp8 | Viral infections |
| (CEDS) | | |
| CARD9 deficiency | CARD9 | Candida |
| common variable | TNFRSF13B | bacterial or viral infections |
| immunodeficiency (CVID) | | of the upper airway, sinuses, |
| | | and lungs |
| Congenital | | |
| Neutropenia Syndromes | | |
| CTLA4 Deficiency | CTLA4 | |
| DOCK8 Deficiency | DOCK8 | recurrent viral infections of |
| | | the skin and respiratory system |
| GATA2 Deficiency | GATA2 | |
| Glycogen storage disease type | SLC37A4 or | |
| 1b | “G6PT1”, the G6P | |
| | transporter | |
| Glycosylation Disorders | | |
| With Immunodeficiency | | |
| Hyper-Immunoglobulin E | STAT3 | recurrent bacterial infections |
| Syndrome (HIES) | | of the skin and lungs |
| Hyper-Immunoglobulin M | CD40 ligand | respiratory infections, |
| (Hyper-IgM) Syndromes | | cryptococcal infections |
| Interferon Gamma, Interleukin | IFN-gamma, IL-12, | |
| 12, Interleukin 23 Deficiencies | IL-23 | |
| Leukocyte Adhesion | ITGB2 | |
| Deficiency (LAD) | | |
| LRBA Deficiency | LRBA | |
| PI3 Kinase Disease | PI3 Kinase | |
| PLCG2-associated Antibody | PLCG2 | |
| Deficiency and Immune | | |
| Dysregulation (PLAID) | | |
| Purine nucleoside | PNP | |
| phosphorylase (PNP) | | |
| deficiency | | |
| Severe Combined | adenosine deaminase | Candida, Pneumocystis jirovecii |
| Immunodeficiency (SCID) | (ADA), RAG1, RAG2, | |
| (such as ADA SCID, T-B+ | IL-2RG, JAK3, IL-2, | |
| SCID, T-B− SCID, IL-7 SCID) | -4, -7, -9, -15 or -21 | |
| Wiskott-Aldrich Syndrome | WAS | recurrent bacterial and fungal |
| (WAS) | | infections |
| X-Linked | XLA (Bruton tyrosine | infections of the ears, throat, |
| Agammaglobulinemia (XLA) | kinase or BTK) | lungs, and sinuses |
| | located on the X | |
| | chromosome | |
| X-Linked Lymphoproliferative | SH2D1A (SAP protein) | EBV |
| Disease (XLP) | | |

In some examples, the subject has one of the following PIDs resulting in combined T and B-cell immunodeficiencies: T−/B+ SCID (7c deficiency, JAK3 deficiency, interleukin 7 receptor chain α deficiency, CD45 deficiency, CD36/CD3F deficiency), T−/B− SCID (RAG 1/2 deficiency, DCLREC deficiency, adenosine deaminase (ADA) deficiency, reticular dysgenesis), Omenn syndrome, DNA ligase type IV deficiency, Cernunnos deficiency, CD40 ligand deficiency, CD40 deficiency, Purine nucleoside phosphorylase (PNP) deficiency, CD37 deficiency, CD8 deficiency, 1ZAP-70 deficiency, Ca++ channel deficiency, MHC class I deficiency, MHC class II deficiency, Winged helix deficiency, CD25 deficiency, STAT5b deficiency, Itk deficiency, DOCK8 deficiency, Activated PI3K Delta Syndrome, MALT1 deficiency, BCL10 deficiency, or CARD11 deficiency.

In some examples, the subject has one of the following PIDs resulting in an antibody deficiency: X-linked agammaglobulinemia (btk deficiency, or Bruton's agammaglobulinemia), μ-Heavy chain deficiency, 15 deficiency, Iga deficiency, BLNK deficiency, thymoma with immunodeficiency, common variable immunodeficiency (CVID), ICOS deficiency, CD19 deficiency, TACI (TNFRSF13B) deficiency, BAFF receptor deficiency, Hyper-IgM syndromes, heavy chain deletions, kappa chain deficiency, isolated IgG subclass deficiency, IgA with IgG subclass deficiency, selective immunoglobulin A deficiency, or Transient hypogammaglobulinemia of infancy (THI).

In some examples, the subject has one of the following PIDs: Wiskott-Aldrich syndrome, ataxia-telangiectasia, ataxia-like syndrome, Nijmegen breakage syndrome, Bloom syndrome, DiGeorge syndrome (when associated with thymic defects), cartilage-hair hypoplasia, Schimke syndrome, Hermansky-Pudlak syndrome type 2, Hyper-IgE syndrome, chronic mucocutaneous candidiasis, hepatic venoocclusive disease with immunodeficiency (VODI), or XL-dyskeratosis congenita (Hoyeraal-Hreidarsson syndrome).

In some examples, the subject has one of the following PIDs: Chediak-Higashi syndrome, Griscelli syndrome type 2, perforin deficiency, UNC13D deficiency, syntaxin 11 deficiency, X-linked lymphoproliferative syndrome, Autoimmune lymphoproliferative syndrome: type 1a (CD95 defects), type 1b (Fas ligand defects), type 2a (CASP10 defects), type 2b (CASP8 defects); APECED (autoimmune polyendocrinopathy with candidiasis and ectodermal dystrophy); IPEX (immunodysregulation polyendocrinopathy enteropathy X-linked syndrome); or CD25 deficiency.

In some examples, the subject has one of the following PIDs related to defects in phagocyte number/function: Severe Congenital Neutropenia: due to ELA2 deficiency (with myelodysplasia), Severe Congenital Neutropenia: due to GFI1 deficiency (with T/B lymphopenia), Kostmann syndrome, Neutropenia with cardiac and urogenital malformations, Glycogen storage disease type 1b, Cyclic neutropenia, X-linked neutropenia/myelodysplasia, P14 deficiency, Leukocyte adhesion deficiency type 1, Leukocyte adhesion deficiency type 2, Leukocyte adhesion deficiency type 3, RAC2 deficiency (Neutrophil immunodeficiency syndrome), Beta-actin deficiency, Localized juvenile periodontitis, Papillon-Lefevre syndrome, Specific granule deficiency, Shwachman-Diamond syndrome, Chronic granulomatous disease: X-linked, Chronic granulomatous disease: autosomal (CYBA), Chronic granulomatous disease: autosomal (NCF1), Chronic granulomatous disease: autosomal (NCF2), IL-12 and IL-23 β1 chain deficiency, IL-12p40 deficiency, 2Interferon 7 receptor 1 deficiency, Interferon 7 receptor 2 deficiency, STAT1 deficiency (2 forms), AD hyper-IgE, 2AR hyper-IgE, or pulmonary alveolar proteinosis.

In some examples, the subject has one of the following PIDs related to defects in innate immunity: Hypohidrotic ectodermal dysplasia (NEMO deficiency, IKBA deficiency); EDA-ID, IRAK-4 deficiency, MyD88 deficiency, Epidermodysplasia verruciformis, Herpes simplex encephalitis, chronic mucocutaneous candidiasis, or Trypanosomiasis.

In some examples, the subject has one of the following PIDs related to an autoinflammatory disorder: Familial Mediterranean fever, or TNF receptor associated periodic syndrome (TRAPS), Hyper-IgD syndrome (HIDS), CIAS1-related diseases (Muckle-Wells syndrome, Familial cold autoinflammatory syndrome, Neonatal onset multisystem inflammatory disease), PAPA syndrome (pyogenic sterile arthritis, pyoderma gangrenosum, acne), Blau syndrome, Chronic recurrent multifocal osteomyelitis and congenital dyserythropoietic anemia (Majeed syndrome), or DIRA (deficiency of the IL-1 receptor antagonist).

In some examples, the subject has one of the following PIDs related to a complement deficiency: Clq deficiency (lupus-like syndrome, rheumatoid disease, infections), Clr deficiency (idem), Cls deficiency, C4 deficiency (lupus-like syndrome), C2 deficiency (lupus-like syndrome, vasculitis, polymyositis, pyogenic infections), C3 deficiency (recurrent pyogenic infections), C5 deficiency (Neisserial infections, SLE), C6 deficiency (idem), C7 deficiency (idem, vasculitis), C8a deficiency, C8b deficiency, C9 deficiency (Neisserial infections), C1-inhibitor deficiency (hereditary angioedema), Factor I deficiency (pyogenic infections), Factor H deficiency (haemolytic-uraemic syndrome, membranoproliferative glomerulonephritis), Factor D deficiency (Neisserial infections), Properdin deficiency (Neisserial infections), MBP deficiency (pyogenic infections), MASP2 deficiency, Complement receptor 3 (CR3) deficiency, Membrane cofactor protein (CD46) deficiency, Membrane attack complex inhibitor (CD59) deficiency, Paroxysmal nocturnal hemoglobinuria, or Immunodeficiency associated with ficolin 3 deficiency.

Administration of the recombinant autologous granulocytes, recombinant autologous NK cells, and/or recombinant autologous lymphocytes can be used to treat any of these disorders. Treatment does not require 100% removal of all characteristics of the disorder, but can be a reduction in such. In one example the disclosed methods reduce the symptoms of an infection in the recipient subject (such as one or more of fever, large tender lymph nodes, throat inflammation, a rash, headache, sores of the mouth, nausea, vomiting, diarrhea, weight loss, viral load, ulcer size, size of infiltrate on imaging of lungs, brain, skin etc., blood parameters such as white cell count, inflammatory markers such as erythrocyte sedimentation rate, and C-reactive protein, and the like) for example a reduction of at least 10%, at least 20%, at least 50%, at least 70%, or at least 90% (as compared to no administration of the recombinant autologous granulocytes, recombinant autologous NK cells, and/or recombinant autologous lymphocytes).

In one example the disclosed methods increase the number of immune cells expressing the corrected protein (e.g., having functional NADPH oxidase activity), increase detectable protein expression (that was previously absent in the subject), increase the number of regulatory cells, increase immune competence (e.g., immunoglobulin class switch), increase the number of immune memory cells, and/or increase or improve the clinical status (e.g., weight, appetite, resolution of fevers), in the PID recipient subject, for example an increase of at least 5%, at least 10%, at least 20%, at least 50%, at least 70%, at least 90%, at least 100%, at least 200%, at least 500% or at least 1000% (as compared to no administration of the recombinant autologous granulocytes, recombinant autologous NK cells, and/or recombinant autologous lymphocytes).

### D. Additional Treatments

In some examples, in addition to receiving recombinant autologous granulocytes, recombinant autologous NK cells, and/or recombinant autologous lymphocytes, the treated subjects will receive additional therapy or treatment.

In one example, the subject is administered a therapeutically effective amount of an antibiotic, such as one or more of: chlortetracycline, amikacin, gentamicin, kanamycin, neomycin, netilmicin, tobramycin, paromomycin, streptomycin, spectinomycin, Geldanamycin, Herbimycin, Rifaximin, Loracarbef, Ertapenem, Doripenem, Imipenem/Cilastatin, Meropenem, Cefadroxil, Cefazolin, Cefalexin, Cefaclor, Cefprozil, Cefuroxime, Cefixime, Cefdinir, Cefditoren, Cefoperazone, Cefotaxime, Cefpodoxime, Ceftazidime, Ceftibuten, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin Erythromycin, Roxithromycin, Telithromycin, Spiramycin, Aztreonam, Furazolidone, Nitrofurantoin, Linezolid, Posizolid, Radezolid, Torezolid, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), Sulfonamidochrysoidine, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, or combinations thereof.

In one example, the subject is administered a therapeutically effective amount of an antiviral, such as one or more of: Abacavir, Acyclovir (Aciclovir), Adefovir, Amantadine, Amprenavir (Agenerase), Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Norvir, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine, or combinations thereof.

In one example, the subject is administered a therapeutically effective amount of an antifungal, such as one or more of: Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, amorolfin, butenafine, naftifine, terbinafine, Anidulafungin, Caspofungin, Micafungin, Aurones, Benzoic acid, Ciclopirox, Flucytosine or 5-fluorocytosine, Griseofulvin, Haloprogin, Tolnaftate, Undecylenic acid, Crystal violet, Balsam of Peru, Orotomide, Miltefosine, or combinations thereof.

In one example, the subject is administered an allogenic or autologous HSC transplant or a bone marrow transplant, for example following administration of the recombinant autologous granulocytes and/or recombinant autologous lymphocytes. Immunomodulation, the subject is administered a therapeutically effective amount of an interferon, such as interferon gamma-1b. In some examples, combinations of these additional therapies are administered. In one example, the subject has CGD and is also administered an antibiotic such as trimethoprim-sulfamethoxazole, an antifungal such as itraconazole, voriconazole, isovuconazole, terbinafine, amphotericin, or combinations thereof. In one example, the subject has CGD and is also administered a therapeutically effective amount of an interferon, such as interferon gamma-1b. In one example, the subject has XMEN and is also administered magnesium supplementation (e.g., oral magnesium threonate supplements). In one example, the subject has CTLA4 deficiency and is also administered sirolimus or orencia.

### EXAMPLES

### Example 1

CYBB Mutation Correction using CRISPR, a 53BPI Inhibitor, and a TP53 Inhibitor

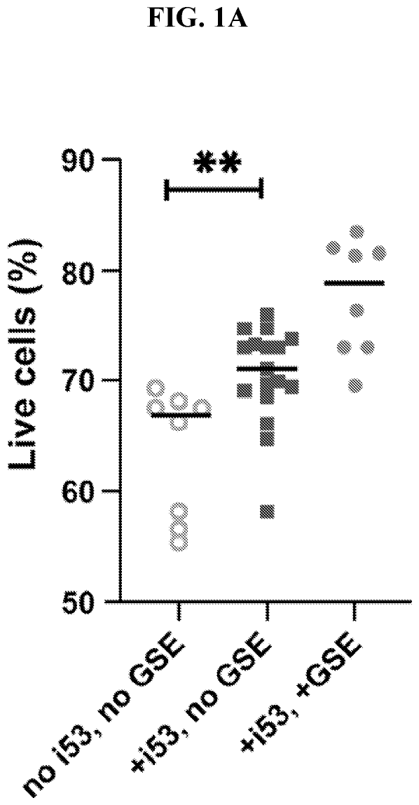

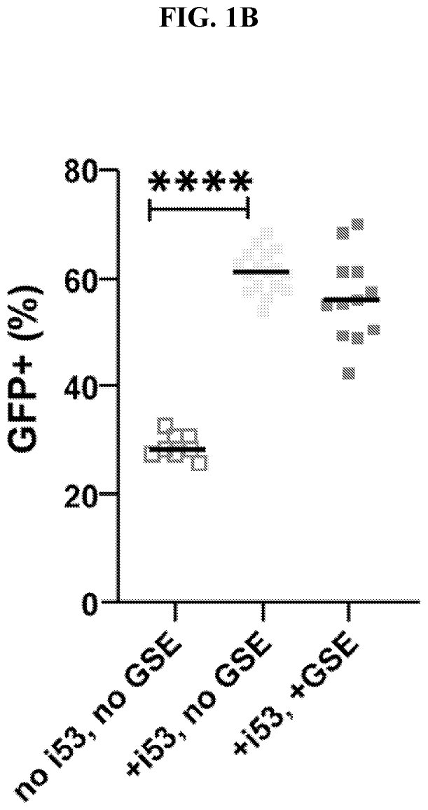

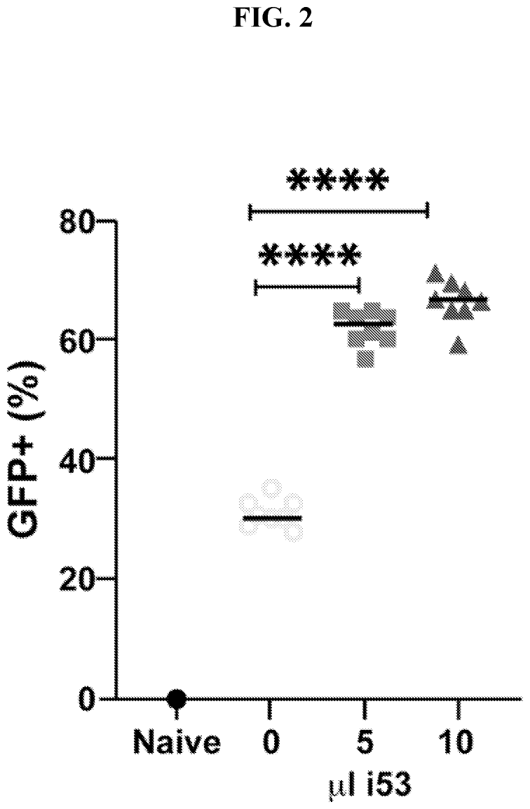

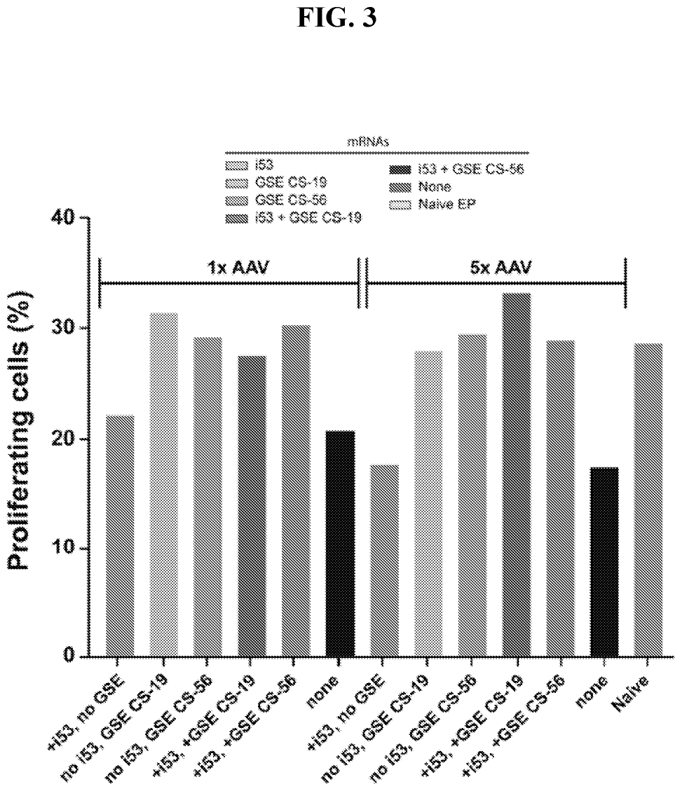

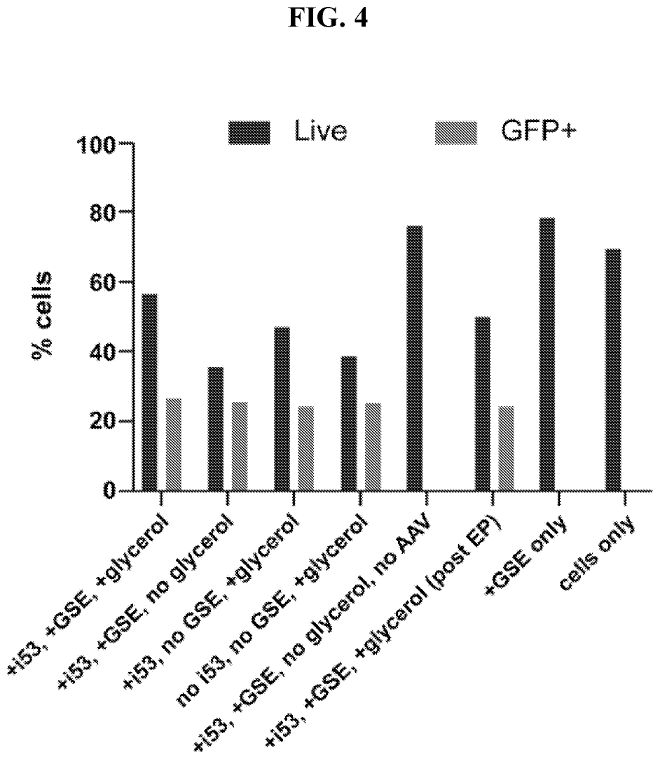

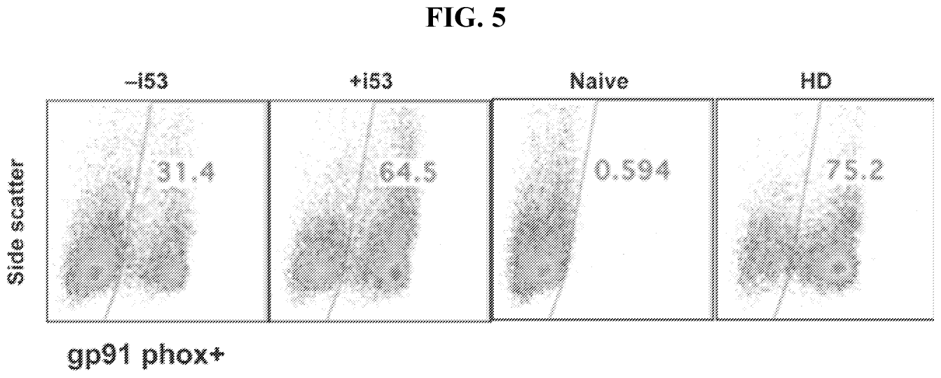

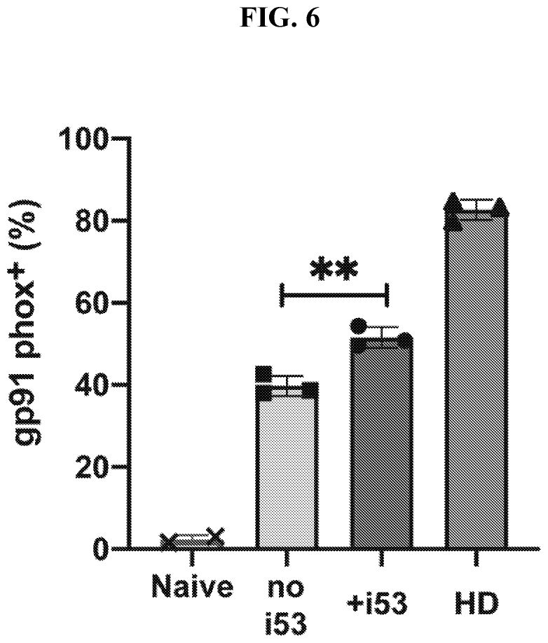

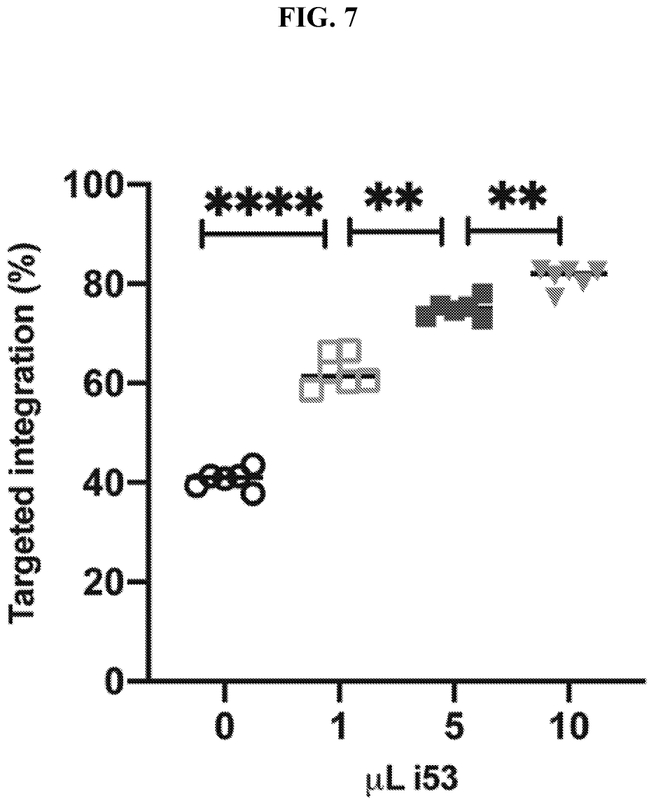

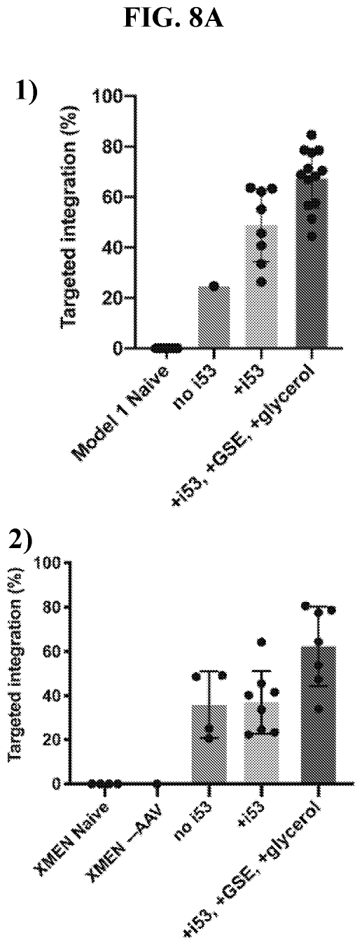

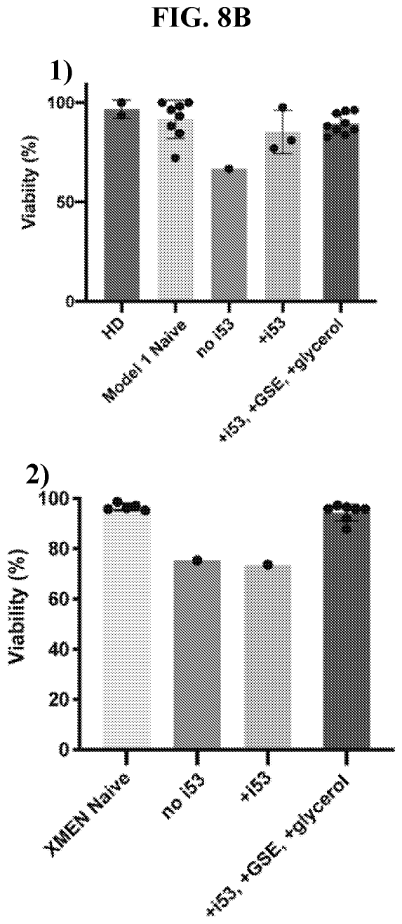

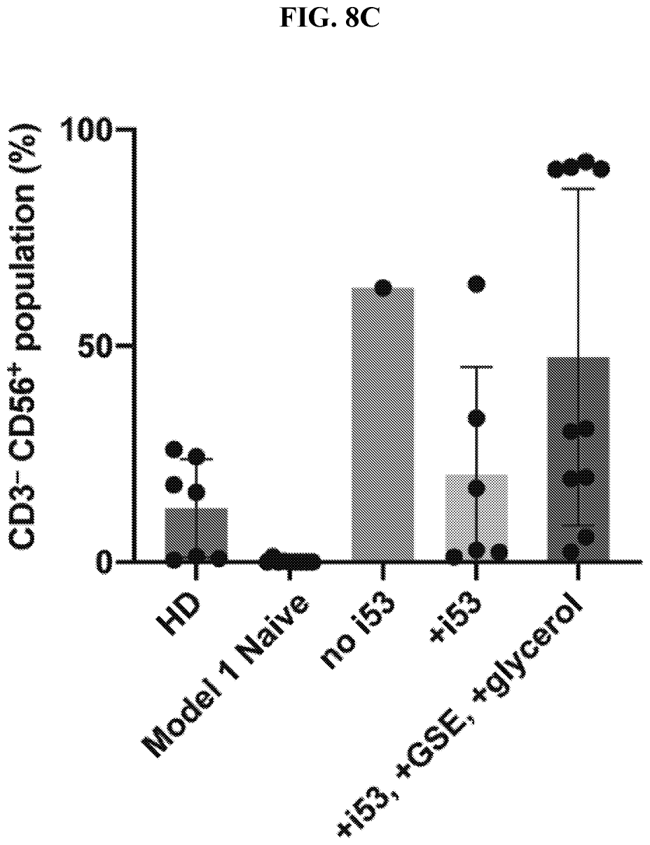

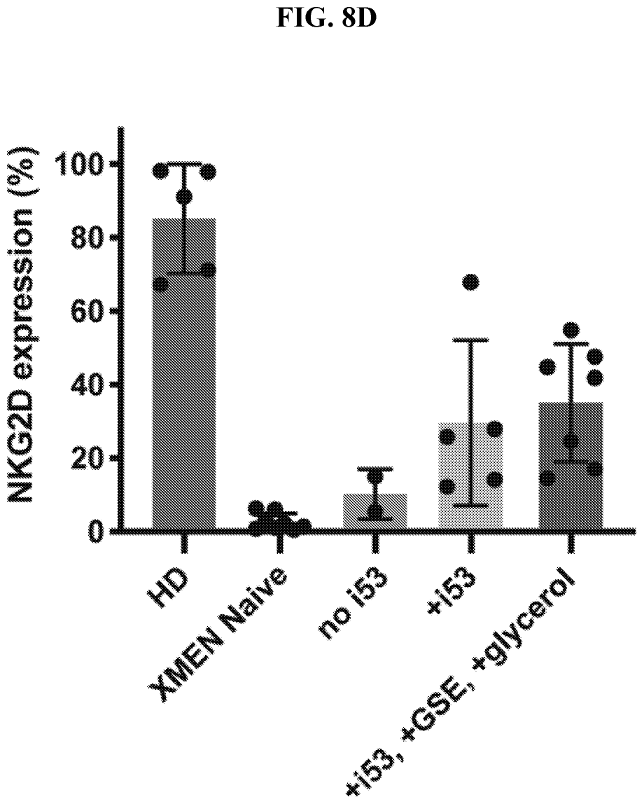

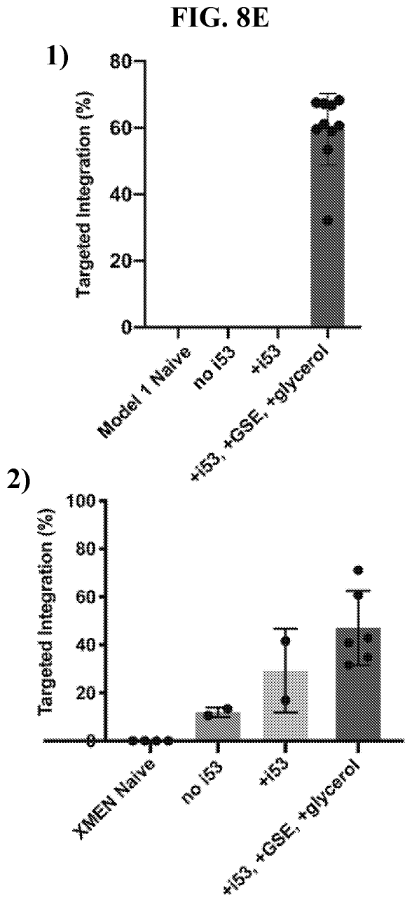

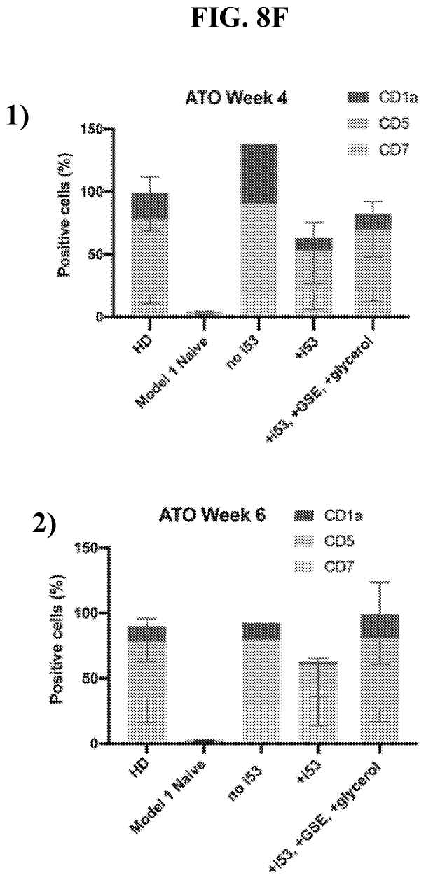

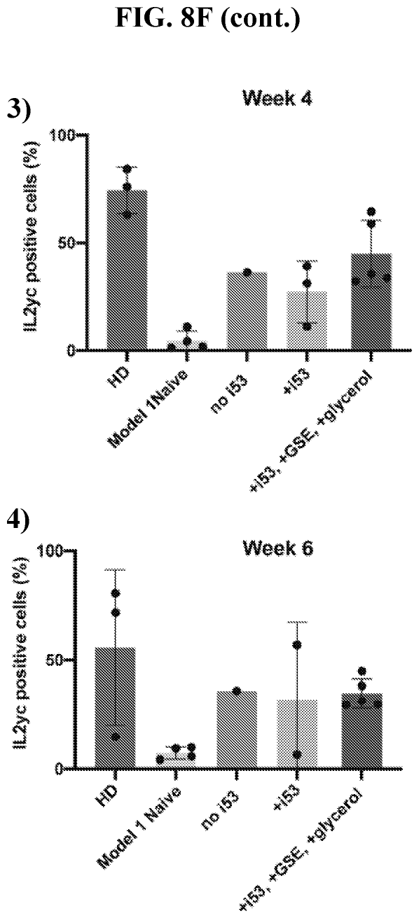

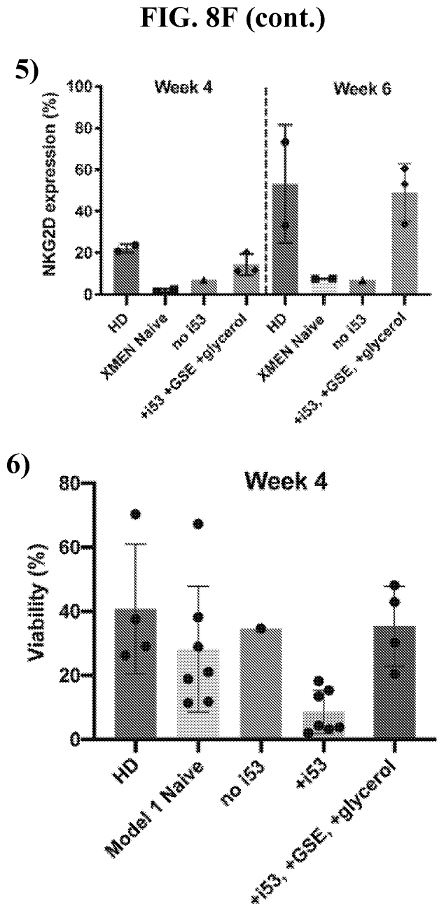

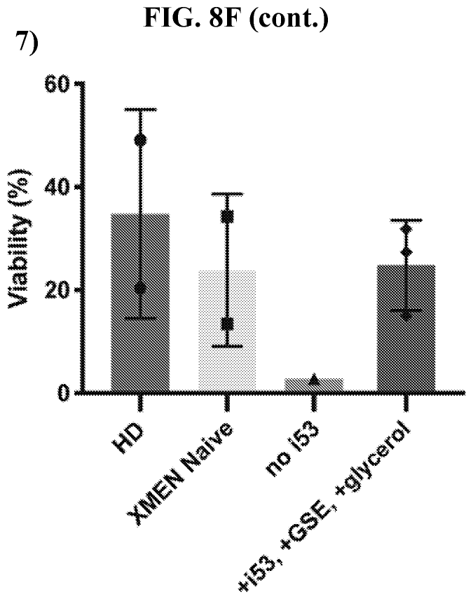

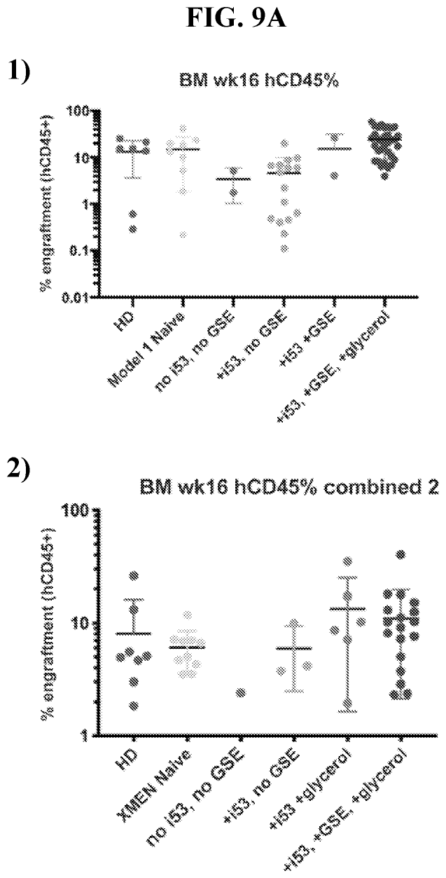

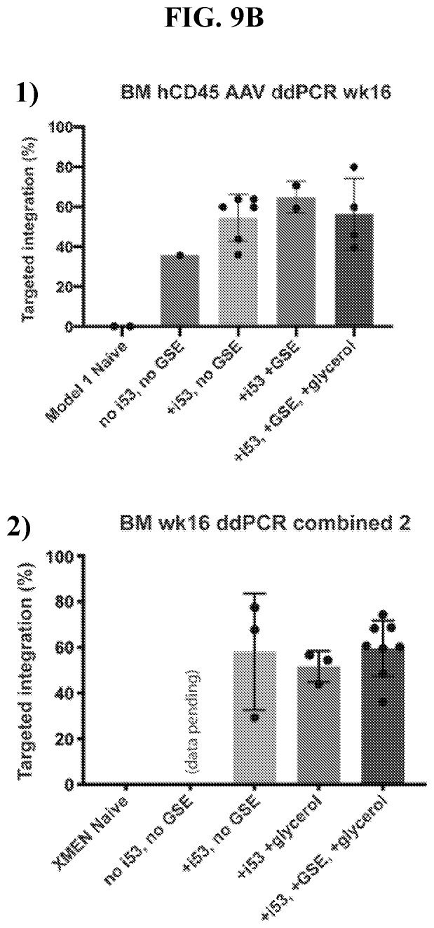

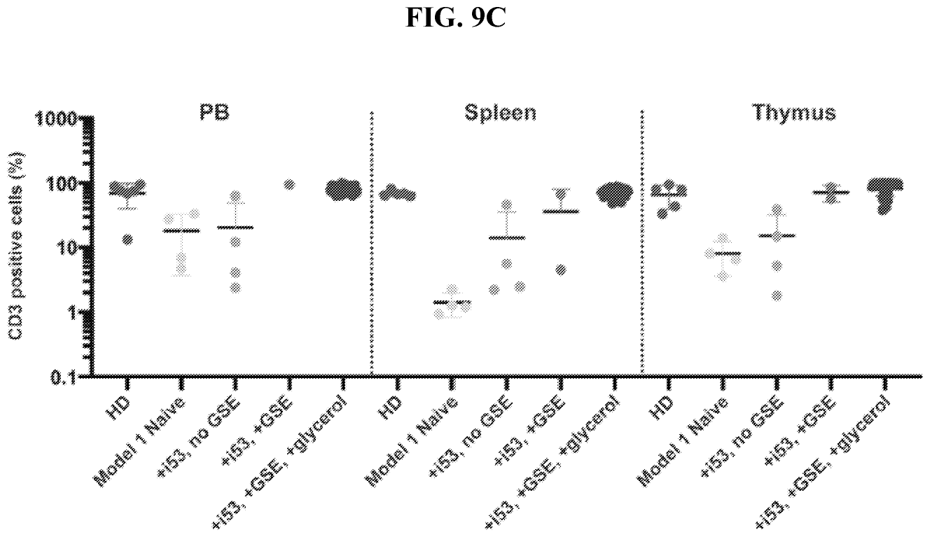

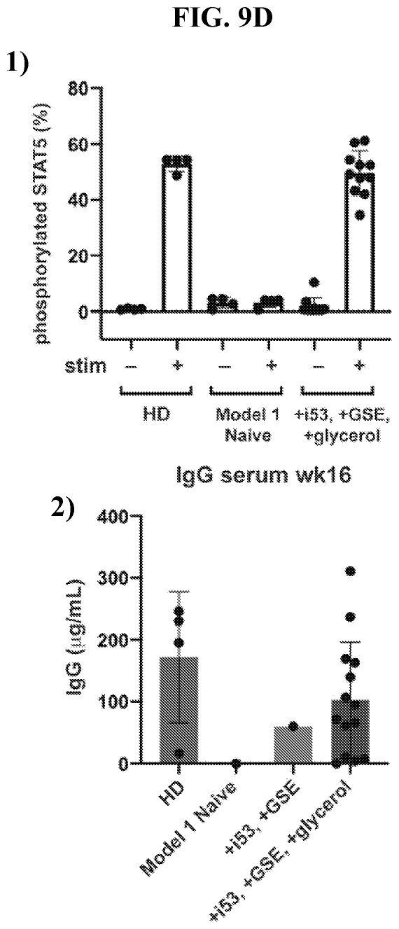

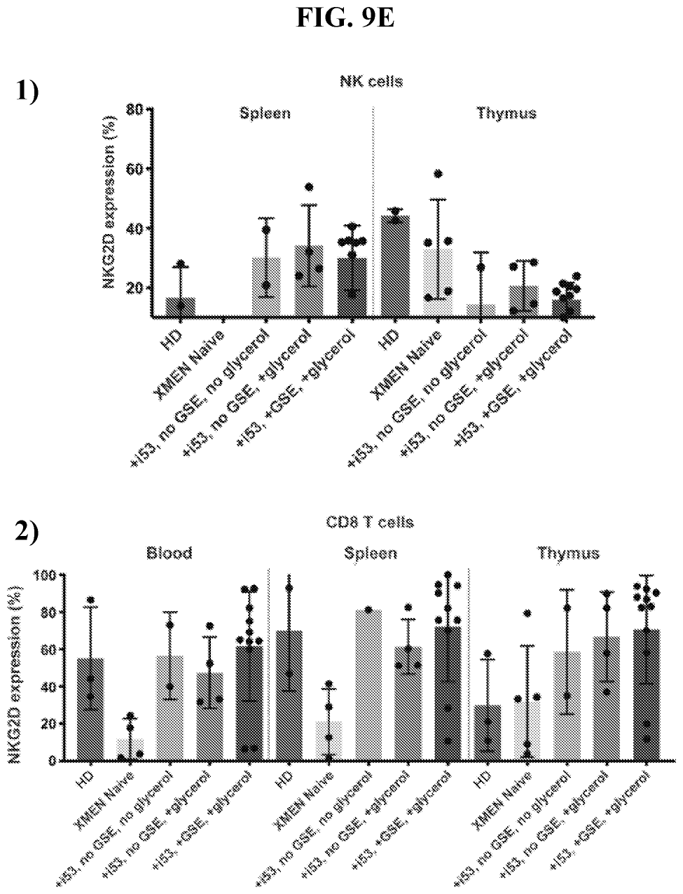

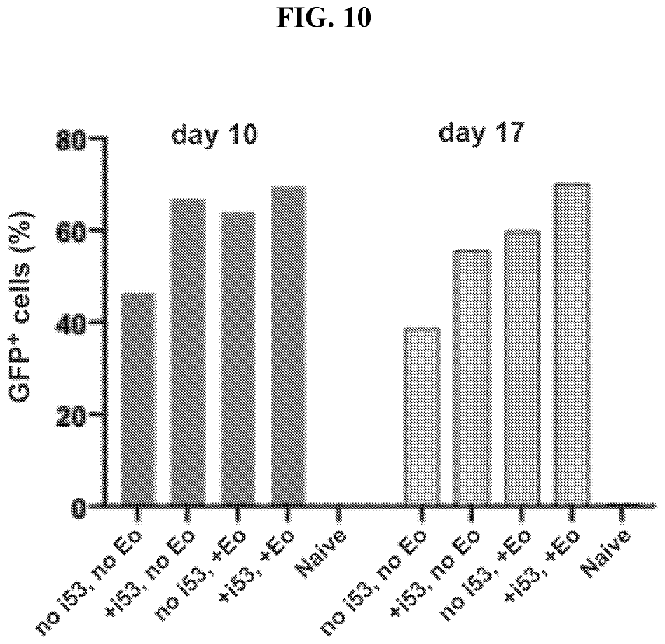

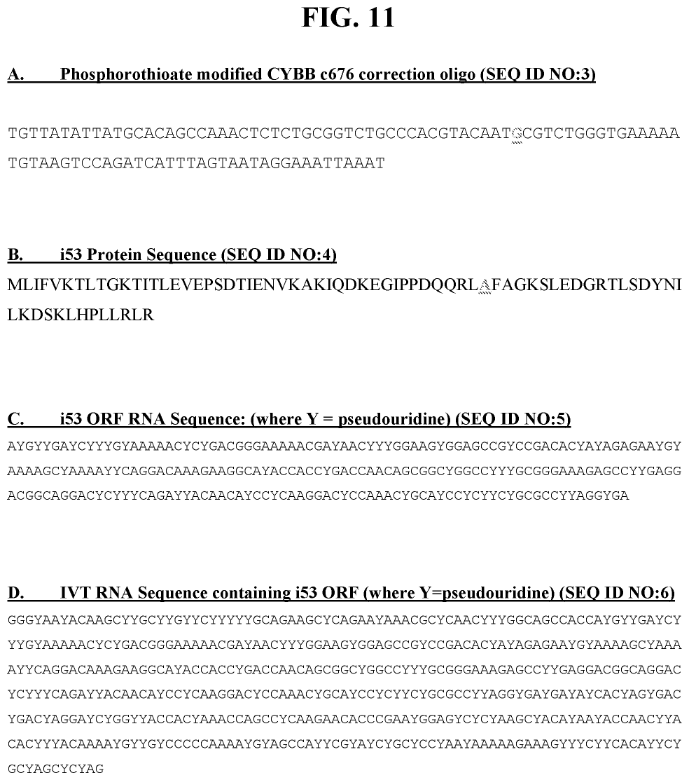

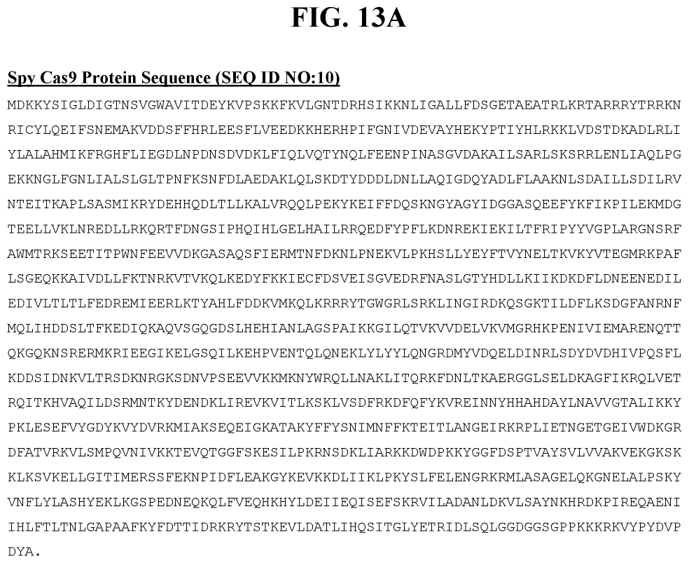

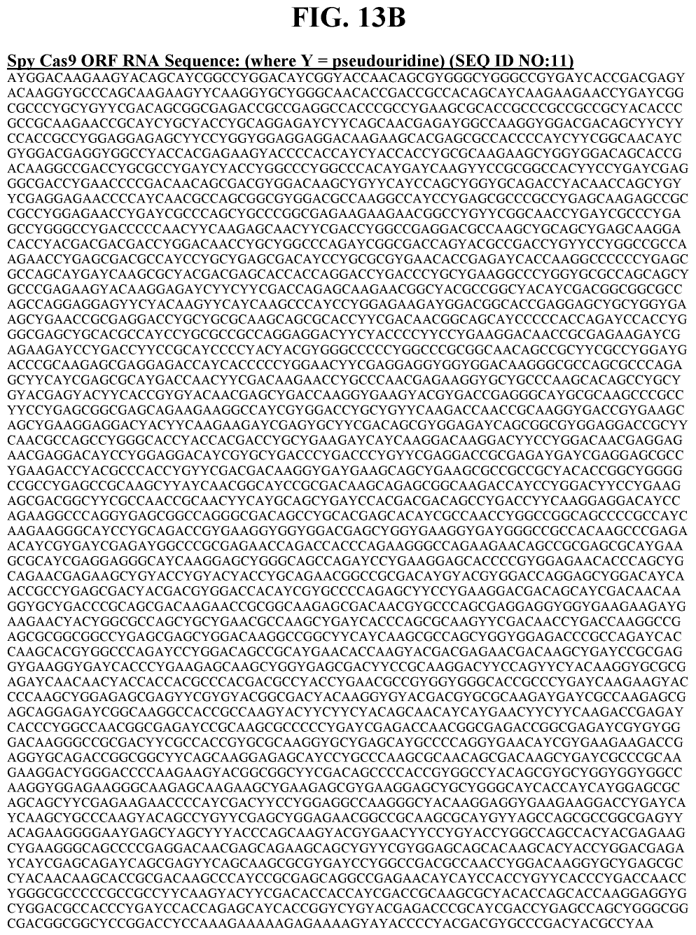

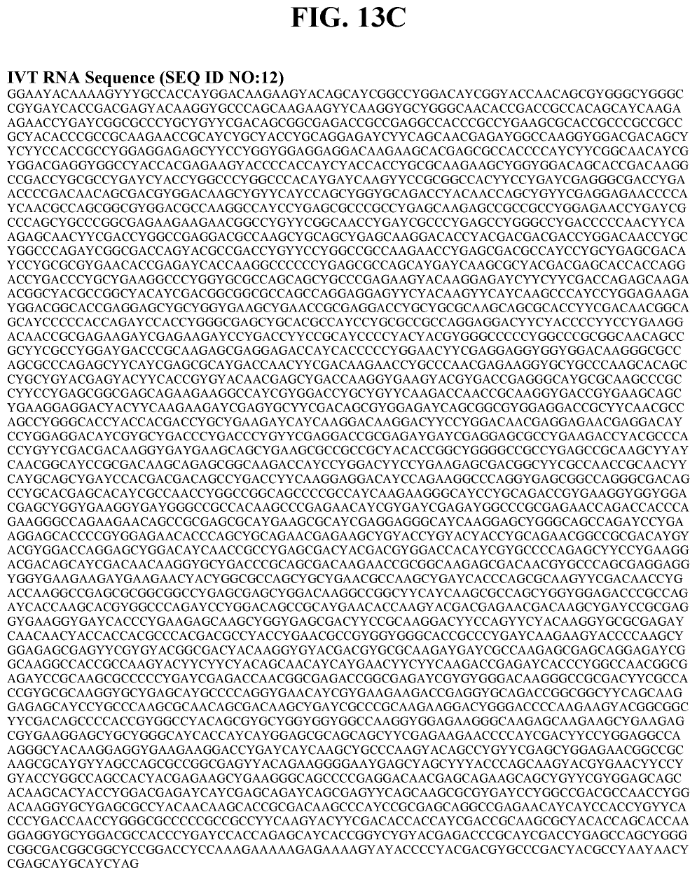

This Example describes how CYBB gp91 exon 7 C676 mutation-containing cells were CRISPR/Cas9 edited using an AAV-delivered, endogenous gp91 promoter-driven, GFP-expressing donor sequence also containing gp91 exons 7-13. Improved results were found by using mRNAs encoding Cas9, i53 (an inhibitor of the non-homologous end joining (NHEJ) domain of tumor suppressor p53 binding protein 1 (53BP1), which inhibitor is also referred to herein as “53 BP1 inhibitor”) and GSE CS-56 (aka GIS19), which is a TP53 inhibitor). The results and sequences employed are provided in FIGS. 1-13 and described in figure descriptions for FIGS. 1-13.

### Example 2

MAGT1 Mutation Correction using CRISPR, a 53BPI Inhibitor, and a TP53 Inhibitor

MAGT1 deficiency impairs glycosylation of proteins critical for immune function, including NKG2D, a receptor that permits CD8⁺ T and NK cells to recognize and kill virus-infected and transformed cells. Chronic EBV infection and lymphomas are major problems in patients with XMEN disease due to MAGT1 deficiency. A therapeutic option for MAGT1 deficiency is allogeneic stem cell transplant, which is associated with high mortality in this disease. This Example describes a CRISPR gene editing (GE) approach with CRISPR/Cas9/sg/AAV-MAGT1 supplemented with other factors (i53 (a BPI inhibitor), GSE CS-56 (a dominant negative TP53 inhibitor), glycerol) to enhance AAV-MAGT1-based homology-directed repair and cell fitness following GE. The data in this Example demonstrates highly efficient targeted insertion and functional correction of MAGT1 in engrafting CD34⁺ hematopoietic stem/progenitor cells for gene therapy, and in T cells for cellular therapy for treatment of infections and lymphoproliferative disease.

### Materials and Methods

### Human Samples

Cells from healthy volunteer donor (HD) and XMEN patient were obtained from the National Institutes of Health Department of Transfusion Medicine, Cell Processing Section, after written informed consent under auspices of National Institute of Allergy and Infectious Diseases (NIAID) institutional review board (approved protocols NIAID IRB 05-I-0213 and 94-I-0073). The conduct of these studies conforms to the Declaration of Helsinki protocols and all US federal regulations required for protection of human subjects. HD and XMEN patients underwent leukapheresis after CD34⁺ HSPC mobilization with granulocyte-colony stimulating factor (G-CSF; 15 mg/kg daily) for 5 days and plerixafor at 12 hours before blood collection. Apheresis allowed the separation of CD34⁺ HSPCs and CD34⁻ fraction (PBMCs) which were cryopreserved immediately after separation. Patient 1 (P1) carries a hemizygous mutation in Exon 4 of the MAGT1 gene: g.43183delC (c.598delC, p.Arg200Glyfs*13) and was previously described in Li et al. as patient E1 (46).

Design of sgRNA

Ten candidate sgRNAs with 2′-O-methyl 3′-phosphorothioate modifications at the first 3 and last 3 nucleotides targeting Exon 1 of the MAGT1 gene were purchased from Synthego (Menlo Park, CA). The sgRNA spacer sequences and number of off-targets (OTs) are provided in Table 1.

rAAV6-MAGT1 donor design and production

The recombinant adeno-associated virus serotype 6 (rAAV6)-MAGT1 donor plasmid was designed as following: codon-optimization of MAGT1 cDNA (Integrated DNA Technologies (IDT) optimization tool), WPRE enhancer sequence and B-globin polyA tail, flanked by 0.4-kb homology arms. Synthesis of this plasmid was performed by IDT (Coralville, IA). Restriction sites before the LHA (NotI) and RHA (XhoI) were used for cloning into a pUC57 inverted terminal repeat (ITR)-containing AAV vector genome donor plasmid. This plasmid was used for large-scale production of rAAV6-MAGT1 donor (Vigene Biosciences, Inc, Rockville, MD).

### Gene Editing of T Cells

Frozen PBMCs were thawed and stimulated with MACS® GMP T Cell TransAct™ (Miltenyi Biotec) for 3-4 days in ImmunoCult™-XF T Cell Expansion Medium (STEMCELL™ Technologies) supplemented with 500 IU/mL rhIL-2 and 5 ng/mL rhIL-7 and rhIL-15 (PeproTech) before transfection. One million T cells were electroporated in 20 μL Opti-MEM buffer with 25 μg/mL Spy Cas9 mRNA (CELLSCRIPT, LLC), 200 μg/mL sgRNA (Synthego), 100 μg/mL i53 mRNA (CELLSCRIPT, LLC) and 2% glycerol (Sigma-Aldrich) with Amaxa4D (Lonza Nucleofector) program EO-115 immediately followed by rAAV6-MAGT1 transduction. Medium was changed every 3-4 days with 500 IU/mL rhIL-2 and cell concentration maintained at a density of 1-2×10⁶ cells/mL.

### Gene Editing of CD34⁺ HSPCs

Cryopreserved CD34⁺ HSPCs were thawed (day 0) and cultured in StemSpan SFEM II medium (STEMCELL™ Technologies) supplemented with 100 ng/mL stem cell factor (SCF), Flt3 ligand, thrombopoietin (Peprotech), 0.75 μM of StemRegenin-1 (STEMCELL™ Technologies) and 35 nM of UM171 (STEMCELL™ Technologies) for 2 days (37). On day 2, cells were washed in PBS and resuspended in HyClone™ MaxCyte® electroporation buffer (at 2-5×10⁷ cells/mL) electroporated with 25 μg/mL Spy Cas9 mRNA, 200 μg/mL single-guide RNA (sgRNA; Synthego), 100 μg/mL i53 mRNA, 650 pmol/mL GSE CS-56 mRNA and 2% glycerol with MaxCyteGT electroporation device (MaxCyte, Gaithersburg, MD) program HSPC_3_OC immediately followed by rAAV6-MAGT1 transduction. Electroporated cells were cultured in the same medium for 2 days. At 48 h post-EP, the cells were harvested and counted by hemocytometer with trypan blue to determine viability before their use for molecular analysis, phenotypic analysis by flow cytometry, CFU assay, T or NK cell differentiation or cryopreservation for transplant studies.

### T Cell Differentiation: Artificial Thymic Organoid (ATO) System

CD34⁺ HSPCs were differentiated into T cells in vitro using a 3D artificial thymic organoid system (26, 27). Briefly, CD34⁺ cells were co-cultured with MS5 murine stromal cell line modified to express DLL4 ligand in a volume of 5 μL on 0.4 m transwell inserts (EMD Millipore). 1 mL of medium containing 5 ng/ml rhFLT3L and 5 ng/ml rhIL-7 (Peprotech) was added per well and medium changed every 3-4 days. After 4 and 6 weeks, ATOs were harvested by disaggregation by pipetting with FACS buffer and filtered through a 70 m cell strainer before staining for flow cytometry analysis.

### NK Differentiation

CD34⁺ HSPCs were differentiated into NK cells in vitro using a protocol adapted from (47). 200,000 CD34⁺ cells were resuspended in IMDM supplemented with 20% FBS and 50 μM BME containing 5 ng/mL rhIL-3 (first week only), 10 ng/mL IL-15, 20 ng/mL IL-7, 20 ng/mL SCF, 10 ng/mL Flt3L and 100 U/mL IL2 for 35 days. K562-mb15-41BBL cells (kindly provided by St Jude Children's Research Hospital, Memphis, TN) (28, 29) were irradiated (40 Gy) and added at day 7 and day 21 of differentiation. Cells were harvested at day 35 of differentiation for flow cytometry analysis, cytotoxicity assay and gDNA extraction.

### Cytotoxicity Assay

NK cell cytotoxicity was determined as follows. Briefly, target cells (K562) were labeled with the fluorescent target-cell marker TFL4 (1:4000, Oncolmmunin, USA) for 20 min at 37° C. in PBS, then washed in complete RPMI 1640 medium. NK cells (purity determined based on the percentage of CD3⁻CD56⁺ population prior to the assay) were seeded into round bottom 96-well plates at various Effector-to-Target (E:T) ratios up to 2 in RPMI medium supplemented with 100 IU/mL IL2 and 10 ng/mL IL15. Plates were spun at 100 g for 5 min followed by incubation for 5 hours at 37° C. After 5 h, supernatant was removed, and cells were stained with propidium iodide (20 ng/well; Sigma-Aldrich). Samples were analyzed by flow cytometry (FACS Canto, BD Biosciences). The percentage of target cells with PI staining was analyzed in the tracking dye TFL4-positive K562 cell population.

### Transplantation Studies

The use of triple transgenic immunodeficient mice NOD.Cg-Prkd^{scid} Il2rg^{tm1Wjl} Tg (NSG-SGM3 or NSGS) expressing human IL3, GM-CSF and SCF cytokines (30) (Stock No: 013062; The Jackson Laboratory) for transplantation studies was approved by NIAID Institutional Animal Care and Use Committee under the animal use protocol LCIM-1E. The conduct of these studies conforms to AAALAC International guidelines and all US federal regulations required for protection of research animals. Newborn mice (0-4 days old) were irradiated at 100 cGy and immediately intrahepatically injected (courtesy of Mara Pavel-Dinu) with 1-1.5×10⁶ viable CD34⁺ cells per mouse. Peripheral blood from tail vein was analyzed at weeks 12 and 16 after transplantation. At 16 weeks, mice were sacrificed, and bone marrow, spleen and thymus were harvested for analysis of engraftment, sorting or gDNA extraction as indicated.

### Flow Cytometry

Briefly, cells were washed in FACS buffer (PBS with 0.1% bovine serum albumin) and centrifuged for 5 minutes at 1800 rpm. Cells were incubated at RT for 30 minutes and washed in FACS buffer. Flow cytometry analysis was performed using a BD Canto flow cytometer, DIVA software (BD Biosciences) and FlowJo analysis software (Tree Star).

The following antibodies were used for T cells and NK cells in vitro analysis: PE-conjugated anti-CD56 (clone 3G8, BD Pharmingen), PerCP-Cy5.5-conjugated anti-CD3 (clone HIT3a, BioLegend), APC/Cy7-conjugated anti-CD8 (clone SK1, BioLegend), FITC-conjugated anti-CD4 (clone A161A1, BioLegend), APC-conjugated NKG2D (clone 1D11, BioLegend), PE-conjugated anti-CD28 (clone CD28.2, BioLegend), APC-conjugated anti-CD70 (clone 113-16, BioLegend).

Antibodies used for ATO analysis are as follow: eF450-conjugated CD14 (clone 61D3, eBioscience), PE-Cy7-conjugated CD5 (clone UCHT2, Invitrogen), AF700-conjugated CD7 (clone 124-1D1, Invitrogen), FITC-conjugated CD56 (clone MEM-188, BioLegend), PE-conjugated CD34 (clone 581, Biolegend), APC-conjugated CD1a (clone HI149, BioLegend), V500-conjugated CD45 (clone HI30, BD Bioscience), PerCP-Cy5.5-conjugated CD19 (clone HIB19, BioLegend), BV421-conjugated CD3 (clone UCHT1, BD Biosciences), AF700-conjugated CD4 (clone OKT4, Invitrogen), PE-Dazzle-conjugated CD8a (clone RPA-T8, BioLegend), APC-conjugated TCRab (clone IP26, Invitrogen).

Antibodies used for in vivo studies are as follow: PE-conjugated and FITC-conjugated CD45 (clone HI30, BD Bioscience), APC-conjugated CD33 (BD Biosciences), APC-Cy7-conjugated and PE-Cy7-conjugated CD3 (clone HIT3a, BioLegend), PerCP-Cy5.5-conjugated CD19 (clone HIB19, BioLegend), PE-Cy7-conjugated CD34 (clone 561, BioLegend), PerCP-Cy5.5-conjugated CD4 (clone RPA-T4, BioLegend), APC/Cy7-conjugated anti-CD8 (clone SK1, BioLegend), APC-conjugated CD56 (clone 5-1H11, BioLegend), PE-conjugated NKG2D (clone 1D11, BioLegend).

### Cell Sorting

Spleen samples were washed and surface staining for cell sorting was performed by pelleting cells and resuspending in 100 l of FACS buffer for 25 min at 4° C. in the dark with the following antibodies: PE-conjugated CD45 (BD Biosciences), APC-conjugated CD33 (BD Bioscience), APC-Cy7-conjugated CD3 (BioLegend), BV421-conjugated CD19 (BioLegend) and FITC-conjugated CD56 (BioLegend). Cells were washed once in FACS buffer and filtered through a 40-μm filter before resuspension in HBSS with 5% FBS with 7-AAD staining in order to exclude dead cells. Cells were sorted using a FACS-Aria Illu cell sorter (BD Biosciences) at the Flow Cytometry Section (Research Technologies Branch, DIR, National Institute of Allergy and Infectious Diseases, NIH).

Detection of on-Target Activity and Targeted Integration

gDNA extraction was performed on CD34⁺ cells, NK or T cells using QIAamp® DNA mini kit (QIAGEN) according to manufacturer's instructions. Detection of indels at MAGT1 exon 1 locus (on-target cutting efficiency) was measured by TIDE (Tracking of Indels by Decomposition) method (48, 49) after amplification of Exon1 of the MAGT1 gene using primers Fwd-prom 5′-GACCAATGAAAACGCTCCAG-3′ (SEQ ID NO:40 and Rev-Intl 5′-ATCGGCCAGCAGTATAGAGTC-3′ (SEQ ID NO:41), and Sanger sequencing by the CCR Genomics Core, NCI, NIH. The type and frequency of events at the cutting site were identified using the ICE v2 CRISPR analysis tool provided by Synthego website.

Targeted insertion (TI) of MAGT1 cDNA was quantified by digital droplet PCR (ddPCR) using one primer located upstream of the LHA (5′-GAGGCAGGATAACGGACTAAAT−3′, SEQ ID NO:42) the second primer (5′-TCCACCTAGCGGCCATATTA-3′, SEQ ID NO:43) inside the cDNA and a probe (5′-AAAGTATCCTTGATTGAGGGCCGGG-3′, SEQ ID NO:44).

### Identification and Quantification of Off-Targets (OTs)

Experimental identification of off-targets sites was performed in vitro on male HD CD34⁺ cells using Circularization for High-throughput Analysis of Nuclease Genome-wide Effects by sequencing, or CHANGE-seq, for large-scale genome-wide profiling of off-targets (34).

In silico computational analysis was performed using predictive tools like COSMID (CRISPR Off-target Sites with Mismatches, Insertions, and Deletions; (31)), Cas-OFFinder (32)), CCTop ((33)) and CRISPR-OFF webserver ((50)). The location of the top eight OT loci and primers used for quantification were listed in Table 2.

### Statistical Analysis

Data were expressed as means±SD. Differences were tested using a student's t test (2 groups), or a one-way ANOVA (>2 groups) and Tukey's post hoc multiple comparisons test. Spearman's correlation test was used to analyze the association between parameters. Statistical analyses were performed using GraphPad Prism (version 8.1.0). Significances were indicated as *p<0.05, **p<0.01, ***p<0.001 and ****p<0.0001, n.s non-significant.

### CFU Assay

1,000 CD34⁺ cells were resuspended in IMDM+2% FBS and added to a 3-mL aliquot of Methocult H4435 Enriched methylcellulose (STEMCELL™ Technologies), briefly vortexed, and 1.5 mL methylcellulose was dispensed per 35-mm dish in duplicate. After 14 days of culture at 37° C. and 5% CO2, the different types of hematopoietic colonies were identified according to morphological criteria and scored.

### CFSE Proliferation Assay

The following day after EP, CD34⁺ cells were stained with CellTrace™ CFSE kit per manufacturer's instructions (ThermoFisher Scientific) and analyzed by flow cytometry 5 days later. Determination of the proliferation index was done using FlowJo software.

### Western Blot

Western blot analysis was performed using primary antibodies directed against human MAGT1 protein (1:100; rabbit polyAb; Proteintech) or b-actin (1:2,500; rabbit polyAb; Abcam) and secondary HRP conjugate anti-rabbit IgG (1:1000; SantaCruz Biotechnology). Revelation was performed using SuperSignal™ West Dura Extended Duration Substrate (ThermoFischer Scientific) with the BioRad chemiluminescent camera (BioRad).

### Results

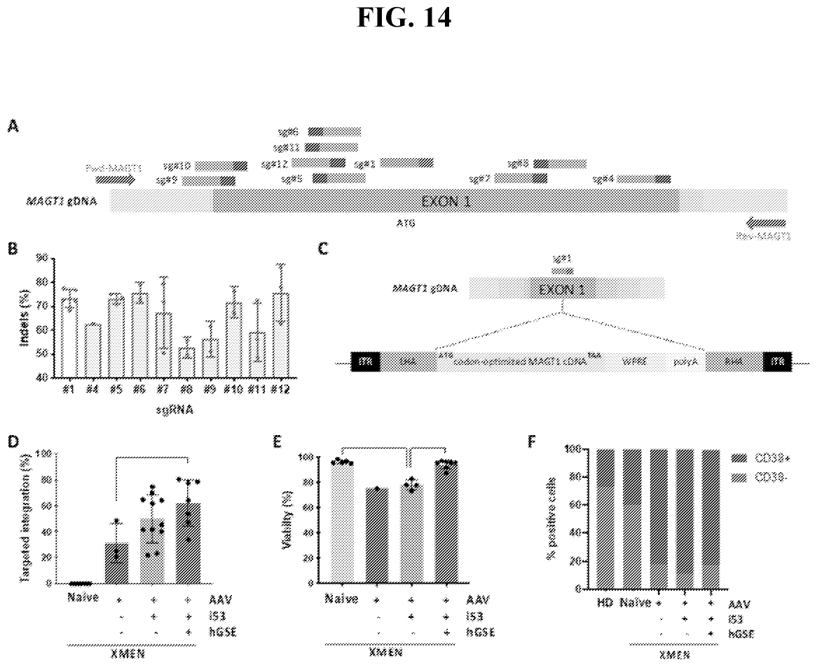

Optimization of TI efficiency in XMEN CD34⁺ HSPCs using gene editing enhancing factors First, we designed 10 single guide RNAs (sgRNAs) using online tools targeting nearby the MAGT1 gene transcription start site (TSS) (FIG. 14A). Experiments in CD34⁺ cells revealed efficient genome editing efficiencies (>70%) with five of the sgRNAs (FIG. 14B); sgRNA #1 was selected for the remaining studies because of its proximity to the ATG start codon (6 bp downstream) and a predicted highly specific profile (Table 1).

TABLE 1

| | | | | | Geonomic sites with | |
|---|---|---|---|---|---|---|
| | sgRNA spacer sequence | | | Distance | ‘n’ mismatches (0, 1, | |
| Name | (5′→3′) | PAM | Strand | from ATG | 2, 3, 4 mismatches) | SEQ ID |
| sgRNA#1 | GAGCGAACATGGCAGCGCGT | TGG | + | ATG +6 | 1.0,0,1.44 | 14 |
| sgRNA#4 | AGCCTCTGCCCAAAGAAAGA | AGG | + | ATG +91 | 1.1,1.35,270 | 15 |
| sgRNA#5 | AGTGAAACTTTGCTCCGGCT | AGG | − | ATG −39 | 1.0,1.6,49 | 16 |
| sgRNA#6 | CTTTGCTCCGGCTAGGTCTG | AGG | AGG | ATG −46 | 1.0,0.52,219 | 17 |
| sgRNA#7 | GTGTGTCTCTGTGACCATGG | TGG | + | ATG +37 | 1.0,1.31,325 | 18 |
| sgRNA#8 | GATGAGCAGCGCCACCACCA | TGG | AGG | ATG +40 | 1.0,1.8,146 | 19 |
| sgRNA#9 | TGGACCAATGAGGAAAGGCA | AGG | + | ATG −86 | 1.0,3.23,253 | 20 |
| sgRNA#10 | GACCAATGAGGAAAGGCAAG | GGG | + | ATG −84 | 1.1,1.20,319 | 21 |
| sgRNA#11 | TTTGCTCCGGCTAGGTCTGA | GGG | AGG | ATG −47 | 1.0,0.6,55 | 22 |
| sgRNA#12 | GCCCCACCCTCAGACCTAGC | CGG | AGG | ATG −42 | 1.0,2.28,372 | 23 |

Table 1 provides a list of sgRNAs screened for targeting Exon 1 of MAGT1 gene. Predictions for OT sites were generated using Synthego online tool using Homo sapiens GRCh38 reference genome and PAM NGG only. ‘1’ for the 0 mismatch accounts for the on-target site.

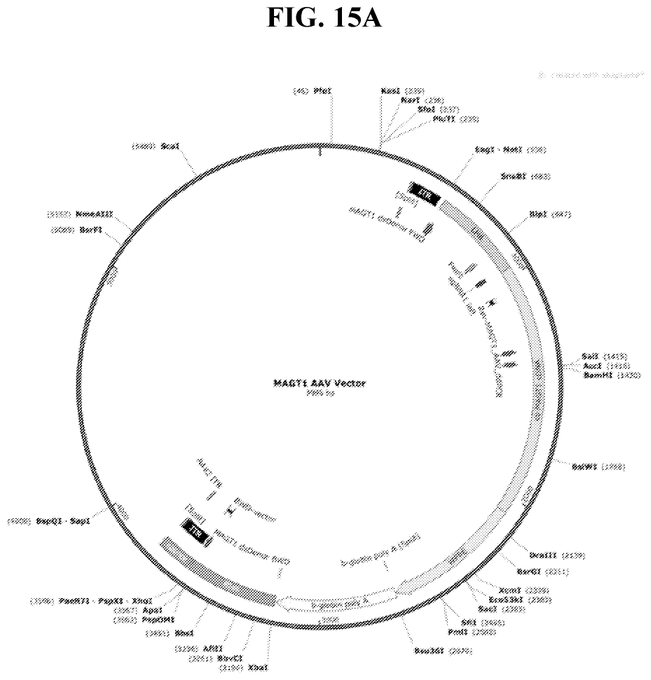

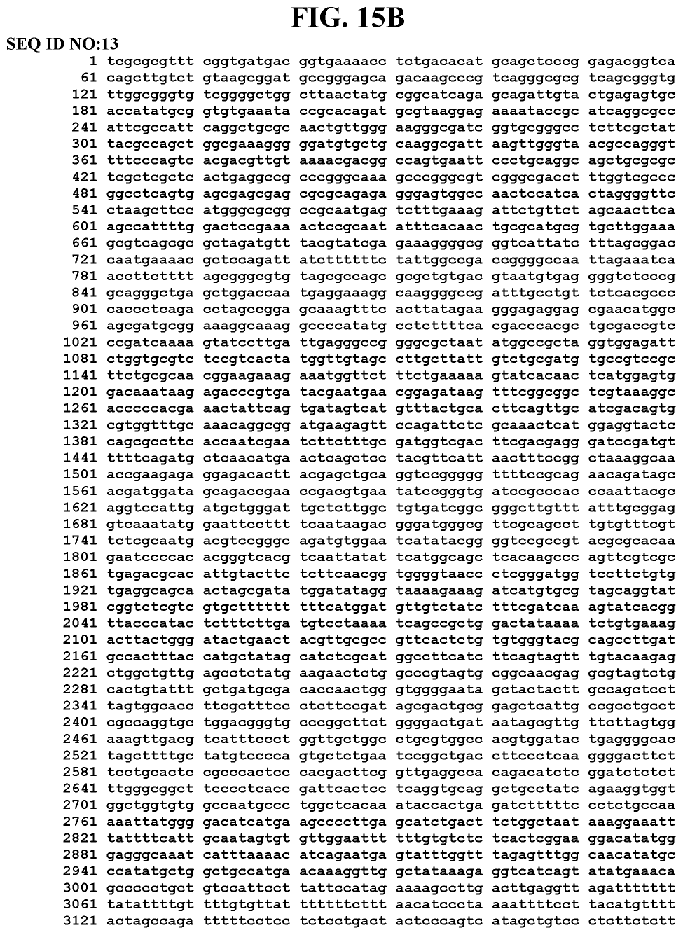

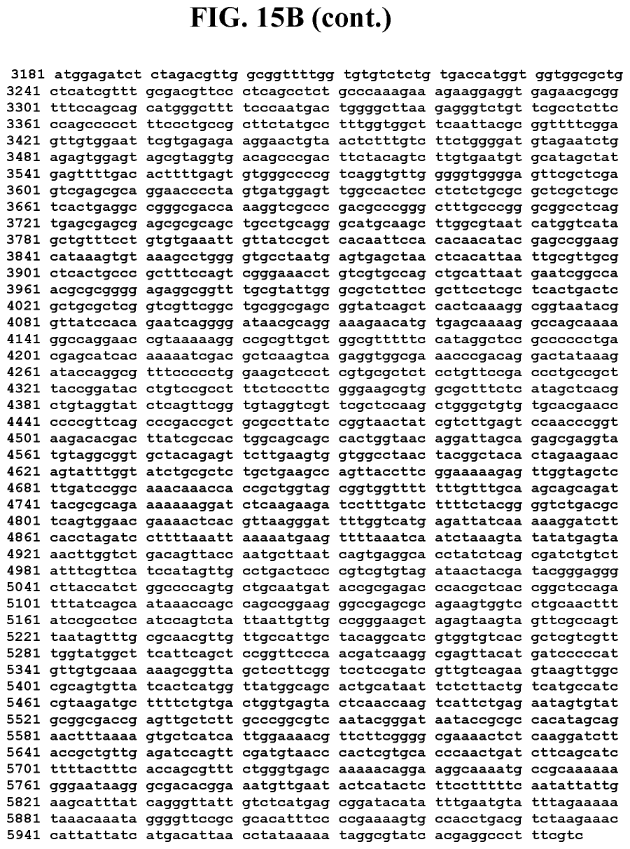

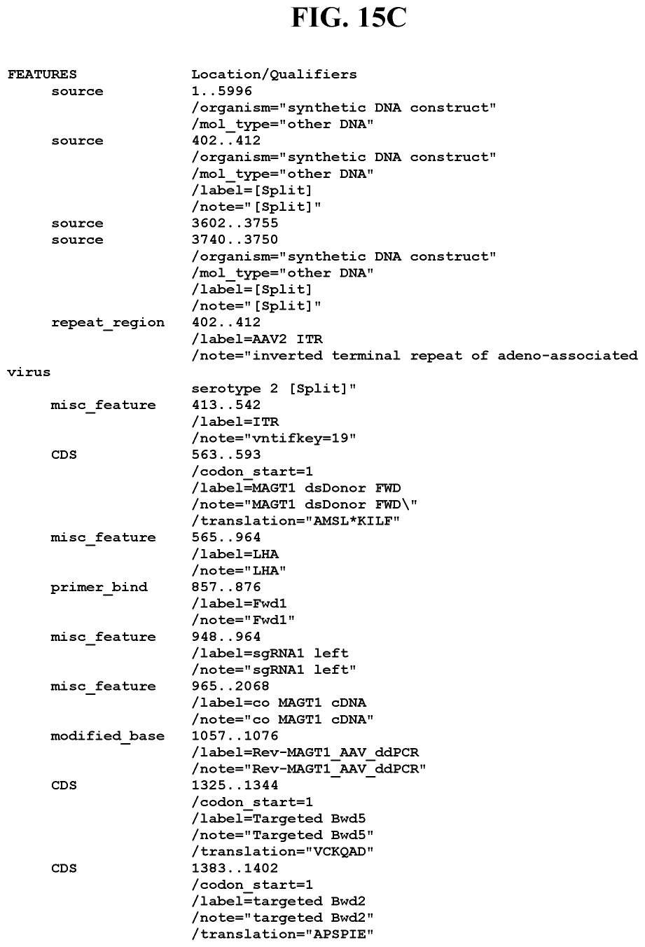

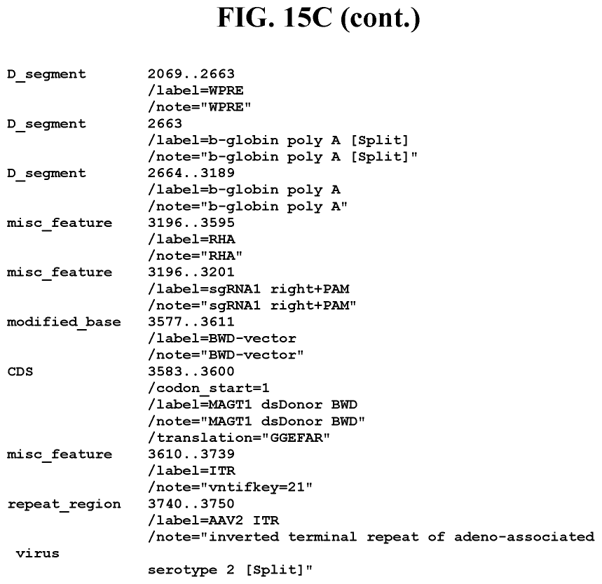

The gene editing components (Cas9 mRNA and sgRNA) were delivered by electroporation (EP) into healthy donor (HD) CD34⁺ HSPCs after 2 days of stimulation (Stem cell factor, FLT−3 ligand, Thrombopoietin at 100 μg/mL each). Peripheral blood CD34+ HSPCs from HD controls and a XMEN patient (P1, null mutation c.598delC, pArg200Glyfs*13) were collected following informed consent and cryopreserved. We designed a corrective MAGT1 donor encoding: (1) a ˜3 kb-codon-optimized MAGT1 cDNA, (2) a woodchuck hepatitis virus posttranscriptional regulatory elements (WPRE) to improve mRNA stability and maximize protein expression, (3) a polyadenylation signal, (4) left and right homology arms (LHA and RHA) complementary to the sequences adjoining the predicted site of the Cas9 double-strand break (DSB) in DNA. The donor sequence was encoded with and delivered by a recombinant Adeno-associated virus serotype 6 (AAV6) (FIG. 14C; FIG. 15).

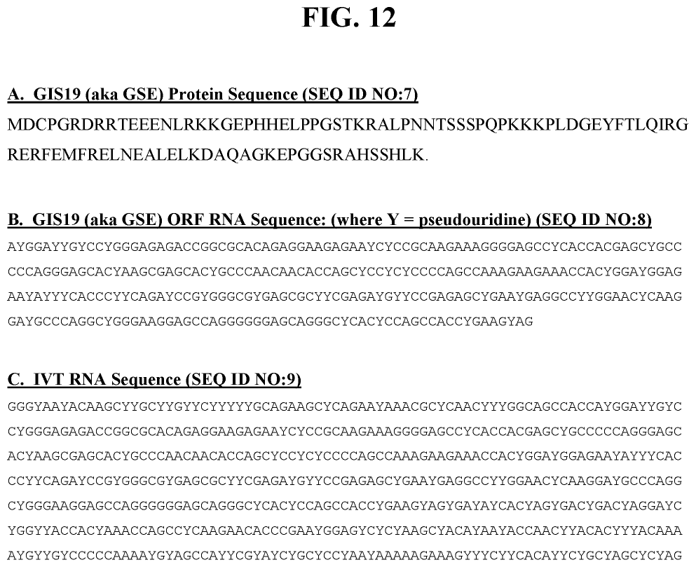

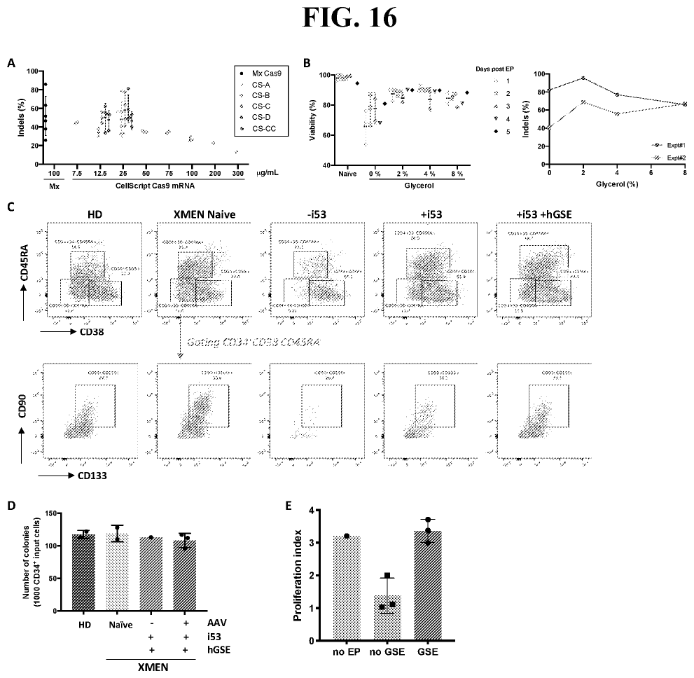

Homology-directed repair of the DSB that is necessary to incorporate the donor sequence is S/G2 phase cell cycle-dependent and competes with the alternate canonical repair pathway by non-homologous end joining (NHEJ) which is rapid, dominant, and active in all cell cycles (20). HDR is therefore rate-limiting for TI in quiescent CD34⁺ HSPCs. After comparing editing efficiency and cell viability following EP with different amounts of Cas9 mRNA and glycerol in CD34⁺ HSPCs (with conditions of 25 μg/mL Cas9 mRNA and 2% glycerol; FIG. 16 A, B), we evaluated the effects of adding mRNA encoding an inhibitor of TP53-binding protein 1 (53BP1) (i53) (21, 22), which is an important factor for NHEJ. With the addition of i53 mRNA, we observed increased AAV-MAGT1 TI rates from 31.45±15.1% (n=3) to 50.0±18.2% (n=9) with i53 (FIG. 14D), confirming the effectiveness of i53 at promoting HDR-mediated TI in HSPCs. We observed that cell viability on day 2 was decreased after gene editing, independently of i53, compared to naive XMEN CD34⁺ HSPCs (FIG. 14E). We hypothesized that the DNA damage response (DDR) to nuclease-induced DSBs caused activation of the p53 pathway and cell cycle arrest, senescence, or apoptosis (23-25). To avoid potential immune responses to rat-derived sequences, we explored the effects of mRNA encoding a humanTP53 inhibitor/GSE CS-56 (“aka GIS19,” FIG. 12) mRNA during EP (FIG. 14D, E). Although TI rates were relatively unchanged (62.3±18%; n=7) (FIG. 14D), the viability of GE CD34⁺ HSPCs was significantly increased with the addition of GSE CS-56 mRNA (FIG. 14E). We observed increased expression of maturation/differentiation markers (CD34⁺CD38⁺) 2 days after GE with AAV compared to untreated XMEN HSPCs (FIG. 14F), although a significant population (30-35%) of primitive CD34⁺CD38-CD45RA-CD90⁺CD133⁺ cells remained (FIG. 16C). A colony forming unit (CFU) assay demonstrated GE CD34⁺ HSPCS retained robust colony formation capabilities (FIG. 16D), and proliferative capacity was better preserved in CD34⁺ HSPCs edited in presence of mRNA-encoded GSE-CS56 (FIG. 16E).

In Vitro Phenotypic and Functional Correction in T and NK Cells Differentiated from Gene-Edited XMEN CD34⁺ Cells

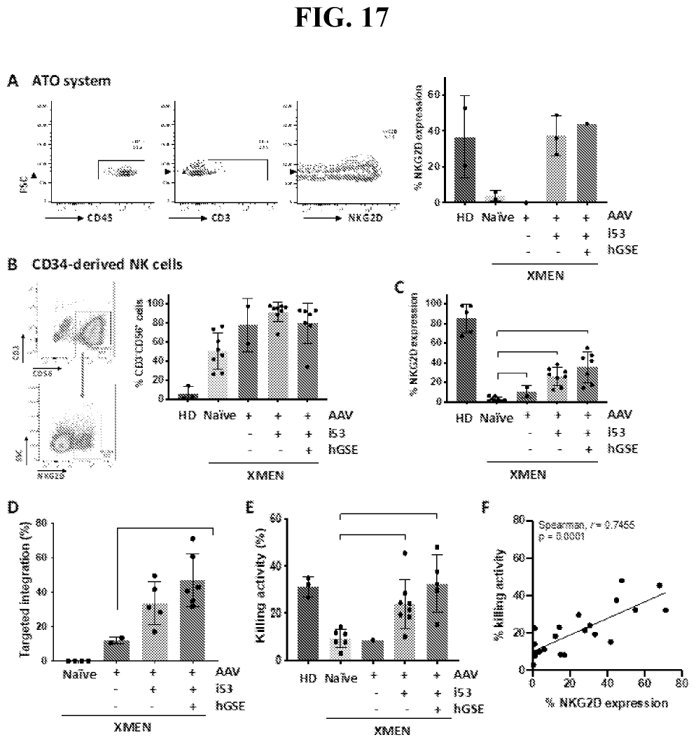

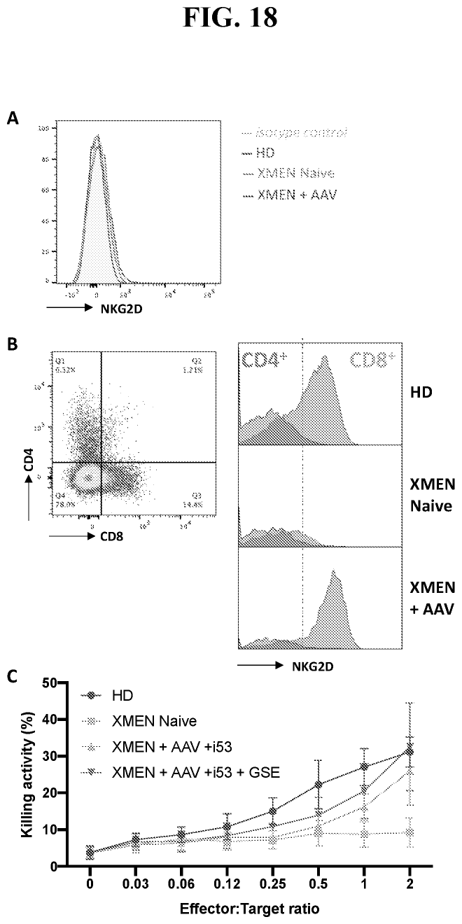

NKG2D receptor is not expressed in CD34⁺ HSPCs (FIG. 17A) so to assess rescue of MAGT1-dependent expression of NKG2D, gene-edited XMEN CD34⁺ HSPCs were differentiated in vitro into T cells using an artificial thymic organoid (ATO) system (26, 27), and into NK cells after coculture with K562-mb15-41BBL (28, 29). After 4-6 weeks coculturing with MS5-DLL4 feeder cells in the ATO system, differentiated CD3⁺ T cells expressed NKG2D at similar levels compared to HD control cells when gene-edited with i53 mRNA, independent of GSE-CS56 mRNA (FIG. 17A). NKG2D expression in gene edited cells was restricted to CD8⁺ cells as in HD control (FIG. 18B). In vitro differentiated NK cells (CD3-CD56⁺) (FIG. 17B) also expressed NKG2D (26% with i53 versus 36% with i53⁺ GSE-CS56) by day 35 of culture (FIG. 17C), with molecular TI rates of 34% and 47% respectively (FIG. 17D).

Next, we assessed cytotoxic function of in vitro differentiated NK cells against the human chronic myelogenous leukemia K562 cell line at different Effector:Target (E:T) ratios (FIG. 18C). Comparable levels between GE XMEN and HD control NK cells were observed at an E:T ratio of 2:1 (FIG. 17E), and killing activity correlated with the level of NKG2D expression (FIG. 17F). Thus, we show TI of MAGT1 in XMEN CD34⁺ HSPCs maintained differentiation capabilities to mature T and NK cells and demonstrated functional MAGT1 correction in restoring physiological expression and function of NKG2D in CD8⁺ T and NK cells.

### Engraftment of Gene-Edited XMEN CD34⁺ Cells and In Vivo Correction

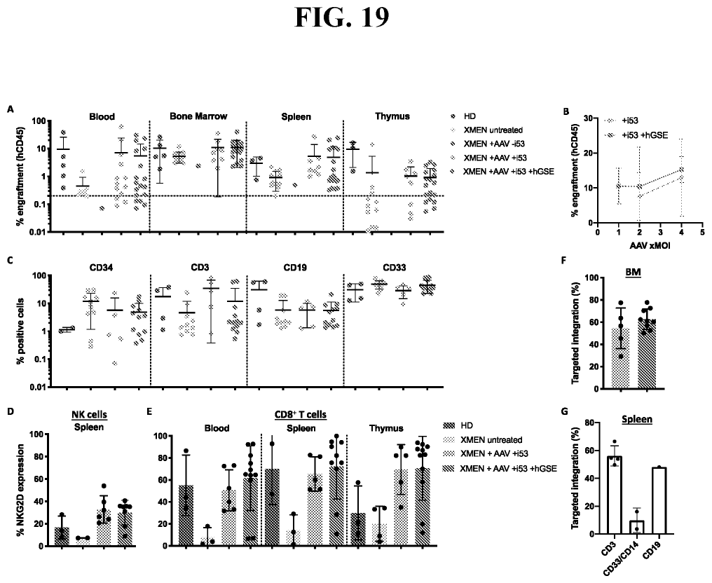

To determine the durability of the improved in vitro gene correction rates, we transplanted XMEN patients' CD34⁺ HSPCs gene edited into irradiated NSGS (NOD.Cg-Prkdc^{scid} Il2rg^{tm1Wjl} Tg(CMV-IL3,CSF2,KITLG)1Eav/MloySzJ) newborn pups via intrahepatic injections (30). At week 16 analysis, we observed robust engraftment of human CD45⁺ cells and colonization of multiple organs (peripheral blood (PB), bone marrow (BM), spleen and thymus) (FIG. 19A) (10.4% for HD, 5.25% for XMEN untreated, 11.03% with i53 and 10.98% with i53 and GSE-CS56 in the BM). Similar engraftment levels were maintained in the BM even at high AAV Multiplicity of Infection (MOIs) (FIG. 19B) contrary to previously reported damaging effects of AAV (18, 19). The relative contribution of myeloid (CD33⁺), B (CD19⁺), and T (CD3⁺) cell percentages in BM was the same as HD controls, indicating maintenance of differentiation capability in GE XMEN HSPCs (FIG. 19C). Interestingly, low levels of human CD34⁺ cells remained detectable in NSGS BM (FIG. 19C).

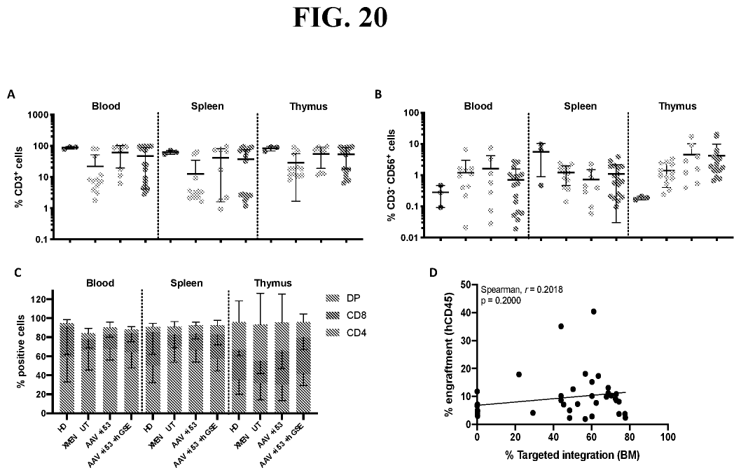

Analysis of PB, spleen and thymus in transplanted NSGS confirmed robust human T cell development with normal percentages of human NK and T cells (FIG. 20 A, B), and normal differentiation to mature CD4⁺, CD8⁺ and double positive (DP) T cells (FIG. 20C). Restoration of NKG2D expression confirmed functional correction of MAGT1 in NK (CD3-CD56⁺) (spleen) and CD8⁺ T cells (PB, spleen, thymus) (FIG. 19D, E). Addition of i53 and GSE-CS56 improved percentages of NKG2D-expressing CD8⁺ T cells (FIG. 19E).

Molecular ddPCR comparing input pre-transplant TI (50.0±18.2% for i53, 62.3±18% for i53⁺ GSE-CS56, FIG. 1E) and post-transplant TI (54.5±18.3% for i53; 62.6±9.2% for i53⁺ GSE-CS56) showed persistence of enhancing effect with i53 and GSE-CS56 (FIG. 19F), indicating efficient targeting of engrafting HSPCs. TI was also confirmed in human myeloid, lymphoid B and T cells from the spleen (FIG. 19G). Higher TI percentages did not impair engraftment despite previous concerns of DNA DSB and AAV triggering DDR known to adversely impact cell survival and engraftment (FIG. 20D).

### Efficient Gene Editing in Peripheral Blood XMEN T Cells

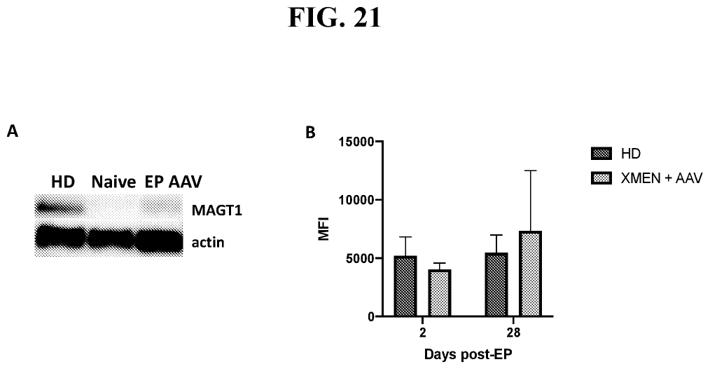

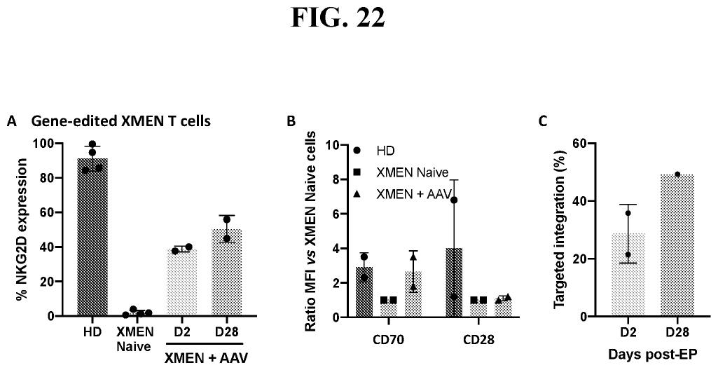

EBV infections and lymphoproliferative disease that occur commonly in XMEN patients are serious medical complications that need prompt treatment. However, human T cell differentiation from engrafted CD34⁺ HSPCs during gene therapy can take months, especially if there is a resident T cell population. To address this potential latency to attaining mature T cell function, we explored correction of PB mature T cells. We employed gene correction of PB XMEN lymphocytes using a CRISPR-Cas9/AAV-MAGT1 approach to enhance the duration of corrected T cells. Pre-stimulated lymphocytes collected by leukapheresis from XMEN patient P1 and GE with i53 mRNA and AAV-MAGT1 showed MAGT1 restoration two days after EP (FIG. 20A). Correction of MAGT1 restored normal expression of NKG2D receptor (FIG. 21A, FIG. 20B) and CD70 (FIG. 21B), but surprisingly, not of CD28 (FIG. 21B). Of note, TI rates almost doubled over 4 weeks' culture (28.6±10.1% at day 2 post-EP, to 49.3% at day 28) (FIG. 21C), as did NKG2D expression (FIG. 21A). The increase in gene correction percentages over time highlight a selective advantage in corrected cells, consistent with the outgrowth of normal cells in PB of XMEN female carriers, limiting mutations detectable only in BM cells (7). The survival advantage of corrected cells reduces the requirement to achieve high rates of gene correction, although more numerous corrected clones remains important to provide diversity to the T-cell repertoire that is important for T cell function.

### Identification and Quantification of Off-Target Events

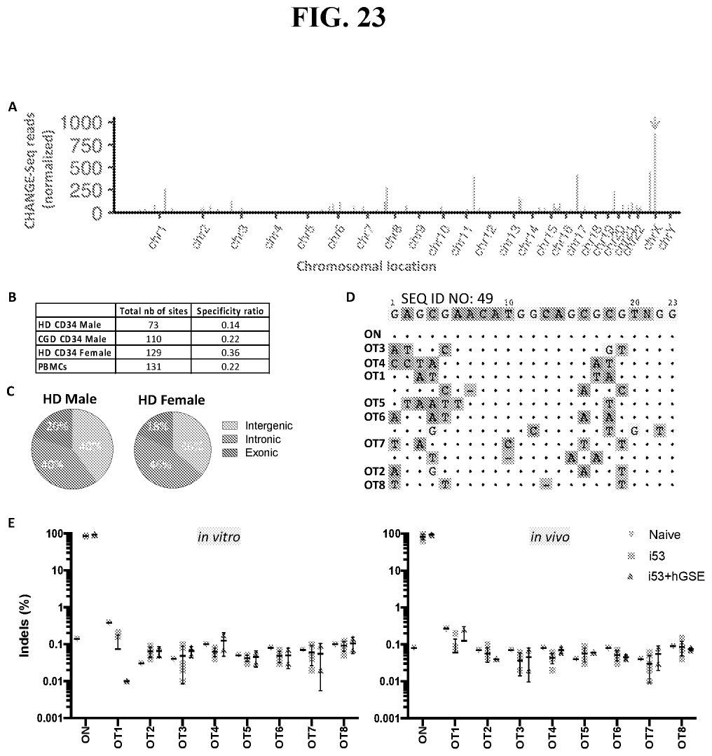

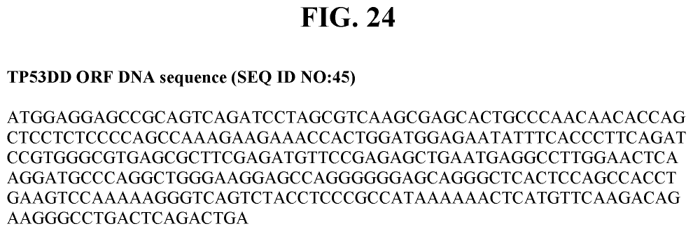

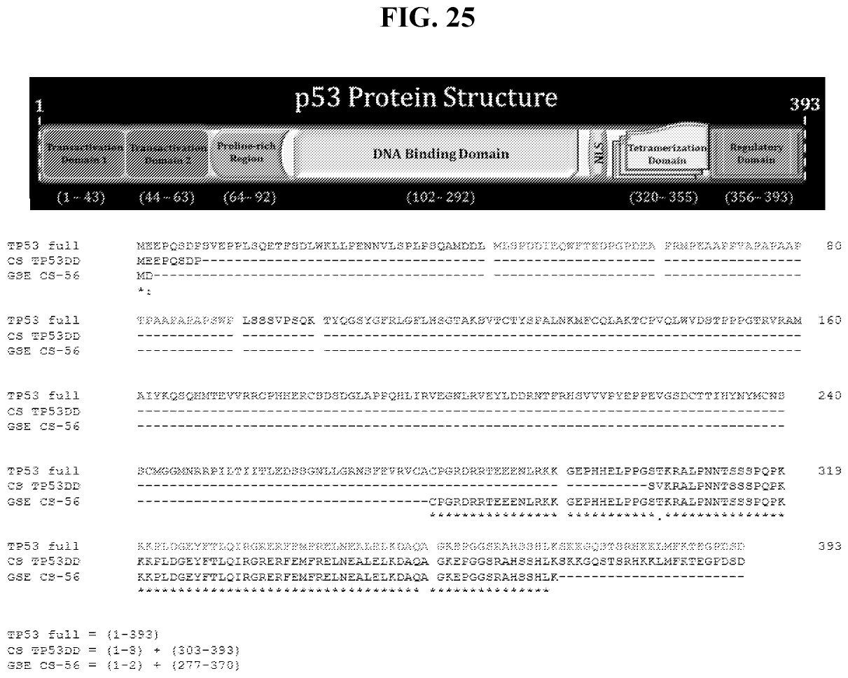

Many in silico computational tools like COSMID (31), Cas-OFFinder (32) and CCTop (33) are available to predict off-target (OT) sites. Here, candidate OT sites were identified using an ultra-sensitive, unbiased, in vitro CHANGE-Seq approach (34) on male and female HD CD34⁺ HSPCs or HD PBMCs. As shown in the Manhattan plot, we observed robust editing of the on-target site and some evidence of in vitro targeting of several OTs (FIG. 23A), with estimated specificity ratios of 0.14 to 0.36 (FIG. 23B). Most of these OTs sites were located in intergenic regions (36-40%) and gene intronic regions (40-46%) while only 14-20% are located in exonic regions of genes or pseudogenes (FIG. 23C). To determine whether we observed editing at the top eight OT candidates in test samples (FIG. 23D and Table S2), targeted next-generation sequencing was performed on unedited and GE XMEN samples (in vitro experiments from CD34⁺ cells and in vivo human cells isolated from BM of transplanted mice at week 16 post-transplant).

TABLE 2

| | | | Predicted by | | | |
|---|---|---|---|---|---|---|
| | Genomic | | Cas- | | | SEQ ID |
| OT | Location (hg38) | Primer sequence (5′→3′) | OFFinder | CCTop | COSMID | NO |
| OT1 | chr11: | OT1-Fwd CGAAGGCCTCTGCTAGG | Yes | Yes | No | 24 and |
| | 111297960-111297983 | OT1-Rev GGGCAGCTAAGAAGCCA | | | | 25 |
| OT2 | chr6: | OT2-Fwd AAAGGCAGTGAGTTGAAGTTT | Yes | Yes | No | 26 and |
| | 92050497-92050520 | OT2-Rev GAGTATGTAAGAACAGCAGGATAAA | | | | 27 |
| OT3 | chrX: | OT3-Fwd CTGTCTCACCCGCACTG | Yes | Yes | No | 28 and |
| | 48911621-48911644 | OT3-Rev AAAGCCACCACCCTGAC | | | | 29 |
| OT4 | chr17: | OT4-Fwd GATCTTGAGGTGTCCCTGATTC | No | No | No | 30 and |
| | 18177476-18177499 | OT4-Rev CTCTGAAGTGCTCTCTGTTGAG | | | | 31 |
| OT5 | chr1: | OT5-Fwd AGCAGGGTGCCTACTTCT | No | No | No | 32 and |
| | 154428671-154428694 | OT5-Rev TCCTCCTCACACTCGTTCTT | | | | 33 |
| OT6 | chr20: | OT6-Fwd AGAAGGTTGAAGTAGTGACTTGT | Yes | Yes | No | 34 and |
| | 7644686-7644709 | OT6-Rev GGAAACTTAACATCTGATCAGCAAA | | | | 35 |
| OT7 | chr13: | OT7-Fwd ATGGCTGTGATAACAAGCAGA | Yes | No | No | 36 and |
| | 108784119-108784142 | OT7-Rev CTTCAGGGATATTCTTCAGTAGACTT | | | | 37 |
| OT8 | chr8: | OT8-Fwd CCTGATTCTTGATTGGCTCTG | No | No | No | 38 and |
| | 17808645-17808667 | OT8-Rev GCCAGCCCATGTTTAGAAG | | | | 39 |

Table 2 provides a list of the eight top off-targets sites. The genomic location and primers used for validation of each OT site are indicated as well as the prediction of this site by in silico computational tools. Compared to unedited XMEN samples, we observed no statistically significant difference in modifications at OTs in our GE treated samples (FIG. 23E), demonstrating that Cas9 using sgRNA #1 is highly specific.

Current therapeutic strategy used clinically to achieve gene correction is an integrating lentivector (LV) to insert a functional cDNA into CD34⁺ HSPCs as demonstrated in primary immunodeficiency diseases such as X-linked Severe Combined Immunodeficiency (35) (Clinical trial NCT 01306019). However, the safety and efficacy of LV-mediated random integration and constitutive expression under the regulation of an exogenous promoter is not known. In that context, CRISPR/Cas9 gene editing targeting the endogenous locus represents a promising approach as a universal treatment for patients presenting with XMEN disease. We demonstrate here for the first time an efficient and durable gene correction of CD34⁺ HSPCs and peripheral blood T cells from XMEN patients.

Gene editing technology is a continuously improving field (36), and among the different strategies investigated in recent years to improve the HDR-mediated repair in HSPCs (19, 37, 38), the most promising is the inhibition of the 53BP1 protein recruited during the NHEJ repair (21). Indeed, using i53 mRNA during the gene-editing process, researchers are now routinely reaching levels of HDR-mediated TI with AAV donors or HDR-mediated gene correction with ssODN donors above 60% in HSPCs from patients presenting with various diseases like CGD (Sweeney 2020; SDR 2020), SCD (Tisdale 2020) and SCID-X1, and this was shown here in the context of XMEN disease.

In this Example, we chose to use an AAV donor that can carry an entire cDNA in order to potentially correct any kind of mutation in MAGT1 gene. However, one main limit to the use of AAV is the reduced engraftment capacity of gene-edited HSCs and lower level of correction in vivo compared to in vitro input cells observed by us and others (18, 19). Indeed, the gene editing process induces the activation of p53 pathway thereby triggering cell-cycle arrest and apoptosis (23-25). We showed here the efficiency of GSE-CS56 mRNA (which inhibits TP53) to rescue the cell viability of edited HSPCs. However, surprisingly, our mice transplantation studies showed that human engraftment was similar whatever the presence of GSE-CS56, and high targeted efficiency was maintained in vivo after 16 weeks in both conditions demonstrating that engraftable HSPCs were corrected. To note, although not significant, TI levels and NKG2D expression in CD8⁺ T cells were slightly improved in presence of GSE-CS56 mRNA.

Whether the use of the enhancers during the gene-editing process induces a safety risk is not known. In order to minimize as much as possible the impact of the gene-editing process, we used the mRNA encoding for Cas9 and enhancers to reduce the exposure of the cells to these components. The objective of the use of Cas9 mRNA and thus transient expression of Cas9 protein is also to decrease the off-target activity (39) although some recent studies have described that electroporation of Cas9 mRNA or IVT gRNA modifies transcriptional expression of immune response genes (activation of type I interferon pathway) (40, 41). Generation of off-targets remain the main concern when using gene editing approach, especially when the purpose is for therapeutic use in patients' cells. We performed off-targets analysis and showed very low OT activity at the 8 identified OT sites. Identification of off-targets sites using in vivo genome-wide off-target analysis versus in silico computational analysis prediction can give very different results. Comparing the CHANGE-seq with in silico prediction tools Cas-OFFinder, CCTop and COSMID, we found that Cas-OFFinder was the most reliable with the prediction of 5 of the 8 top sites (62.5%) identified with CHANGE-seq (Table 2).

EBV-associated lymphomas remain the main cause of severe morbidity, affecting ˜65% of XMEN patients. Adoptive transfer of allogenic virus-specific T cells is an alternative option, but it can potentially induce anti-HLA alloimmunity of the receiver and complicate future HSC transplantation. CRISPR/Cas9 gene editing could provide a long-lasting correction of patients' T cells and a more sustainable protection of the patient after infusion of autologous gene-edited T cells. Here, we showed good targeted integration in XMEN T cells at the molecular level (20-40%), lower than in CD34⁺ cells, but comparable to other studies (42-44). This level of genomic correction in edited T cells was sufficient to restore MAGT1 expression and function in N-glycosylation as suggested by the increased expression of the N-glycoproteins NKG2D and CD70. As expected, restoration of the downstream NKG2D receptor expression was restricted to CD8⁺ T cells and with the same level of expression than HD cells. In addition, we observed a survival advantage in corrected T cells in culture, which improves likelihood of clinical benefit from even low numbers of corrected cells. The safety of CRISPR/Cas9 approach for T cell after infusion of edited cells is currently demonstrated in the context of CAR-T cells in a phase I trial for the treatment of refractory cancer (45).

In conclusion, functional gene correction with GE enhancers of CD34⁺ HSPCs and PB T cells offers a two-pronged approach for more immediate protection in patients with acute infections, while latent T cell differentiation from engrafted GE CD34⁺ HSPCs for permanent correction. The GE enhancers are widely applicable for improving the levels of engrafting GE HSPCs for HDR-mediated gene correction for treatment of many blood and immune cell diseases.

### REFERENCES

1. Blommaert, et al., Mutations in MAGT1 lead to a glycosylation disorder with a variable phenotype. P Natl Acad Sci USA 116, 9865-9870 (2019).
2. M. Matsuda-Lennikov, M. Biancalana, J. Zou, J. C. Ravell, L. Zheng, C. Kanellopoulou, P. Jiang, G. Notarangelo, H. Jing, E. Masutani, A. J. Oler, L. R. Olano, B. L. Schulz, M. J. Lenardo, Magnesium transporter 1 (MAGT1) deficiency causes selective defects in N-linked glycosylation and expression of immune-response genes. J Biol Chem 294, 13638-13656 (2019).
3. J. C. Ravell, M. Matsuda-Lennikov, S. D. Chauvin, J. Zou, M. Biancalana, S. J. Deeb, S. Price, H. C. Su, G. Notarangelo, P. Jiang, A. Morawski, C. Kanellopoulou, K. Binder, R. Mukherjee, J. T. Anibal, B. Sellers, L. Zheng, T. He, A. B. George, S. Pittaluga, A. Powers, D. E. Kleiner, D. Kapuria, M. Ghany, S. Hunsberger, J. I. Cohen, G. Uzel, J. Bergerson, L. Wolfe, C. Toro, W. Gahl, L. R. Folio, H. Matthews, P. Angelus, I. K. Chinn, J. S. Orange, C. M. Trujillo-Vargas, J. L. Franco, J. Orrego-Arango, S. Gutierrez-Hincapie, N. C. Patel, K. Raymond, T. Patiroglu, E. Unal, M. Karakukcu, A. G. Day, P. Mehta, E. Masutani, S. S. De Ravin, H. L. Malech, G. Altan-Bonnet, V. K. Rao, M. Mann, M. J. Lenardo, Defective glycosylation and multisystem abnormalities characterize the primary immunodeficiency XMEN disease. J Clin Invest 130, 507-522 (2020).
4. F. Y. Li, B. Chaigne-Delalande, H. L. Su, G. Uzel, H. Matthews, M. J. Lenardo, XMEN disease: a new primary immunodeficiency affecting Mg2⁺ regulation of immunity against Epstein-Barr virus. Blood 123, 2148-2152 (2014).
5. J. Ravell, B. Chaigne-Delalande, M. Lenardo, X-linked immunodeficiency with magnesium defect, Epstein-Barr virus infection, and neoplasia disease: a combined immune deficiency with magnesium defect. Curr Opin Pediatr 26, 713-719 (2014).
6. F. Y. Li, M. J. Lenardo, B. Chaigne-Delalande, Loss of MAGT1 abrogates the Mg2+ flux required for T cell signaling and leads to a novel human primary immunodeficiency.

Magnesium Res 24, S109-S114 (2011).
7. F. Y. Li, B. Chaigne-Delalande, C. Kanellopoulou, J. C. Davis, H. F. Matthews, D. C. Douek, J. I. Cohen, G. Uzel, H. C. Su, M. J. Lenardo, Second messenger role for Mg2+ revealed by human T-cell immunodeficiency. Nature 475, 471-476 (2011).
8. F. Y. Li, B. Chaigne-Delalande, G. O'Connor, M. Lukacs, L. X. Zheng, A. Shatzer, H. Matthews, H. Su, J. Cohen, G. Uzel, M. Lenardo, Intracellular free Mg2⁺ is required to maintain NKG2D expression necessary for controlling EBV infection in XMEN disease. J Immunol 190, (2013).
9. J. D. Coudert, W. Held, The role of the NKG2D receptor for tumor immunity. Semin Cancer Biol 16, 333-343 (2006).
10. B. Chaigne-Delalande, F. Y. Li, G. M. O'Connor, M. J. Lukacs, P. Jiang, L. X. Zheng, A. Shatzer, M. Biancalana, S. Pittaluga, H. F. Matthews, T. J. Jancel, J. J. Bleesing, R. A. Marsh, T. W. Kuijpers, K. E. Nichols, C. L. Lucas, S. Nagpal, H. Mehmet, H. C. Su, J. I. Cohen, G. Uzel, M. J. Lenardo, Mg2+ Regulates Cytotoxic Functions of NK and CD8 T Cells in Chronic EBV Infection Through NKG2D. Science 341, 186-191 (2013).
11. H. H. Akar, T. Patiroglu, M. Hershfield, M. van der Burg, Combined immunodeficiencies: twenty years experience from a single center in Turkey. Cent Eur J Immunol 41, 107-115 (2016).
12. D. Dimitrova, J. J. Rose, G. Uzel, J. I. Cohen, K. V. Rao, J. H. Bleesing, C. G. Kanakry, J. A. Kanakry, Successful Bone Marrow Transplantation for XMEN: Hemorrhagic Risk Uncovered. J Clin Immunol 39, 1-3 (2019).
13. E. M. Klinken, P. E. Gray, B. Pillay, L. Worley, E. S. J. Edwards, K. Payne, B. Bennetts, D. Hung, B. A. Wood, J. J. Chan, G. M. Marshall, R. Mitchell, G. Uzel, C. S. Ma, S. G. Tangye, A. McLean-Tooke, Diversity of XMEN Disease: Description of 2 Novel Variants and Analysis of the Lymphocyte Phenotype. J Clin Immunol 40, 299-309 (2020).
14. D. B. Kohn, C. Y. Kuo, New frontiers in the therapy of primary immunodeficiency: From gene addition to gene editing. J Allergy Clin Immunol 139, 726-732 (2017).
15. Z. Y. Zhang, A. J. Thrasher, F. Zhang, Gene therapy and genome editing for primary immunodeficiency diseases. Genes Dis 7, 38-51 (2020).
16. C. Booth, R. Romano, M. G. Roncarolo, A. J. Thrasher, Gene therapy for primary immunodeficiency. Hum Mol Genet 28, R15-R23 (2019).
17. S. S. De Ravin, J. Brault, CRISPR/Cas9 applications in gene therapy for primary immunodeficiency diseases. Emerging Topics in Life Sciences 3, 277-287 (2019).
18. Z. Romero, A. Lomova, S. Said, A. Miggelbrink, C. Y. Kuo, B. Campo-Fernandez, M. D. Hoban, K. E. Masiuk, D. N. Clark, J. Long, J. M. Sanchez, M. Velez, E. Miyahira, R. Zhang, D. Brown, X. Wang, Y. Z. Kurmangaliyev, R. P. Hollis, D. B. Kohn, Editing the Sickle Cell Disease Mutation in Human Hematopoietic Stem Cells: Comparison of Endonucleases and Homologous Donor Templates. Mol Ther 27, 1389-1406 (2019).
19. A. Lomova, D. N. Clark, B. Campo-Fernandez, C. Flores-Bjurstrom, M. L. Kaufman, S. Fitz-Gibbon, X. Wang, E. Y. Miyahira, D. Brown, M. A. DeWitt, J. E. Corn, R. P. Hollis, Z. Romero, D. B. Kohn, Improving Gene Editing Outcomes in Human Hematopoietic Stem and Progenitor Cells by Temporal Control of DNA Repair. Stem Cells 37, 284-294 (2019).
20. Z. Mao, M. Bozzella, A. Seluanov, V. Gorbunova, DNA repair by nonhomologous end joining and homologous recombination during cell cycle in human cells. Cell Cycle 7, 2902-2906 (2008).
21. M. D. Canny, N. Moatti, L. C. K. Wan, A. Fradet-Turcotte, D. Krasner, P. A. Mateos-Gomez, M. Zimmermann, A. Orthwein, Y. C. Juang, W. Zhang, S. M. Noordermeer, E. Seclen, M. D. Wilson, A. Vorobyov, M. Munro, A. Ernst, T. F. Ng, T. Cho, P. M. Cannon, S. S. Sidhu, F. Sicheri, D. Durocher, Inhibition of 53BP1 favors homology-dependent DNA repair and increases CRISPR-Cas9 genome-editing efficiency. Nat Biotechnol 36, 95-102 (2018).
22. T. S. Nambiar, P. Billon, G. Diedenhofen, S. B. Hayward, A. Taglialatela, K. H. Cai, J. W. Huang, G. Leuzzi, R. Cuella-Martin, A. Palacios, A. Gupta, D. Egli, A. Ciccia, Stimulation of CRISPR-mediated homology-directed repair by an engineered RAD18 variant. Nature Communications 10, (2019).
23. G. Schiroli, A. Conti, S. Ferrari, L. Della Volpe, A. Jacob, L. Albano, S. Beretta, A. Calabria, V. Vavassori, P. Gasparini, E. Salataj, D. Ndiaye-Lobry, C. Brombin, J. Chaumeil, E. Montini, I. Merelli, P. Genovese, L. Naldini, R. Di Micco, Precise Gene Editing Preserves Hematopoietic Stem Cell Function following Transient p53-Mediated DNA Damage Response. Cell Stem Cell 24, 551-565 e558 (2019).
24. E. Haapaniemi, S. Botla, J. Persson, B. Schmierer, J. Taipale, CRISPR-Cas9 genome editing induces a p53-mediated DNA damage response. Nature Medicine 24, 927−+(2018).
25. R. J. Ihry, K. A. Worringer, M. R. Salick, E. Frias, D. Ho, K. Theriault, S. Kommineni, J. Chen, M. Sondey, C. Y. Ye, R. Randhawa, T. Kulkarni, Z. Yang, G. McAllister, C. Russ, J. Reece-Hoyes, W. Forrester, G. R. Hoffman, R. Dolmetsch, A. Kaykas, p53 inhibits CRISPR-Cas9 engineering in human pluripotent stem cells. Nature Medicine 24, 939−+(2018).
26. C. S. Seet, C. B. He, M. T. Bethune, S. W. Lie, B. Chick, E. H. Gschweng, Y. H. Zhu, K. Kim, D. B. Kohn, D. Baltimore, G. M. Crooks, A. Montel-Hagen, Generation of mature T cells from human hematopoietic stem and progenitor cells in artificial thymic organoids. Nature Methods 14, 521−+(2017).
27. M. Bosticardo, F. Pala, E. Calzoni, O. M. Delmonte, K. Dobbs, C. L. Gardner, N. Sacchetti, T. Kawai, E. K. Garabedian, D. Draper, J. R. E. Bergerson, S. S. DeRavin, A. F. Freeman, T. Gungor, N. Hartog, S. M. Holland, D. B. Kohn, H. L. Malech, M. L. Markert, K. G. Weinacht, A. Villa, C. S. Seet, A. Montel-Hagen, G. M. Crooks, L. D. Notarangelo, Artificial thymic organoids represent a reliable tool to study T-cell differentiation in patients with severe T-cell lymphopenia. Blood Adv 4, 2611-2616 (2020).
28. H. Fujisaki, H. Kakuda, N. Shimasaki, C. Imai, J. Ma, T. Lockey, P. Eldridge, W. H. Leung, D. Campana, Expansion of highly cytotoxic human natural killer cells for cancer cell therapy. Cancer Res 69, 4010-4017 (2009).
29. T. Kamiya, Y. H. Chang, D. Campana, Expanded and Activated Natural Killer Cells for Immunotherapy of Hepatocellular Carcinoma. Cancer Immunol Res 4, 574-581 (2016).
30. M. Wunderlich, F. S. Chou, K. A. Link, B. Mizukawa, R. L. Perry, M. Carroll, J. C. Mulloy, AML xenograft efficiency is significantly improved in NOD/SCID-IL2RG mice constitutively expressing human SCF, GM-CSF and IL-3. Leukemia 24, 1785-1788 (2010).
31. T. J. Cradick, P. Qiu, C. M. Lee, E. J. Fine, G. Bao, COSMID: A Web-based Tool for Identifying and Validating CRISPR/Cas Off-target Sites. Mol Ther Nucleic Acids 3, e214 (2014).
32. S. Bae, J. Park, J. S. Kim, Cas-OFFinder: a fast and versatile algorithm that searches for potential off-target sites of Cas9 RNA-guided endonucleases. Bioinformatics 30, 1473-1475 (2014).
33. M. Stemmer, T. Thumberger, M. D. Keyer, J. Wittbrodt, J. L. Mateo, CCTop: An Intuitive, Flexible and Reliable CRISPR/Cas9 Target Prediction Tool. Plos One 10, (2015).
34. C. R. Lazzarotto, N. L. Malinin, Y. Li, R. Zhang, Y. Yang, G. Lee, E. Cowley, Y. He, X. Lan, K. Jividen, V. Katta, N. G. Kolmakova, C. T. Petersen, Q. Qi, E. Strelcov, S. Maragh, G. Krenciute, J. Ma, Y. Cheng, S. Q. Tsai, CHANGE-seq reveals genetic and epigenetic effects on CRISPR-Cas9 genome-wide activity. Nat Biotechnol, (2020).
35. S. S. De Ravin, X. Wu, S. Moir, S. Anaya-O'Brien, N. Kwatemaa, P. Littel, N. Theobald, U. Choi, L. Su, M. Marquesen, D. Hilligoss, J. Lee, C. M. Buckner, K. A. Zarember, G. O'Connor, D. McVicar, D. Kuhns, R. E. Throm, S. Zhou, L. D. Notarangelo, I. C. Hanson, M. J. Cowan, E. Kang, C. Hadigan, M. Meagher, J. T. Gray, B. P. Sorrentino, H. L. Malech, L. Kardava, Lentiviral hematopoietic stem cell gene therapy for X-linked severe combined immunodeficiency. Sci Transl Med 8, 335ra357 (2016).
36. M. Broeders, P. Herrero-Hernandez, M. P. T. Ernst, A. T. van der Ploeg, W. Pijnappel, Sharpening the Molecular Scissors: Advances in Gene-Editing Technology. iScience 23, 100789 (2020).
37. C. T. Charlesworth, J. Camarena, M. K. Cromer, S. Vaidyanathan, R. O. Bak, J. M. Carte, J. Potter, D. P. Dever, M. H. Porteus, Priming Human Repopulating Hematopoietic Stem and Progenitor Cells for Cas9/sgRNA Gene Targeting. Mol Ther Nucleic Acids 12, 89-104 (2018).
38. Q. Guo, G. Mintier, M. Ma-Edmonds, D. Storton, X. Wang, X. Xiao, B. Kienzle, D. Zhao, J. N. Feder, ‘Cold shock’ increases the frequency of homology directed repair gene editing in induced pluripotent stem cells. Sci Rep 8, 2080 (2018).
39. H. X. Zhang, Y. Zhang, H. Yin, Genome Editing with mRNA Encoding ZFN, TALEN, and Cas9. Mol Ther 27, 735-746 (2019).
40. M. K. Cromer, S. Vaidyanathan, D. E. Ryan, B. Curry, A. B. Lucas, J. Camarena, M. Kaushik, S. R. Hay, R. M. Martin, I. Steinfeld, R. O. Bak, D. P. Dever, A. Hendel, L. Bruhn, M. H. Porteus, Global Transcriptional Response to CRISPR/Cas9-AAV6-Based Genome Editing in CD34(+) Hematopoietic Stem and Progenitor Cells. Mol Ther 26, 2431-2442 (2018).
41. S. Kim, T. Koo, H. G. Jee, H. Y. Cho, G. Lee, D. G. Lim, H. S. Shin, J. S. Kim, CRISPR RNAs trigger innate immune responses in human cells. Genome Res, (2018).
42. M. Goodwin, E. Lee, U. Lakshmanan, S. Shipp, L. Froessl, F. Barzaghi, L. Passerini, M. Narula, A. Sheikali, C. M. Lee, G. Bao, C. S. Bauer, H. K. Miller, M. Garcia-Lloret, M. J. Butte, A. Bertaina, A. Shah, M. Pavel-Dinu, A. Hendel, M. Porteus, M. G. Roncarolo, R. Bacchetta, CRISPR-based gene editing enables FOXP3 gene repair in IPEX patient cells. Sci Adv 6, eaaz0571 (2020).
43. C. Y. Kuo, J. D. Long, B. Campo-Fernandez, S. de Oliveira, A. R. Cooper, Z. Romero, M. D. Hoban, A. V. Joglekar, G. R. Lill, M. L. Kaufman, S. Fitz-Gibbon, X. Wang, R. P. Hollis, D. B. Kohn, Site-Specific Gene Editing of Human Hematopoietic Stem Cells for X-Linked Hyper-IgM Syndrome. Cell Rep 23, 2606-2616 (2018).
44. J. Eyquem, J. Mansilla-Soto, T. Giavridis, S. J. van der Stegen, M. Hamieh, K. M. Cunanan, A. Odak, M. Gonen, M. Sadelain, Targeting a CAR to the TRAC locus with CRISPR/Cas9 enhances tumour rejection. Nature 543, 113-117 (2017).
45. E. A. Stadtmauer, J. A. Fraietta, M. M. Davis, A. D. Cohen, K. L. Weber, E. Lancaster, P. A. Mangan, I. Kulikovskaya, M. Gupta, F. Chen, L. Tian, V. E. Gonzalez, J. Xu, I. Y. Jung, J. J. Melenhorst, G. Plesa, J. Shea, T. Matlawski, A. Cervini, A. L. Gaymon, S. Desjardins, A. Lamontagne, J. Salas-Mckee, A. Fesnak, D. L. Siegel, B. L. Levine, J. K. Jadlowsky, R. M. Young, A. Chew, W. T. Hwang, E. O. Hexner, B. M. Carreno, C. L. Nobles, F. D. Bushman, K. R. Parker, Y. Qi, A. T. Satpathy, H. Y. Chang, Y. Zhao, S. F. Lacey, C. H. June, CRISPR-engineered T cells in patients with refractory cancer. Science 367, (2020).
46. F. Y. Li, B. Chaigne-Delalande, H. Su, G. Uzel, H. Matthews, M. J. Lenardo, XMEN disease: a new primary immunodeficiency affecting Mg2⁺ regulation of immunity against Epstein-Barr virus. Blood 123, 2148-2152 (2014).
47. Y. Li, D. L. Hermanson, B. S. Moriarity, D. S. Kaufman, Human iPSC-derived natural killer cells engineered with chimeric antigen receptors enhance anti-tumor activity. Cell Stem Cell 23, 181-+(2018).
48. E. K. Brinkman, B. van Steensel, Rapid Quantitative Evaluation of CRISPR Genome Editing by TIDE and TIDER. Methods Mol Biol 1961, 29-44 (2019).
49. E. K. Brinkman, T. Chen, M. Amendola, B. van Steensel, Easy quantitative assessment of genome editing by sequence trace decomposition. Nucleic Acids Res 42, e168 (2014).
50. F. Alkan, A. Wenzel, C. Anthon, J. H. Havgaard, J. Gorodkin, CRISPR-Cas9 off-targeting assessment with nucleic acid duplex energy parameters. Genome Biol 19, 177 (2018).
51. PCT application WO2021016299
52. U.S. Pat. Pub. 20210000926
53. V. Calvanese et al, MLLT3 governs human haematopoietic stem-cell self-renewal and engraftment, Nature 576(7786), 281-286, 2019.
54. Diane Yang et al, Enrichment of G2/M cell cycle phase in human pluripotent stem cells enhances HDR-mediated gene repair with customizable endonucleases, Scientific Reports 6, 21264 2016.
55. Yuxuan Wu et al, Highly efficient therapeutic gene editing of human hematopoietic stem cells, Nat Med 25(5), 776-783, 2019 May.
56. N Raj and LD Attardi, The transactivation domains of the p53 protein, Cold Spring Harb Perspect Med. 2017 January; 7(1), a026047.
57. R Sugimura et al, Haematopoietic stem and progenitor cells from human pluripotent stem cells, Nature 545(7655), 432-438, 2017.
58. A Fradet-Turcotte et al, 53BP1 is a reader of the DNA damage-induced H2A Lys15 ubiquitin mark, Nature 499(7456), 50-54, 2013
59. Jeanette Rientjes, Jieqiong Lou, Menachem Gunzburg, Bradley Doak, Jacqueline L. Norris, Caroline Foley, Sarah Dishman, Stephanie Cholensky, Dirk Truman, Emily Kelly, Susan Thomas, Michelle Meilak, Alyce Lynas, Bec Hurley, Fleur Rodda, Cassey Grist, Emma Rooney, Tanya Lennane, Leanne Hawkey, Ian Smyth, Kenneth Pearce, Elizabeth Hinde, Lindsey James-Ingerman and Stephen Headey, A 53BP1 inhibitory compound enhances CRISPR efficiency for generating knock-in mice, SSRN Electronic Journal (2020)

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A composition or system comprising:
a) a human or animal cell having a mutation in its genomic DNA that is responsible for a disease or condition in a subject from which said cell was derived;
b) a first mRNA encoding a first protein, wherein said first protein is capable of reducing, suppressing or preventing an innate immune response, and
c) a second mRNA encoding a second protein that inhibits 53BPI,
d) a third mRNA encoding a third protein comprising a Cas endonuclease for use in CRISPR DNA editing by homology directed repair, and
e) guide RNA comprising a tracrRNA having a sequence that binds to said Cas nuclease and a CRISPR RNA (crRNA) having a short “guide” sequence that is complementary to and binds to a specific target cleavage site in the genomic DNA that is near to said mutation.

2. The composition or system of claim 1, wherein said guide RNA comprises tracrRNA and crRNA as separate molecules, (collectively “gRNA”) or joined together to form single guide RNA (sgRNA).

3. The composition or system of claim 1, further comprising: a donor oligodeoxynucleotide (donor ODN) or donor DNA template that has a central region that is complementary to the desired corrected sequence for said mutation region in said genomic DNA, and flanking regions on each side of the central region that are complementary to and abut the sequences on each side of the desired corrected sequence for said mutation, wherein said donor oligodeoxynucleotide (donor ODN) or donor DNA template is configured to replace said disease/condition-causing mutation region in said genomic DNA by homology-directed repair (HDR) after said genomic DNA is cleaved by said CRISPR-Cas nuclease at said target cleavage site.

4. The composition or system of claim 1, wherein said first protein is a human mutant p53 tumor suppressor protein (mutant TP53), wherein said mutant TP53 protein lacks all or substantially all of the transactivation domains, the proline-rich region, and the DNA binding domain, but comprises all or substantially all of each of the tetramerization domain and the regulatory domain, thus enabling oligomerization of said mutant TP53 with wild-type TP53 proteins in said cell to form defective tetrameric proteins that results in a dominant negative inhibitory effect on said TP53 activity in said cell.

5. The system or composition of claim 1, wherein said first protein is GSE CS-56 or CS TP53DD.

6. The composition or system of claim 1, further comprising at least one of the following:
(g-i) glycerol, wherein the final percent concentration of said glycerol in said composition or system is selected from: 2%, 3%, 4%, 5%, 6%, 7%, 8%, 9% and 10%,
wherein said glycerol results in at least one of the following benefits:
1) increased viability of said cells during electroporation;
2) increased rates of survival of said gene-edited cells that have on-target corrections of said mutation due said gene editing; and/or
3) increased rates of transplantation of HSPCs said gene-edited cells that have on-target corrections of said mutation due said gene editing in vivo; or
(g-ii) trimethylglycine (aka glycine betaine or betaine), wherein the final percent concentration of said glycine betaine in said composition or system is selected from:
1-5%, 5-10%, 10-15%, 15-20%, and 20-25%,
wherein said trimethylglycine results in at least one of the following benefits:
1) increased viability of said cells during electroporation;
2) increased rates of survival of said gene-edited cells that have on-target corrections of said mutation due said gene editing; and/or
3) increased rates of transplantation of HSPCs said gene-edited cells that have on-target corrections of said mutation due said gene editing in vivo; or
(g-iii) dimethylsulfoxide DMSO), wherein the final percent concentration of said DMSO in said composition or system is selected from: 0.1-1%, 1%-5%, 5-10%, 10-15%, 15-20%, 20-25%, 25-30%; and 30-35%;
wherein said DMSO results in at least one of the following benefits:
1) increased viability of said cells during electroporation;
2) increased rates of survival of said gene-edited cells that have on-target corrections of said mutation due said gene editing; and/or
3) increased rates of transplantation of HSPCs said gene-edited cells that have on-target corrections of said mutation due said gene editing in vivo.

7. The composition or system of claim 1, further comprising:
h) a cell cycle modulator that inhibits microtubule polymerization, arresting cells in the G2/M phase of the cell cycle, selected from the group consisting of ABT−751, and Nocodazole.

8. The composition or system of any of claim 1, wherein said in vitro-synthesized modified mRNA molecules are purified using a process that removes RNA contaminant molecules that are immunogenic and toxic to the cell by inducing an innate immune response, as can be detected by measuring decreased secretion of IL-12, INF-alpha or TNF-alpha cytokine from monocyte-derived dendritic cells (MDDCs) transfected with said purified modified mRNA molecules compared to secretion of said cytokine from MDDCs transfected with the unpurified modified mRNA molecules, such that said purified RNA preparation is free of RNA contaminant molecules that, if present, would activate an immune response in said cell sufficient to prevent survival of said cell.

9. The composition or system of claim 1, wherein said cells are human cells and comprise at least one of the following: a hematopoietic stem cell (HSC), a hemopoietic progenitor cell (HPC), an induced pluripotent stem cell (iPSC), a mixture of HSCs and HPCs (HSPCs), a T cell, or a cord blood HSC.

10. The composition or system of any of claim 1, wherein said first protein comprises: a p53 protein (TP53) inhibitor.

11. The composition or system of claim 5, wherein said TP53 inhibitor comprises: i) a TP53 variant protein comprising one or more mutations that inhibit wild-type TP53 expression, ii) pifithrin-alpha, iii) Pifithrin-α hydrobromide; and iv) Cyclic Pifithrin-α hydrobromide.

12. The composition or system of claim 6, wherein said TP53 variant protein is selected from the group consisting of: GSE56, GSE-CS-19, GSE CS-56, TP53DD.

13-18. (canceled)

19. The composition or system of any of claim 1, wherein said mRNAs encoding said first, second, and third proteins comprise at least one modified nucleoside selected from the group consisting of: pseudouridine (Ψ), 1-methylpseudouridine (m¹Ψ), 5-methyluridine (m⁵U), 5-methoxyuridine (mo⁵U), 2′-O-methyluridine (Um or m^{2′-O}U), and 2-thiouridine (s²U) in place of uridine, wherein said modified mRNAs comprising said modified nucleoside are significantly less immunogenic than the counterpart mRNA that does not comprise said modified nucleoside in place of uridine; and 5-methylcytidine (m⁵C) in place unmodified nucleoside cytidine; or N⁶-methyladenosine (m⁶A) in place of unmodified adenosine.

20. The composition or system claim 19, wherein all or most of the uridines in said first, second, and third mRNAs are replaced with pseudouridine (Ψ), 1-methylpseudouridine (m¹Ψ), 5-methyluridine (m⁵U), 5-methoxyuridine (mo⁵U).

21. The composition or system of claim 19, wherein all or most of the cytidines in said first, second, or third mRNA are replaced with 5-methylcytidine (m⁵C).

22-31. (canceled)

32. A method of generating a gene-edited stem or progenitor cell comprising:
a) contacting an initial cell from a subject with first, second, third, fourth, and fifth reagents in vitro,
wherein said initial cell comprises a mutation in its genome that is responsible for a disease or condition in a subject from which cell was derived,
wherein said first reagent comprises a first mRNA encoding a first protein, wherein said first protein is capable of reducing, suppressing or preventing an innate immune response in said initial stem or progenitor cell,
wherein said second reagent comprises a second mRNA encoding a second protein that inhibits 53BPI,
wherein said third reagent comprises a third mRNA encoding a third protein, wherein
said third protein comprises a Cas nuclease for use in CRISPR,
wherein said fourth reagent comprises a guide RNA (gRNA or sgRNA) comprising a tracrRNA having a sequence that binds to a Cas enzyme and a CRISPR RNA (crRNA) having a short “guide” sequence that is complementary to and binds to a specific target cleavage site in the genome that is near to said mutation, and
wherein said fifth reagent comprises a donor oligodeoxynucleotide (donor ODN), wherein said ODN comprises two outer flanking regions that are complementary to one strand of the genomic DNA on each side of the mutation site region in said genomic DNA and a middle region of interest, wherein said region of interest is configured to replace said disease/condition-causing mutation region in said genomic DNA by homology-directed repair (HDR) after said genomic DNA is cleaved by said CRISPR-Cas nuclease at said target cleavage site,
wherein said contacting is under conditions such that said region of interest replaces said disease/condition-causing mutation region in said genomic DNA, thereby generating a gene-edited cell.

33. The method of claim 32, wherein said contacting comprises: i) first contacting said cell with said first, second, third, fourth reagents and fifth reagents that comprise nucleic acids, and ii) subsequently treating said cell with said second reagent comprising a small molecule.

34. The method of claim 32, wherein said contacting comprises: i) electroporating said cell in a solution comprising said first, second, third, and fourth reagents, and ii) subsequently treating said cell with a vector encoding said fifth reagent.

35. The method of any of claim 32, further comprising glycerol as a sixth reagent, wherein said contacting said cell includes contacting with said sixth reagent.

36. The method of any of claim 32, wherein said region of interest replaces said disease/condition-causing mutation region during homology-directed repair (HDR) after said Cas nuclease cleaves said genomic DNA at said target site.

37-65. (canceled)

66. A composition comprising:
a) mRNA encoding a first protein, wherein said first protein is capable of reducing, suppressing or preventing an innate immune response in a human or animal cell, and
wherein a mutation in the genome of said cell is responsible for a disease or condition in a human or animal subject;
b) a second mRNA encoding a second protein that inhibits 53BPI,
c) mRNA encoding a third protein, wherein said third protein comprises a Cas nuclease for CRISPR; and
d) guide RNA (gRNA or sgRNA) comprising a tracrRNA having a sequence that binds to a Cas enzyme and a CRISPR RNA (crRNA) having a short “guide” sequence that is complementary to and binds to a specific target cleavage site in the genome that is near to said mutation.

67-95. (canceled)
